SEQUENCE LISTING (144 sequences)

SEQ ID NO: 1 - protein, 277 aa, synthetic construct
MKREREMLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAVGSMDSNYQLIWGAGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 2 - protein, 24 aa, synthetic construct
MKREREMLLITSMLVLWMQLSQVN

SEQ ID NO: 3 - protein, 112 aa, synthetic construct
GQQVMQIPQYQHVQEGEDFTTYCNSSTTLSNIQWYKQRPGGHPVFLIQLVKSGEVKKQKRLTFQFGEAKKNSSLHITATQTTDVGTYFCAVGSMDSNYQLIWGAGTKLIIKP

SEQ ID NO: 4 - protein, 141 aa, synthetic construct
DIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 5 - protein, 5 aa, synthetic construct
TTLSN

SEQ ID NO: 6 - protein, 7 aa, synthetic construct
LVKSGEV

SEQ ID NO: 7 - protein, 12 aa, synthetic construct
AVGSMDSNYQLI

SEQ ID NO: 8 - protein, 311 aa, synthetic construct
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSQERTWPYNEQFFGPGTRLTVLEDLKNVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 9 - protein, 19 aa, synthetic construct
MSLGLLCCGAFSLLWAGPV

SEQ ID NO: 10 - protein, 115 aa, synthetic construct
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSQERTWPYNEQFFGPGTRLTVL

SEQ ID NO: 11 - protein, 177 aa, synthetic construct
EDLKNVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 12 - protein, 5 aa, synthetic construct
MNHEY

SEQ ID NO: 13 - protein, 6 aa, synthetic construct
SVGEGT

SEQ ID NO: 14 - protein, 14 aa, synthetic construct
ASSQERTWPYNEQF

SEQ ID NO: 15 - protein, 5 aa, synthetic construct
TTTSN

SEQ ID NO: 16 - protein, 5 aa, synthetic construct
DASTE

SEQ ID NO: 17 - protein, 5 aa, synthetic construct
SRSGN

SEQ ID NO: 18 - protein, 7 aa, synthetic construct
WVKSGEV

SEQ ID NO: 19 - protein, 7 aa, synthetic construct
LPKSGEV

SEQ ID NO: 20 - protein, 7 aa, synthetic construct
LVLSGEV

SEQ ID NO: 21 - protein, 7 aa, synthetic construct
LVKSPEV

SEQ ID NO: 22 - protein, 7 aa, synthetic construct
LEKSGEV

SEQ ID NO: 23 - protein, 7 aa, synthetic construct
LGQSGEV

SEQ ID NO: 24 - protein, 7 aa, synthetic construct
  VARIANT 2: note = X can be any amino acid
  VARIANT 3: note = X can be any amino acid
LXXSGEV

SEQ ID NO: 25 - protein, 7 aa, synthetic construct
  VARIANT 2: note = X can be any amino acid
LXKSGEV

SEQ ID NO: 26 - protein, 14 aa, synthetic construct
CAVGSMDSNYQLIW

SEQ ID NO: 27 - protein, 12 aa, synthetic construct
AVGSWDSNYQLI

SEQ ID NO: 28 - protein, 14 aa, synthetic construct
CAVGSWDSNYQLIW

SEQ ID NO: 29 - protein, 12 aa, synthetic construct
AVGSRDSNYQLV

SEQ ID NO: 30 - protein, 14 aa, synthetic construct
CAVGSRDSNYQLVW

SEQ ID NO: 31 - protein, 12 aa, synthetic construct
AVGSRDSNYQLI

SEQ ID NO: 32 - protein, 14 aa, synthetic construct
CAVGSRDSNYQLIW

SEQ ID NO: 33 - protein, 12 aa, synthetic construct
AVGSMDSNYQLV

SEQ ID NO: 34 - protein, 14 aa, synthetic construct
CAVGSMDSNYQLVW

SEQ ID NO: 35 - protein, 12 aa, synthetic construct
AVGSNQRNYQLI

SEQ ID NO: 36 - protein, 14 aa, synthetic construct
CAVGSNQRNYQLIW

SEQ ID NO: 37 - protein, 12 aa, synthetic construct
AVGSNQDNYQLI

SEQ ID NO: 38 - protein, 14 aa, synthetic construct
CAVGSNQDNYQLIW

SEQ ID NO: 39 - protein, 12 aa, synthetic construct
AVGSNQENYQLI

SEQ ID NO: 40 - protein, 14 aa, synthetic construct
CAVGSNQENYQLIW

SEQ ID NO: 41 - protein, 12 aa, synthetic construct
AVGSNQMNYQLI

SEQ ID NO: 42 - protein, 14 aa, synthetic construct
CAVGSNQMNYQLIW

SEQ ID NO: 43 - protein, 12 aa, synthetic construct
AVGSNQVNYQLI

SEQ ID NO: 44 - protein, 14 aa, synthetic construct
CAVGSNQVNYQLIW

SEQ ID NO: 45 - protein, 12 aa, synthetic construct
AVGSRQSNYQLI

SEQ ID NO: 46 - protein, 14 aa, synthetic construct
CAVGSRQSNYQLIW

SEQ ID NO: 47 - protein, 12 aa, synthetic construct
AVGSMQSNYQLI

SEQ ID NO: 48 - protein, 14 aa, synthetic construct
CAVGSMQSNYQLIW

SEQ ID NO: 49 - protein, 12 aa, synthetic construct
  VARIANT 5: note = X can be any amino acid
  VARIANT 6: note = X can be any amino acid
  VARIANT 7: note = X can be any amino acid
  VARIANT 12: note = X can be any amino acid
AVGSXXXNYQLX

SEQ ID NO: 50 - protein, 12 aa, synthetic construct
  VARIANT 7: note = X can be any amino acid
AVGSNQXNYQLI

SEQ ID NO: 51 - protein, 14 aa, synthetic construct
CAVGSNQSNYQLIW

SEQ ID NO: 52 - protein, 6 aa, synthetic construct
  VARIANT 4: note = X can be any amino acid
SNQXNY

SEQ ID NO: 53 - skipped

SEQ ID NO: 54 - protein, 6 aa, synthetic construct
SMDSNY

SEQ ID NO: 55 - protein, 12 aa, synthetic construct
  VARIANT 5: note = X is M, N, R, or W
  VARIANT 6: note = X is D or Q
  VARIANT 7: note = X is D, E, M, R, S, or V
  VARIANT 12: note = X is I or V
AVGSXXXNYQLX

SEQ ID NO: 56 - protein, 6 aa, synthetic construct
SVGEGN

SEQ ID NO: 57 - protein, 16 aa, synthetic construct
CASSQERTWPYNEQFF

SEQ ID NO: 58 - protein, 14 aa, synthetic construct
ASSQERTQPYNEQF

SEQ ID NO: 59 - protein, 16 aa, synthetic construct
CASSQERTQPYNEQFF

SEQ ID NO: 60 - protein, 14 aa, synthetic construct
ASSQERTAPYNEQF

SEQ ID NO: 61 - protein, 16 aa, synthetic construct
CASSQERTAPYNEQFF

SEQ ID NO: 62 - protein, 14 aa, synthetic construct
  VARIANT 8: note = X can be any amino acid
ASSQERTXPYNEQF

SEQ ID NO: 63 - protein, 14 aa, synthetic construct
  VARIANT 8: note = X is any amino acid apart from W
ASSQERTXPYNEQF

SEQ ID NO: 64 - protein, 6 aa, synthetic construct
  VARIANT 4: note = X is A, Q, or W, or a conservative substitution thereof
ERTXPY

SEQ ID NO: 65 - protein, 6 aa, synthetic construct
ERTWPY

SEQ ID NO: 66 - protein, 14 aa, synthetic construct
  VARIANT 8: note = X is A, Q, or W
ASSQERTXPYNEQF

SEQ ID NO: 67 - protein, 85 aa, synthetic construct
IQNPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 68 - protein, 129 aa, synthetic construct
DLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 69 - protein, 15 aa, synthetic construct
GLNDIFEAQKIEWHE

SEQ ID NO: 70 - protein, 292 aa, synthetic construct
MGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPGLNDIFEAQKIEWHE

SEQ ID NO: 71 - protein, 26 aa, synthetic construct
GQQVMQIPQYQHVQEGEDFTTYCNSS

SEQ ID NO: 72 - protein, 17 aa, synthetic construct
IQWYKQRPGGHPVFLIQ

SEQ ID NO: 73 - protein, 33 aa, synthetic construct
KKQKRLTFQFGEAKKNSSLHITATQTTDVGTYF

SEQ ID NO: 74 - protein, 10 aa, synthetic construct
  VARIANT 8: note = X is I or V
GAGTKLIXKP

SEQ ID NO: 75 - protein, 26 aa, synthetic construct
NAGVTQTPKFRVLKTGQSMTLLCAQD

SEQ ID NO: 76 - protein, 17 aa, synthetic construct
  VARIANT 1: note = X is M or L
  VARIANT 9: note = X is D or G
XYWYRQDPXMGLRLIHY

SEQ ID NO: 77 - protein, 17 aa, synthetic construct
MYWYRQDPDMGLRLIHY

SEQ ID NO: 78 - protein, 17 aa, synthetic construct
MYWYRQDPGMGLRLIHY

SEQ ID NO: 79 - protein, 17 aa, synthetic construct
LYWYRQDPGMGLRLIHY

SEQ ID NO: 80 - protein, 36 aa, synthetic construct
  VARIANT 2: note = X is A or T
TXKGEVPDGYNVSRLKKQNFLLGLESAAPSQTSVYF

SEQ ID NO: 81 - protein, 9 aa, synthetic construct
GPGTRLTVL

SEQ ID NO: 82 - protein, 112 aa, synthetic construct
GQQVMQIPQYQHVQEGEDFTTYCNSSTTLSNIQWYKQRPGGHPVFLIQWVKSGEVKKQKRLTFQFGEAKKNSSLHITATQTTDVGTYFCAVGSMDSNYQLIWGAGTKLIIKP

SEQ ID NO: 83 - protein, 112 aa, synthetic construct
GQQVMQIPQYQHVQEGEDFTTYCNSSTTLSNIQWYKQRPGGHPVFLIQLPKSGEVKKQKRLTFQFGEAKKNSSLHITATQTTDVGTYFCAVGSMDSNYQLIWGAGTKLIIKP

SEQ ID NO: 84 - protein, 112 aa, synthetic construct
GQQVMQIPQYQHVQEGEDFTTYCNSSTTLSNIQWYKQRPGGHPVFLIQLVLSGEVKKQKRLTFQFGEAKKNSSLHITATQTTDVGTYFCAVGSMDSNYQLIWGAGTKLIIKP

SEQ ID NO: 85 - protein, 112 aa, synthetic construct
GQQVMQIPQYQHVQEGEDFTTYCNSSTTLSNIQWYKQRPGGHPVFLIQLVKSPEVKKQKRLTFQFGEAKKNSSLHITATQTTDVGTYFCAVGSMDSNYQLIWGAGTKLIIKP

SEQ ID NO: 86 - protein, 112 aa, synthetic construct
GQQVMQIPQYQHVQEGEDFTTYCNSSTTLSNIQWYKQRPGGHPVFLIQLVKSGEVKKQKRLTFQFGEAKKNSSLHITATQTTDVGTYFCAVGSWDSNYQLIWGAGTKLIIKP

SEQ ID NO: 87 - protein, 112 aa, synthetic construct
GQQVMQIPQYQHVQEGEDFTTYCNSSTTTSNIQWYKQRPGGHPVFLIQLVKSGEVKKQKRLTFQFGEAKKNSSLHITATQTTDVGTYFCAVGSRDSNYQLVWGAGTKLIIKP

SEQ ID NO: 88 - protein, 112 aa, synthetic construct
GQQVMQIPQYQHVQEGEDFTTYCNSSTTLSNIQWYKQRPGGHPVFLIQLVKSGEVKKQKRLTFQFGEAKKNSSLHITATQTTDVGTYFCAVGSRDSNYQLIWGAGTKLIVKP

SEQ ID NO: 89 - protein, 112 aa, synthetic construct
GQQVMQIPQYQHVQEGEDFTTYCNSSTTTSNIQWYKQRPGGHPVFLIQLVKSGEVKKQKRLTFQFGEAKKNSSLHITATQTTDVGTYFCAVGSMDSNYQLVWGAGTKLIVKP

SEQ ID NO: 90 - protein, 112 aa, synthetic construct
GQQVMQIPQYQHVQEGEDFTTYCNSSTTLSNIQWYKQRPGGHPVFLIQLVKSGEVKKQKRLTFQFGEAKKNSSLHITATQTTDVGTYFCAVGSRDSNYQLVWGAGTKLIVKP

SEQ ID NO: 91 - protein, 112 aa, synthetic construct
GQQVMQIPQYQHVQEGEDFTTYCNSSTTTSNIQWYKQRPGGHPVFLIQLVKSGEVKKQKRLTFQFGEAKKNSSLHITATQTTDVGTYFCAVGSRDSNYQLIWGAGTKLIVKP

SEQ ID NO: 92 - protein, 112 aa, synthetic construct
GQQVMQIPQYQHVQEGEDFTTYCNSSTTTSNIQWYKQRPGGHPVFLIQLVKSGEVKKQKRLTFQFGEAKKNSSLHITATQTTDVGTYFCAVGSRDSNYQLVWGAGTKLIVKP

SEQ ID NO: 93 - protein, 112 aa, synthetic construct
GQQVMQIPQYQHVQEGEDFTTYCNSSTTLSNIQWYKQRPGGHPVFLIQLVKSGEVKKQKRLTFQFGEAKKNSSLHITATQTTDVGTYFCAVGSRDSNYQLIWGAGTKLIIKP

SEQ ID NO: 94 - protein, 112 aa, synthetic construct
GQQVMQIPQYQHVQEGEDFTTYCNSSTTLSNIQWYKQRPGGHPVFLIQLVKSGEVKKQKRLTFQFGEAKKNSSLHITATQTTDVGTYFCAVGSNQRNYQLIWGAGTKLIIKP

SEQ ID NO: 95 - protein, 112 aa, synthetic construct
GQQVMQIPQYQHVQEGEDFTTYCNSSTTLSNIQWYKQRPGGHPVFLIQLVKSGEVKKQKRLTFQFGEAKKNSSLHITATQTTDVGTYFCAVGSNQDNYQLIWGAGTKLIIKP

SEQ ID NO: 96 - protein, 112 aa, synthetic construct
GQQVMQIPQYQHVQEGEDFTTYCNSSTTLSNIQWYKQRPGGHPVFLIQLVKSGEVKKQKRLTFQFGEAKKNSSLHITATQTTDVGTYFCAVGSNQENYQLIWGAGTKLIIKP

SEQ ID NO: 97 - protein, 112 aa, synthetic construct
GQQVMQIPQYQHVQEGEDFTTYCNSSTTLSNIQWYKQRPGGHPVFLIQLVKSGEVKKQKRLTFQFGEAKKNSSLHITATQTTDVGTYFCAVGSNQMNYQLIWGAGTKLIIKP

SEQ ID NO: 98 - protein, 112 aa, synthetic construct
GQQVMQIPQYQHVQEGEDFTTYCNSSTTLSNIQWYKQRPGGHPVFLIQLVKSGEVKKQKRLTFQFGEAKKNSSLHITATQTTDVGTYFCAVGSNQVNYQLIWGAGTKLIIKP

SEQ ID NO: 99 - protein, 112 aa, synthetic construct
GQQVMQIPQYQHVQEGEDFTTYCNSSTTTSNIQWYKQRPGGHPVFLIQLEKSGEVKKQKRLTFQFGEAKKNSSLHITATQTTDVGTYFCAVGSRDSNYQLVWGAGTKLIIKP

SEQ ID NO: 100 - protein, 112 aa, synthetic construct
GQQVMQIPQYQHVQEGEDFTTYCNSSTTTSNIQWYKQRPGGHPVFLIQLGQSGEVKKQKRLTFQFGEAKKNSSLHITATQTTDVGTYFCAVGSRDSNYQLVWGAGTKLIIKP

SEQ ID NO: 101 - protein, 112 aa, synthetic construct
GQQVMQIPQYQHVQEGEDFTTYCNSSTTTSNIQWYKQRPGGHPVFLIQLEKSGEVKKQKRLTFQFGEAKKNSSLHITATQTTDVGTYFCAVGSRDSNYQLIWGAGTKLIIKP

SEQ ID NO: 102 - protein, 112 aa, synthetic construct
GQQVMQIPQYQHVQEGEDFTTYCNSSTTTSNIQWYKQRPGGHPVFLIQLGQSGEVKKQKRLTFQFGEAKKNSSLHITATQTTDVGTYFCAVGSRDSNYQLIWGAGTKLIIKP

SEQ ID NO: 103 - protein, 112 aa, synthetic construct
GQQVMQIPQYQHVQEGEDFTTYCNSSTTTSNIQWYKQRPGGHPVFLIQLVKSGEVKKQKRLTFQFGEAKKNSSLHITATQTTDVGTYFCAVGSRDSNYQLIWGAGTKLIIKP

SEQ ID NO: 104 - protein, 112 aa, synthetic construct
GQQVMQIPQYQHVQEGEDFTTYCNSSTTTSNIQWYKQRPGGHPVFLIQLVKSGEVKKQKRLTFQFGEAKKNSSLHITATQTTDVGTYFCAVGSMDSNYQLIWGAGTKLIIKP

SEQ ID NO: 105 - protein, 112 aa, synthetic construct
GQQVMQIPQYQHVQEGEDFTTYCNSSDASTEIQWYKQRPGGHPVFLIQLEKSGEVKKQKRLTFQFGEAKKNSSLHITATQTTDVGTYFCAVGSRDSNYQLIWGAGTKLIIKP

SEQ ID NO: 106 - protein, 112 aa, synthetic construct
GQQVMQIPQYQHVQEGEDFTTYCNSSSRSGNIQWYKQRPGGHPVFLIQLGQSGEVKKQKRLTFQFGEAKKNSSLHITATQTTDVGTYFCAVGSRDSNYQLIWGAGTKLIIKP

SEQ ID NO: 107 - protein, 112 aa, synthetic construct
GQQVMQIPQYQHVQEGEDFTTYCNSSSRSGNIQWYKQRPGGHPVFLIQLVKSGEVKKQKRLTFQFGEAKKNSSLHITATQTTDVGTYFCAVGSRDSNYQLIWGAGTKLIIKP

SEQ ID NO: 108 - protein, 112 aa, synthetic construct
GQQVMQIPQYQHVQEGEDFTTYCNSSTTLSNIQWYKQRPGGHPVFLIQLGQSGEVKKQKRLTFQFGEAKKNSSLHITATQTTDVGTYFCAVGSRDSNYQLIWGAGTKLIIKP

SEQ ID NO: 109 - protein, 112 aa, synthetic construct
GQQVMQIPQYQHVQEGEDFTTYCNSSTTLSNIQWYKQRPGGHPVFLIQLEKSGEVKKQKRLTFQFGEAKKNSSLHITATQTTDVGTYFCAVGSRDSNYQLIWGAGTKLIIKP

SEQ ID NO: 110 - protein, 112 aa, synthetic construct
GQQVMQIPQYQHVQEGEDFTTYCNSSTTLSNIQWYKQRPGGHPVFLIQLVKSGEVKKQKRLTFQFGEAKKNSSLHITATQTTDVGTYFCAVGSRDSNYQLVWGAGTKLIIKP

SEQ ID NO: 111 - protein, 112 aa, synthetic construct
GQQVMQIPQYQHVQEGEDFTTYCNSSTTLSNIQWYKQRPGGHPVFLIQLGQSGEVKKQKRLTFQFGEAKKNSSLHITATQTTDVGTYFCAVGSRDSNYQLVWGAGTKLIIKP

SEQ ID NO: 112 - protein, 112 aa, synthetic construct
GQQVMQIPQYQHVQEGEDFTTYCNSSTTLSNIQWYKQRPGGHPVFLIQLEKSGEVKKQKRLTFQFGEAKKNSSLHITATQTTDVGTYFCAVGSRDSNYQLVWGAGTKLIIKP

SEQ ID NO: 113 - protein, 112 aa, synthetic construct
GQQVMQIPQYQHVQEGEDFTTYCNSSTTLSNIQWYKQRPGGHPVFLIQLVKSGEVKKQKRLTFQFGEAKKNSSLHITATQTTDVGTYFCAVGSRQSNYQLIWGAGTKLIIKP

SEQ ID NO: 114 - protein, 112 aa, synthetic construct
GQQVMQIPQYQHVQEGEDFTTYCNSSTTLSNIQWYKQRPGGHPVFLIQLPKSGEVKKQKRLTFQFGEAKKNSSLHITATQTTDVGTYFCAVGSMQSNYQLIWGAGTKLIIKP

SEQ ID NO: 115 - protein, 112 aa, synthetic construct
GQQVMQIPQYQHVQEGEDFTTYCNSSTTLSNIQWYKQRPGGHPVFLIQLPKSGEVKKQKRLTFQFGEAKKNSSLHITATQTTDVGTYFCAVGSRQSNYQLIWGAGTKLIIKP

SEQ ID NO: 116 - protein, 112 aa, synthetic construct
GQQVMQIPQYQHVQEGEDFTTYCNSSTTLSNIQWYKQRPGGHPVFLIQLPKSGEVKKQKRLTFQFGEAKKNSSLHITATQTTDVGTYFCAVGSRDSNYQLIWGAGTKLIIKP

SEQ ID NO: 117 - protein, 115 aa, synthetic construct
NAGVTQTPKFRVLKTGQSMTLLCAQDMNHEYLYWYRQDPGMGLRLIHYSVGEGTTAKGEVPDGYNVSRLKKQNFLLGLESAAPSQTSVYFCASSQERTWPYNEQFFGPGTRLTVL

SEQ ID NO: 118 - protein, 115 aa, synthetic construct
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSQERTQPYNEQFFGPGTRLTVL

SEQ ID NO: 119 - protein, 115 aa, synthetic construct
NAGVTQTPKFRVLKTGQSMTLLCAQDMNHEYMYWYRQDPDMGLRLIHYSVGEGTTTKGEVPDGYNVSRLKKQNFLLGLESAAPSQTSVYFCASSQERTWPYNEQFFGPGTRLTVL

SEQ ID NO: 120 - protein, 115 aa, synthetic construct
NAGVTQTPKFRVLKTGQSMTLLCAQDMNHEYMYWYRQDPGMGLRLIHYSVGEGNTAKGEVPDGYNVSRLKKQNFLLGLESAAPSQTSVYFCASSQERTWPYNEQFFGPGTRLTVL

SEQ ID NO: 121 - protein, 115 aa, synthetic construct
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSQERTAPYNEQFFGPGTRLTVL

SEQ ID NO: 122 - protein, 9 aa, synthetic construct
KVLEHVVRV

SEQ ID NO: 123 - protein, 100 aa, synthetic construct [sequence omitted]

SEQ ID NO: 124 - protein, 9 aa, Homo sapiens
FLLETVVRV

SEQ ID NO: 125 - protein, 9 aa, Homo sapiens
ILDEHVQRV

SEQ ID NO: 126 - protein, 9 aa, Homo sapiens
KILEDVVGV

SEQ ID NO: 127 - protein, 9 aa, Homo sapiens
KLLDLQVRV

SEQ ID NO: 128 - protein, 9 aa, Homo sapiens
KVLEILHRV

SEQ ID NO: 129 - protein, 9 aa, Homo sapiens
KVLERVNAV

SEQ ID NO: 130 - protein, 9 aa, Homo sapiens
KVLETLVTV

SEQ ID NO: 131 - protein, 9 aa, Homo sapiens
KVLGIVVGV

SEQ ID NO: 132 - protein, 9 aa, Homo sapiens
KVLNKVITV

SEQ ID NO: 133 - protein, 9 aa, Homo sapiens
KVTEMLVNV

SEQ ID NO: 134 - protein, 9 aa, Homo sapiens
KVLETTVEI

SEQ ID NO: 135 - protein, 9 aa, Homo sapiens
KVLEMETTV

SEQ ID NO: 136 - protein, 9 aa, Homo sapiens
KVMEHVYEK

SEQ ID NO: 137 - protein, 9 aa, Homo sapiens
KVQEQVHKV

SEQ ID NO: 138 - protein, 177 aa, synthetic construct
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 139 - protein, 179 aa, synthetic construct
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 140 - protein, 12 aa, synthetic construct
AVGSVNTNYQLI

SEQ ID NO: 141 - protein, 14 aa, synthetic construct
CAVGSVNTNYQLIW

SEQ ID NO: 142 - protein, 112 aa, synthetic construct
GQQVMQIPQYQHVQEGEDFTTYCNSSTTLSNIQWYKQRPGGHPVFLIQLVKSGEVKKQKRLTFQFGEAKKNSSLHITATQTTDVGTYFCAVGSVNTNYQLIWGAGTKLIIKP

SEQ ID NO: 143 - protein, 112 aa, synthetic construct
GQQVMQIPQYQHVQEGEDFTTYCNSSTTLSNIQWYKQRPGGHPVFLIQLEKSGEVKKQKRLTFQFGEAKKNSSLHITATQTTDVGTYFCAVGSVNTNYQLIWGAGTKLIIKP

SEQ ID NO: 144 - protein, 115 aa, synthetic construct
NAGVTQTPKFRVLKTGQSMTLLCAQDMNHEYMYWYRQDPDMGLRLIHYSVGEGTTTKGEVPDGYNVSRLKKQNFLLGLESAAPSQTSVYFCASSQERTQPYNEQFFGPGTRLTVL

(19) United States
(12) Patent Application Publication
(10) Pub. No.: US 2026/0226134 A1
(43) Pub. Date: Aug. 6, 2026

(54) ANTI-MAGE-A4 T CELL RECEPTORS

(71) Applicant: ZELLUNA IMMUNOTHERAPY AS, Oslo (NO)

(72) Inventors: Ross ROBINSON, Oslo (NO); Zahra OUARAY, Oslo (NO); Thomas DIXON, Oslo (NO); Margherita BOIERI, Oslo (NO); Syivie POLLMANN, Oslo (NO); Luise WEIGAND, Oslo (NO); Inês CARDOSE, Oslo (NO); Jacob HURST, Oslo (NO); Michelle TENG, Oslo (NO); Terje FRIGSTAD, Oslo (NO); Sebastian BERGE-SEIDL, Oslo (NO); Goril BERNTZEN, Oslo (NO); Geir Age LØSET, Oslo (NO)

(21) Appl. No.: 19/153,255

(22) PCT Filed: Feb. 1, 2024
(86) PCT No.: PCT/EP2024/052478

§ 371 Date: Aug. 1, 2025

(30) Foreign Application Priority Data
Feb. 3, 2023 (EP) 23154990.8

(51) Int. Cl. C07K 14/705; A61K 35/17; A61K 38/00; A61K 40/11; A61K 40/32; A61K 40/42; C12N 5/0783; C12N 15/63

(52) U.S. Cl. CPC C07K 14/7051; A61K 35/17; A61K 40/11; A61K 40/32; A61K 40/4267; C12N 5/0636; C12N 15/63; A61K 38/00; C07K 2317/565; C07K 2319/30; C12N 2510/00

(57) ABSTRACT

The present invention relates to T cell receptors (TCRs) and, in particular, TCRs specific for peptides derived from Melanoma-associated antigen 4 (MAGE-A4). The present invention also relates to antigen-binding portions of said TCRs, compositions comprising said TCRs or portions, fusion proteins comprising said TCRs or portions, nucleic acids and vectors encoding said TCRs or portions, cells comprising said TCRs or portions, and methods of treatment involving said TCRs or portions.

### FIELD OF THE INVENTION

The present invention relates to T cell receptors (TCRs) and, in particular, TCRs specific for peptides derived from Melanoma-associated antigen 4 (MAGE-A4). The present invention also relates to antigen-binding portions of said TCRs, compositions comprising said TCRs or portions, fusion proteins comprising said TCRs or portions, nucleic acids and vectors encoding said TCRs or portions, cells comprising said TCRs or portions, and methods of treatment involving said TCRs or portions.

### BACKGROUND OF THE INVENTION

MAGE-A4 is a cancer testis antigen. Cancer testis antigens are expressed in testis tissues and are also expressed by some types of cancer cell. This enables MAGE-A4 expression to be used as a marker of some cancerous cells.

MAGE-A4 expression by a particular cell may be recognised by a T cell expressing a TCR that is specific for a peptide derived from MAGE-A4 when presented in the context of an MHC molecule. TCRs with this property have been reported, such as in WO2021/236548 A1.

Other documents disclosing MAGE-A antigen-binding proteins include WO2021/023658A1 and WO2020/257288A2.

Such TCRs are useful for a range of purposes, including several types of immunotherapy. TCRs with a range of properties are required for the development of such therapies. Thus, there is a need for further MAGE-A4 specific TCRs.

### SUMMARY OF THE INVENTION

In an aspect, there is provided a T cell receptor (TCR), or an antigen-binding portion thereof, capable of binding specifically to a peptide according to SEQ ID NO: 122 presented in the context of an MHC class I molecule, comprising: a TCR-alpha (TCR-a) variable region, or portion thereof, and a TCR-beta (TCR-b) variable region, or portion thereof, wherein the TCR-a variable region, or portion thereof, comprises a complementarity determining region 3 alpha (CDR3a) comprising SEQ ID NO: 49 or SEQ ID NO: 53, wherein the CDR3a does not comprise SEQ ID NO: 7; and/or the TCR-b variable region, or portion thereof, comprises a complementarity determining region 3 beta (CDR3b) comprising SEQ ID NO: 62 or SEQ ID NO: 64, wherein the CDR3b does not comprise SEQ ID NO: 14.

In an embodiment, the CDR3a comprises SEQ ID NO: 49 or SEQ ID NO: 53 and does not comprise SEQ ID NO: 7, and the TCR-b variable region, or portion thereof, comprises a CDR3b comprising any one of SEQ ID NOs: 14 and 57 to 66.

In another embodiment, the TCR-a variable region, or portion thereof, comprises a CDR3a comprising any one of SEQ ID NOs: 7, 26 to 55, 140, and 141 or optionally any one of SEQ ID NOs: 7 and 26 to 55, and the CDR3b comprises SEQ ID NO: 62 or SEQ ID NO: 64, wherein the CDR3b does not comprise SEQ ID NO: 14.

The TCR-a variable region, or portion thereof, may comprise a CDR1a comprising any one of SEQ ID NOs: 5 and 15 to 17. The TCR-a variable region, or portion thereof, may comprise a CDR2a comprising any one of SEQ ID NOs: 6 and 18 to 25. The TCR-b variable region, or portion thereof, may comprise a CDR1b comprising SEQ ID NO: 12. The TCR-b variable region, or portion thereof, may comprise a CDR2b comprising SEQ ID NO: 13 or SEQ ID NO: 56.

In another aspect, there is provided a TCR, or an antigen-binding portion thereof, capable of binding specifically to a peptide according to SEQ ID NO: 122 presented in the context of an MHC class I molecule, comprising a TCR-a variable region, or portion thereof, and a TCR-b variable region, or portion thereof, wherein the TCR-a variable region, or portion thereof, comprises a CDR3a and the TCR-b variable region, or portion thereof, comprises a CDR3b; the CDR3a comprises any one of the CDR3a sequences recited in Table 1; the CDR3b comprises any one of the CDR3b sequences recited in Table 2; and the CDR3a and CDR3b are associated with the same TCR ID listed in Tables 1 and 2.

In another aspect, there is provided a TCR, or an antigen-binding portion thereof, capable of binding specifically to a peptide according to SEQ ID NO: 122 presented in the context of an MHC class I molecule, comprising: a TCR-a variable region, or portion thereof, and a TCR-b variable region, or portion thereof, wherein the TCR-a variable region, or portion thereof, comprises a CDR1a, a CDR2a, and a CDR3a and the TCR-b variable region, or portion thereof, comprises a CDR1b, a CDR2b, and a CDR3b; and the CDR1a comprises any one of the CDR1a sequences recited in Table 1; the CDR2a comprises any one of the CDR2a sequences recited in Table 1; the CDR3a comprises any one of the CDR3a sequences recited in Table 1; the CDR1b comprises any one of the CDR1b sequences recited in Table 2; the CDR2b comprises any one of the CDR2b sequences recited in Table 2; the CDR3b comprises any one of the CDR3b sequences recited in Table 2; and the CDR1a, CDR2a, CDR3a, CDR1b, CDR2b, and CDR3b are all associated with the same TCR ID listed in Tables 1 and 2 (i.e. derived from the same clone).

The TCR-a variable region may comprise a sequence at least 70%, 80%, 90%, 95%, or 100% identical to SEQ ID NO: 71, a sequence at least 70%, 80%, 90%, 95%, or 100% identical to SEQ ID NO: 72, a sequence at least 70%, 80%, 90%, 95%, or 100% identical to SEQ ID NO: 73, and a sequence at least 70%, 80%, 90%, 95%, or 100% identical to SEQ ID NO: 74; and/or the TCR-b variable region may comprise a sequence at least 70%, 80%, 90%, 95%, or 100% identical to SEQ ID NO: 75, a sequence at least 70%, 80%, 90%, 95%, or 100% identical to any one of SEQ ID NOs: 76 to 79, a sequence at least 70%, 80%, 90%, 95%, or 100% identical to SEQ ID NO: 80, and a sequence at least 70%, 80%, 90%, 95%, or 100% identical to SEQ ID NO: 81.

The TCR-a variable region may comprise a sequence that is at least 70%, 80%, 85%, 90%, 95%, 97%, 98%, 99%, or 100% identical to any one of SEQ ID NOs: 3, 82 to 116, 142, and 143, or a portion thereof; and/or the TCR-b variable region may comprise a sequence that is at least 70%, 80%, 85%, 90%, 95%, 97%, 98%, 99%, or 100% identical to any one of SEQ ID NOs: 10, 117 to 121, and 144, or a portion thereof. The TCR-a variable region may comprise a sequence that is at least 70%, 80%, 85%, 90%, 95%, 97%, 98%, 99%, or 100% identical to any one of SEQ ID NOs: 3 and 82 to 116, or a portion thereof; and/or the TCR-b variable region may comprise a sequence that is at least 70%, 80%, 85%, 90%, 95%, 97%, 98%, 99%, or 100% identical to any one of SEQ ID NOs: 10 and 117 to 121, or a portion thereof.

In another aspect, there is provided a TCR, or an antigen-binding portion thereof, capable of binding specifically to a peptide according to SEQ ID NO: 122 presented in the context of an MHC class I molecule, comprising: a TCR-a variable region that comprises a sequence that is at least 70%, 80%, 85%, 90%, 95%, 97%, 98%, 99%, or 100% identical to any one of SEQ ID NOs: 3, 82 to 116, 142, and 143, or a portion thereof; and a TCR-b variable region that comprises a sequence that is at least 70%, 80%, 85%, 90%, 95%, 97%, 98%, 99%, or 100% identical to any one of SEQ ID NOs: 10, 117 to 121, and 144, or a portion thereof; wherein the TCR does not comprise both SEQ ID NO: 3 and SEQ ID NO: 10 and/or wherein TCR-a variable region sequence and the TCR-b variable region sequence are associated with the same TCR ID listed in Tables 3 and 4. In examples, the CDR3a sequence is invariant and the CDR3b sequence is invariant, wherein the CDR3a sequence is defined in Table 3 and the CDR3b sequence is defined in Table 4; and/or the CDR1a, CDR2a, and CDR3a sequences are invariant and the CDR1b, CDR2b, and CDR3b sequences are invariant; wherein the CDR sequences are defined in Table 3 and Table 4; and/or the relevant mutations recited in Table 6 are retained.

In an aspect, there is provided a nucleic acid molecule encoding: (a) the TCRa variable region amino acid sequence; (b) the TCRb variable region amino acid sequence; or (c) both the TCRa variable region and the TCRb variable region amino acid sequences of any the TCR or antigen-binding portion disclosed herein.

In an aspect, there is provided vector comprising any nucleic acid or nucleic acids disclosed herein.

In an aspect, there is provided a cell comprising any TCR, or antigen-binding portion thereof, disclosed herein, any nucleic acid or nucleic acids disclosed herein, or any vector disclosed herein.

In an aspect, there is provided a pharmaceutical composition comprising any TCR, or antigen-binding portion thereof, disclosed herein, any nucleic acid or nucleic acids disclosed herein, any vector disclosed herein, or any cell disclosed herein.

In an aspect, there is provided any TCR, or antigen-binding portion thereof, disclosed herein, any nucleic acid or nucleic acids disclosed herein, any vector disclosed herein, any cell disclosed herein, or any pharmaceutical composition disclosed herein, for use as a medicament.

In an aspect, there is provided any TCR, or antigen-binding portion thereof, disclosed herein, any nucleic acid or nucleic acids disclosed herein, any vector disclosed herein, any cell disclosed herein, or any pharmaceutical composition disclosed herein, for use in a method for treating, preventing, or ameliorating cancer. In examples, the cancer comprises cells that express MAGE-A4. In examples, the cancer is non-small cell lung cancer (NSCLC), ovarian, gastroesophageal, head and neck synovial sarcoma, or urothelial.

### BRIEF DESCRIPTION OF THE DRAWINGS

FIG. 1 provides graphs showing the calculation of the K_{d} data in Example 6 for selected TCR clones.

FIG. 2 provides graphs showing the calculation of the EC₅₀ data in Example 6 for selected TCR clones.

FIG. 3 provides graphs showing the response to NCI-H1703 for selected TCR clones, as discussed in Example 6.

FIG. 4 provides a graph showing the response to NCI-H1703 for selected TCR clones, as discussed in Example 7.

### DETAILED DESCRIPTION

The inventors provide herein TCRs and, in particular, MAGE-A4 specific TCRs. The provided TCRs are capable of binding specifically to a peptide derived from the MAGE-A4 antigen when presented by an appropriate MHC molecule. Also provided are antigen-binding portions of said TCRs.

Thus, in an aspect, there is provided a TCR, or an antigen-binding portion thereof, capable of binding specifically to a peptide of the sequence KVLEHVVRV (SEQ ID NO: 122) presented in the context of an MHC class I molecule, comprising:
a TCR-alpha (TCR-a) variable region, or portion thereof, and a TCR-beta (TCR-b) variable region, or portion thereof, wherein
the TCR-a variable region, or portion thereof, comprises a complementarity determining region 3 alpha (CDR3a) comprising SEQ ID NO: 49 or SEQ ID NO: 53, wherein the CDR3a does not comprise SEQ ID NO: 7; and/or
the TCR-b variable region, or portion thereof, comprises a complementarity determining region 3 beta (CDR3b) comprising SEQ ID NO: 62 or SEQ ID NO: 64, wherein the CDR3b does not comprise SEQ ID NO: 14.

Hence, in an example, there is provided there is provided a TCR, or an antigen-binding portion thereof, capable of binding specifically to a peptide of the sequence KVLEHVVRV (SEQ ID NO: 122) presented in the context of an MHC class I molecule, comprising:
a TCR-a variable region, or portion thereof, and a TCR-b variable region, or portion thereof, wherein
the TCR-a variable region, or portion thereof, comprises a CDR3a comprising SEQ ID NO: 49 or SEQ ID NO: 53, wherein the CDR3a does not comprise SEQ ID NO: 7.

In an embodiment, the TCR-a variable region, or portion thereof, comprises a CDR3a comprising or according to SEQ ID NO: 49, wherein the CDR3a does not comprise SEQ ID NO: 7, and the TCR-b variable region comprises any CDR3b disclosed herein. For instance, a CDR3b comprising or according to any one of SEQ ID NOs: 14 and 57 to 66.

In another embodiment, the TCR-a variable region, or portion thereof, comprises a CDR3a comprising or according to SEQ ID NO: 53, wherein the CDR3a does not comprise SEQ ID NO: 54, and the TCR-b variable region comprises any CDR3b disclosed herein. For instance, a CDR3b comprising or according to any one of SEQ ID NOs: 14 and 57 to 66.

Additionally, there is provided a TCR, or an antigen-binding portion thereof, capable of binding specifically to a peptide of the sequence KVLEHVVRV (SEQ ID NO: 122) presented in the context of an MHC class I molecule, comprising:
a TCR-a variable region, or portion thereof, and a TCR-b variable region, or portion thereof, wherein
the TCR-b variable region, or portion thereof, comprises a CDR3b comprising SEQ ID NO: 62 or SEQ ID NO: 64, wherein the CDR3b does not comprise SEQ ID NO: 14.

In an embodiment, the TCR-a variable region, or portion thereof, comprises any CDR3a disclosed herein, and the TCR-b variable region, or portion thereof, comprises a CDR3b comprising SEQ ID NO: 62, wherein the CDR3b does not comprise SEQ ID NO: 14. In some examples, the CDR3a comprises any one of SEQ ID NOs: 7, 26 to 55, 140, and 141 or optionally any one of SEQ ID NOs: 7 and 26 to 55.

In an embodiment, the TCR-a variable region, or portion thereof, comprises any CDR3a disclosed herein, and the TCR-b variable region, or portion thereof, comprises a CDR3b comprising SEQ ID NO: 64, wherein the CDR3b does not comprise SEQ ID NO: 65. In some examples, the CDR3a comprises any one of SEQ ID NOs: 7, 26 to 55, 140, and 141 or optionally any one of SEQ ID NOs: 7 and 26 to 55.

The TCR, or antigen-binding portion thereof, may comprise a CDR3a comprising or according to SEQ ID NO: 49, as follows: AVGSX₁X₂X₃NYQLX₄, wherein:
X₁ is any amino acid,
X₂ is any amino acid,
X₃ is any amino acid, and
X₄ is any amino acid. In some examples, the CDR3a does not comprise AVGSMDSNYQLI (SEQ ID NO: 7). In some examples, the amino acids of X₁-X₄ can be any naturally occurring or canonical amino acid. In examples, X₁ is M, N, R, V, or W, or a conservative substitution for any of said residues; X₂ is D, N, or Q, or a conservative substitution for any of said residues; X₃ is D, E, M, R, S, T, or V, or a conservative substitution for any of said residues; and/or X₄ is I or V, or a conservative substitution for any of said residues. In examples, X₁ is M, N, R, or W, or a conservative substitution for any of said residues; X₂ is D or Q, or a conservative substitution for any of said residues; X₃ is D, E, M, R, S, or V, or a conservative substitution for any of said residues; and/or X₄ is I or V, or a conservative substitution for any of said residues. In examples, X₁ is M, N, R, V, or W; X₂ is D, N, or Q; X₃ is D, E, M, R, S, T, or V; and X₄ is I or V. In examples, X₁ is M, N, R, or W; X₂ is D or Q; X₃ is D, E, M, R, S, or V; and X₄ is I or V. In other examples, X₁ is N, X₂ is Q, and X₃ and X₄ are any amino acid. In an example, X₁ is N, X₂ is Q, X₃ is any amino acid, and X₄ is I. In a particular example, X₁ is N, X₂ is Q, X₃ is D, E, M, R, S, or V, or a conservative substitution for any of said residues, and X₄ is I. In other examples, X₁ is V, X₂ is N, and X₃ is T; or X₁ is V, X₂ is N, X₃ is T, and X₄ is I.

The TCR, or antigen-binding portion thereof, may comprise a CDR3a comprising or according to SEQ ID NO: 50, as follows: AVGSNQX₁NYQLI, wherein: X₁ is any amino acid. In some examples, the amino acid of X₁ can be any naturally occurring or canonical amino acid. In examples, X₁ is D, E, M, R, S, or V, or a conservative substitution for any of said residues.

As discussed in the Examples, the fourth, fifth, sixth, eight, and ninth residues of CDR3a (numbered according to SEQ ID NO: 7-AVGSMDSNYQLI) are important for interacting with the peptide-HLA complex. Thus, the TCR, or antigen-binding portion thereof, may comprise a CDR3a comprising SEQ ID NO: 53, as follows:
SX₁X₂X₃NY, wherein:
X₁ is M, N, R, V, or W, or a conservative substitution thereof,
X₂ is D, N, or Q, or a conservative substitution thereof, and
X₃ is any amino acid. In some examples, X₁ is M, N, R, or W or a conservative substitution thereof and X₂ is D or Q or a conservative substitution thereof. In some examples, X₁ is M, N, R, V, or W and X₂ is D, N, or Q. In some examples, X₁ is M, N, R, or W and X₂ is D or Q. In some examples, the CDR3a does not comprise SMDSNY (SEQ ID NO: 54). In some examples, X₃ can be any naturally occurring or canonical amino acid. In examples, X₃ is D, E, M, R, S, T, or V, or a conservative substitution. In examples, X₃ is D, E, M, R, S, or V, or a conservative substitution. In a particular example, X₁ is M, N, R, V, or W; X₂ is D, N, or Q; and X₃ is D, E, M, R, S, T, or V. In another particular example, X₁ is M, N, R, or W; X₂ is D, Q, or S; and X₃ is D, E, M, R, S, or V. In other examples, X₁ is N, X₂ is Q, and X₃ is any amino acid. In a particular example, X₁ is N, X₂ is Q, and X₃ is D, E, M, R, S, or V, or a conservative substitution for any of said residues. In other examples, X₁ is V, X₂ is N, and X₃ is T. In some examples, the CDR3a includes one, two, three, four, or five additional residues at the N-terminal side and/or the C-terminal side. The additional residues may be those contiguous to the recited sequence within SEQ ID NO: 3, i.e. CDR3a may include a longer sequence from the parent clone.

The TCR, or antigen-binding portion thereof, may comprise a CDR3a comprising or according to SEQ ID NO: 52, as follows: SNQX₁NY, wherein: X₁ is any amino acid. In some examples, the amino acid of X₁ can be any naturally occurring or canonical amino acid. In examples, X₁ is D, E, M, R, S, or V, or a conservative substitution for any of said residues.

The TCR, or antigen-binding portion thereof, may comprise a CDR3a comprising or according to SEQ ID NO: 55, as follows: AVGSX₁X₂X₃NYQLX₄, wherein:
X₁ is M, N, R, or W,
X₂ is D or Q,
X₃ is D, E, M, R, S, or V, and
X₄ is I or V. In some embodiments, the CDR3a does not comprise AVGSMDSNYQLI (SEQ ID NO: 7).

The TCR, or antigen-binding portion thereof, may comprise any CDR3a sequence recited in Table 1. The TCR, or antigen-binding portion thereof, may comprise a CDR3a comprising or according to any one of SEQ ID NOs: 26 to 48, 140, and 141. The TCR, or antigen-binding portion thereof, may comprise a CDR3a comprising or according to any one of SEQ ID NOs: 7, 26 to 55, 140, and 141.

The TCR, or antigen-binding portion thereof, may comprise a CDR3b comprising or according to SEQ ID NO: 62, as follows: ASSQERTX₁PYNEQF, wherein:

X₁ is any amino acid. In some embodiments, the CDR3b does not comprise ASSQERTWPYNEQF (SEQ ID NO: 14). In some examples, the amino acid of X₁ can be any naturally occurring or canonical amino acid. In examples, X₁ is A, Q, or W, or a conservative substitution for any of said residues. In an example, X₁ is A, Q, or W.

The TCR, or antigen-binding portion thereof, may comprise a CDR3b comprising or according to SEQ ID NO: 63, as follows: ASSQERTX₁PYNEQF, wherein X₁ is any amino acid apart from W. In examples, X₁ is A or Q, or a conservative substitution for any of said residues. In an example, X₁ is A or Q.

As discussed in the Examples, the fifth to the tenth residues of CDR3b (numbered according to SEQ ID NO: 14—ASSQERTWPYNEQF) are important for interacting with the peptide-HLA complex. Thus, the TCR, or antigen-binding portion thereof, may comprise a CDR3b comprising SEQ ID NO: 64, as follows: ERTX₁PY, wherein X₁ is A, Q, or W, or a conservative substitution thereof. In an example, X₁ is A, Q, or W. In some embodiments, the CDR3b does not comprise ERTWPY (SEQ ID NO: 65). In some examples, the CDR3b includes one, two, three, four, five, six, or seven additional residues at the N-terminal side and/or the C-terminal side. The additional residues may be those contiguous to the recited sequence within SEQ ID NO: 10, i.e. CDR3b may include a longer sequence from the parent clone.

The TCR, or antigen-binding portion thereof, may comprise a CDR3b comprising or according to SEQ ID NO: 66, as follows: ASSQERTX₁PYNEQF, wherein X₁ is A, Q, or W. In some embodiments, the CDR3b does not comprise ASSQERTWPYNEQF (SEQ ID NO: 14).

The TCR, or antigen-binding portion thereof, may comprise any CDR3b sequence recited in Table 2. The TCR, or antigen-binding portion thereof, may comprise a CDR3b comprising or according to any one of SEQ ID NOs: 57 to 61. The TCR, or antigen-binding portion thereof, may comprise a CDR3b comprising or according to any one of SEQ ID NOs: 14 and 57 to 66.

The TCR, or antigen-binding portion thereof, may comprise any CDR1a sequence recited in Table 1. The TCR, or antigen-binding portion thereof, may comprise a CDR1a comprising or according to any one of SEQ ID NOs: 15 to 17. The TCR, or antigen-binding portion thereof, may comprise a CDR1a comprising or according to any one of SEQ ID NOs: 5 and 15 to 17.

The TCR, or antigen-binding portion thereof, may comprise a CDR2a. Specifically, the TCRa variable chain, or antigen-binding portion thereof, may comprise a CDR2a. The CDR2a may comprise or be according to SEQ ID NO: 24, as follows: LX₁X₂SGEV, wherein:
X₁ is any amino acid, and
X₂ is any amino acid. In some embodiments, CDR2a does not comprise LVKSGEV (SEQ ID NO: 6). In some examples, the amino acids of X₁-X₂ can be any naturally occurring or canonical amino acid. In examples, X₁ is E, G, P, or V, or a conservative substitution for any of said residues and/or X₂ is K, L, or Q, or a conservative substitution for any of said residues. In an example, X₁ is E, G, P, or V and X₂ is K, L, or Q.

The TCR, or antigen-binding portion thereof, may comprise a CDR2a. Specifically, the TCRa variable chain, or antigen-binding portion thereof, may comprise a CDR2a. The CDR2a may comprise or be according to SEQ ID NO: 25, as follows: LX₁KSGEV, wherein:

X₁ is any amino acid. In some embodiments, CDR2a does not comprise LVKSGEV (SEQ ID NO: 6). In some examples, the amino acid of X₁ can be any naturally occurring or canonical amino acid. In examples, X₁ is E, G, P, or V, or a conservative substitution for any of said residues. In an example, X₁ is E, G, P, or V.

The TCR, or antigen-binding portion thereof, may comprise any CDR2a sequence recited in Table 1. The TCR, or antigen-binding portion thereof, may comprise a CDR2a comprising or according to any one of SEQ ID NOs: 18 to 23. The TCR, or antigen-binding portion thereof, may comprise a CDR2a comprising or according to any one of SEQ ID NOs: 6 and 18 to 25.

The TCR, or antigen-binding portion thereof, may comprise the CDR1b sequence recited in Table 2. The TCR, or antigen-binding portion thereof, may comprise a CDR1b comprising or according SEQ ID NO: 12.

The TCR, or antigen-binding portion thereof, may comprise any CDR2b sequence recited in Table 2. The TCR, or antigen-binding portion thereof, may comprise a CDR2b comprising or according to SEQ ID NO: 56. The TCR, or antigen-binding portion thereof, may comprise a CDR2b comprising or according to SEQ ID NO: 13 or SEQ ID NO: 56.

In some embodiments, there is provided a TCR, or an antigen-binding portion thereof, comprising:
a TCR-a variable region, or an antigen-binding portion thereof, and a TCR-b variable region, or an antigen-binding portion thereof, wherein
the TCR-a variable region, or antigen-binding portion thereof, comprises a CDR3a comprising or according to SEQ ID NO: 49, and
the TCR-b variable region, or antigen-binding portion thereof, comprises a CDR3b comprising or according to SEQ ID NO: 62;

wherein the CDR3a does not comprise AVGSMDSNYQLI (SEQ ID NO: 7) and/or the CDR3b does not comprise ASSQERTWPYNEQF (SEQ ID NO: 14).

In some embodiments, there is provided a TCR, or an antigen-binding portion thereof, comprising:
a TCR-a variable region, or portion thereof, and a TCR-b variable region, or portion thereof, wherein
the TCR-a variable region, or portion thereof, comprises a CDR3a comprising or according to SEQ ID NO: 50, and
the TCR-b variable region, or portion thereof, comprises a CDR3b comprising or according to SEQ ID NO: 66.

In some embodiments, there is provided a TCR, or an antigen-binding portion thereof, comprising:
a TCR-a variable region, or portion thereof, and a TCR-b variable region, or portion thereof, wherein
the TCR-a variable region, or portion thereof, comprises a CDR3a comprising or according to SEQ ID NO: 53 (SX₁X₂X₃NY), wherein X₁ is V, X₂ is N, and X₃ is T, and
the TCR-b variable region, or portion thereof, comprises a CDR3b comprising or according to SEQ ID NO: 66.

In some embodiments, there is provided a TCR, or an antigen-binding portion thereof, comprising:
a TCR-a variable region, or a portion thereof, and a TCR-b variable region, or a portion thereof, wherein
the TCR-a variable region, or portion thereof, comprises a CDR3a comprising or according to any one of SEQ ID NOs: 7, 26 to 55, 140, and 141 or optionally any one of SEQ ID NOs: 7 and 26 to 55, and
the TCR-b variable region, or portion thereof, comprises a CDR1b comprising or according to SEQ ID NO: 12, a CDR2b comprising or according to SEQ ID NO: 13 or 56, and a CDR3b comprising or according to any one of SEQ ID NOs: 14 and 57 to 66;

wherein the CDR3a does not comprise SEQ ID NO: 7, the CDR2b does not comprise SEQ ID NO: 13, or the CDR3b does not comprise SEQ ID NO: 14.

In some embodiments, there is provided a TCR, or an antigen-binding portion thereof, comprising:
a TCR-a variable region, or a portion thereof, and a TCR-b variable region, or a portion thereof, wherein
the TCR-a variable region, or portion thereof, comprises a CDR1a comprising or according to any one of SEQ ID NOs: 5 and 15-17, a CDR2a comprising or according to any one of SEQ ID NOs: 6 and 18-25, and a CDR3a comprising or according to any one of SEQ ID NOs: 7, 26 to 55, 140, and 141 or optionally any one of SEQ ID NOs: 7 and 26 to 55, and
the TCR-b variable region, or portion thereof, comprises a CDR3b comprising or according to any one of SEQ ID NOs: 14 and 57 to 66;

wherein the CDR1a does not comprise SEQ ID NO: 5, the CDR2a does not comprise SEQ ID NO: 6, the CDR3a does not comprise SEQ ID NO: 7, or the CDR3b does not comprise SEQ ID NO: 14.

In some embodiments, there is provided a TCR, or an antigen-binding portion thereof, comprising:
a TCR-a variable region, or a portion thereof, and a TCR-b variable region, or a portion thereof, wherein
the TCR-a variable region, or portion thereof, comprises a CDR1a comprising or according to any one of SEQ ID NOs: 5 and 15-17, a CDR2a comprising or according to any one of SEQ ID NOs: 6 and 18-25, and a CDR3a comprising or according to any one of SEQ ID NOs: 7, 26 to 55, 140, and 141 or optionally any one of SEQ ID NOs: 7 and 26 to 55, and
the TCR-b variable region, or portion thereof, comprises a CDR1b comprising or according to SEQ ID NO: 12, a CDR2b comprising or according to SEQ ID NO: 13 or 56, and a CDR3b comprising or according to any one of SEQ ID NOs: 14 and 57 to 66;

wherein the CDR1a does not comprise SEQ ID NO: 5, the CDR2a does not comprise SEQ ID NO: 6, the CDR3a does not comprise SEQ ID NO: 7, the CDR2b does not comprise SEQ ID NO: 13, or the CDR3b does not comprise SEQ ID NO: 14.

In some embodiments, there is provided a TCR, or an antigen-binding portion thereof, comprising:
a TCR-a variable region, or a portion thereof, and a TCR-b variable region, or a portion thereof, wherein
the TCR-a variable region, or portion thereof, comprises a CDR1a comprising or according to any one of SEQ ID NOs: 5 and 15-17, a CDR2a comprising or according to any one of SEQ ID NOs: 6 and 18-23, and a CDR3a comprising or according to any one of SEQ ID NOs: 7, 26 to 48, 140, and 141 or optionally any one of SEQ ID NOs: 7 and 26 to 48, and
the TCR-b variable region, or portion thereof, comprises a CDR1b comprising or according to SEQ ID NO: 12, a CDR2b comprising or according to SEQ ID NO: 13 or 56, and a CDR3b comprising or according to any one of SEQ ID NOs: 14 and 57 to 61;

wherein the CDR1a does not comprise SEQ ID NO: 5, the CDR2a does not comprise SEQ ID NO: 6, the CDR3a does not comprise SEQ ID NO: 7, the CDR2b does not comprise SEQ ID NO: 13, or the CDR3b does not comprise SEQ ID NO: 14. In some embodiments, the CDRs are associated with the same TCR ID in Tables 1 and 2, i.e. are derived from the same clone.

TABLE 1

| | SEQ ID NO | | | | CDR3a- |
|---|---|---|---|---|---|
| | (in order left | | | | alternative |
| TCR ID | to right) | CDR1a | CDR2a | CDR3a | definition |
| KVL-01-018 | 5, 18, 7, 26 | TTLSN | WVKSGEV | AVGSMDSNYQLI | CAVGSMDSNYQLIW |
| KVL-01-019 | 5, 19, 7, 26 | TTLSN | LPKSGEV | AVGSMDSNYQLI | CAVGSMDSNYQLIW |
| KVL-01-020 | 5, 20, 7, 26 | TTLSN | LVLSGEV | AVGSMDSNYQLI | CAVGSMDSNYQLIW |
| KVL-01-022 | 5, 21, 7, 26 | TTLSN | LVKSPEV | AVGSMDSNYQLI | CAVGSMDSNYQLIW |
| KVL-01-026 | 5, 6, 27, 28 | TTLSN | LVKSGEV | AVGSWDSNYQLI | CAVGSWDSNYQLIW |
| KVL-01-034 | 15, 6, 29, 30 | TTTSN | LVKSGEV | AVGSRDSNYQLV | CAVGSRDSNYQLVW |
| KVL-01-036 | 5, 6, 31, 32 | TTLSN | LVKSGEV | AVGSRDSNYQLI | CAVGSRDSNYQLIW |
| KVL-01-039 | 15, 6, 33, 34 | TTTSN | LVKSGEV | AVGSMDSNYQLV | CAVGSMDSNYQLVW |
| KVL-01-040 | 5, 6, 29, 30 | TTLSN | LVKSGEV | AVGSRDSNYQLV | CAVGSRDSNYQLVW |
| KVL-01-041 | 15, 6, 31, 32 | TTTSN | LVKSGEV | AVGSRDSNYQLI | CAVGSRDSNYQLIW |
| KVL-01-042 | 15, 6, 29, 30 | TTTSN | LVKSGEV | AVGSRDSNYQLV | CAVGSRDSNYQLVW |
| KVL-01-043 | 5, 6, 31, 32 | TTLSN | LVKSGEV | AVGSRDSNYQLI | CAVGSRDSNYQLIW |
| KVL-01-048 | 5, 18, 7, 26 | TTLSN | WVKSGEV | AVGSMDSNYQLI | CAVGSMDSNYQLIW |
| KVL-01-049 | 5, 19, 7, 26 | TTLSN | LPKSGEV | AVGSMDSNYQLI | CAVGSMDSNYQLIW |
| KVL-01-050 | 5, 20, 7, 26 | TTLSN | LVLSGEV | AVGSMDSNYQLI | CAVGSMDSNYQLIW |
| KVL-01-052 | 5, 21, 7, 26 | TTLSN | LVKSPEV | AVGSMDSNYQLI | CAVGSMDSNYQLIW |
| KVL-01-056 | 5, 6, 27, 28 | TTLSN | LVKSGEV | AVGSWDSNYQLI | CAVGSWDSNYQLIW |
| KVL-01-060 | 5, 6, 7, 26 | TTLSN | LVKSGEV | AVGSMDSNYQLI | CAVGSMDSNYQLIW |
| KVL-01-061 | 5, 6, 31, 32 | TTLSN | LVKSGEV | AVGSRDSNYQLI | CAVGSRDSNYQLIW |
| KVL-01-068 | 5, 6, 35, 36 | TTLSN | LVKSGEV | AVGSNQRNYQLI | CAVGSNQRNYQLIW |
| KVL-01-069 | 5, 6, 37, 38 | TTLSN | LVKSGEV | AVGSNQDNYQLI | CAVGSNQDNYQLIW |
| KVL-01-070 | 5, 6, 39, 40 | TTLSN | LVKSGEV | AVGSNQENYQLI | CAVGSNQENYQLIW |
| KVL-01-071 | 5, 6, 41, 42 | TTLSN | LVKSGEV | AVGSNQMNYQLI | CAVGSNQMNYQLIW |
| KVL-01-073 | 5, 6, 35, 36 | TTLSN | LVKSGEV | AVGSNQRNYQLI | CAVGSNQRNYQLIW |
| KVL-01-074 | 5, 6, 37, 38 | TTLSN | LVKSGEV | AVGSNQDNYQLI | CAVGSNQDNYQLIW |
| KVL-01-075 | 5, 6, 39, 40 | TTLSN | LVKSGEV | AVGSNQENYQLI | CAVGSNQENYQLIW |
| KVL-01-076 | 5, 6, 41, 42 | TTLSN | LVKSGEV | AVGSNQMNYQLI | CAVGSNQMNYQLIW |
| KVL-01-077 | 5, 6, 43, 44 | TTLSN | LVKSGEV | AVGSNQVNYQLI | CAVGSNQVNYQLIW |
| KVL-01-078 | 15, 22, 29, 30 | TTTSN | LEKSGEV | AVGSRDSNYQLV | CAVGSRDSNYQLVW |
| KVL-01-079 | 15, 23, 29, 30 | TTTSN | LGQSGEV | AVGSRDSNYQLV | CAVGSRDSNYQLVW |
| KVL-01-083 | 15, 22, 31, 32 | TTTSN | LEKSGEV | AVGSRDSNYQLI | CAVGSRDSNYQLIW |
| KVL-01-084 | 15, 23, 31, 32 | TTTSN | LGQSGEV | AVGSRDSNYQLI | CAVGSRDSNYQLIW |
| KVL-01-088 | 15, 6, 31, 32 | TTTSN | LVKSGEV | AVGSRDSNYQLI | CAVGSRDSNYQLIW |
| KVL-01-089 | 15, 6, 7, 26 | TTTSN | LVKSGEV | AVGSMDSNYQLI | CAVGSMDSNYQLIW |
| KVL-01-090 | 16, 22, 31, 32 | DASTE | LEKSGEV | AVGSRDSNYQLI | CAVGSRDSNYQLIW |
| KVL-01-094 | 17, 23, 31, 32 | SRSGN | LGQSGEV | AVGSRDSNYQLI | CAVGSRDSNYQLIW |
| KVL-01-097 | 17, 6, 31, 32 | SRSGN | LVKSGEV | AVGSRDSNYQLI | CAVGSRDSNYQLIW |
| KVL-01-105 | 5, 23, 31, 32 | TTLSN | LGQSGEV | AVGSRDSNYQLI | CAVGSRDSNYQLIW |
| KVL-01-108 | 5, 22, 31, 32 | TTLSN | LEKSGEV | AVGSRDSNYQLI | CAVGSRDSNYQLIW |
| KVL-01-110 | 5, 6, 29, 30 | TTLSN | LVKSGEV | AVGSRDSNYQLV | CAVGSRDSNYQLVW |
| KVL-01-111 | 5, 23, 29, 30 | TTLSN | LGQSGEV | AVGSRDSNYQLV | CAVGSRDSNYQLVW |
| KVL-01-112 | 5, 22, 29, 30 | TTLSN | LEKSGEV | AVGSRDSNYQLV | CAVGSRDSNYQLVW |
| KVL-01-117 | 5, 6, 45, 46 | TTLSN | LVKSGEV | AVGSRQSNYQLI | CAVGSRQSNYQLIW |
| KVL-01-119 | 5, 19, 47, 48 | TTLSN | LPKSGEV | AVGSMQSNYQLI | CAVGSMQSNYQLIW |
| KVL-01-123 | 5, 19, 45, 46 | TTLSN | LPKSGEV | AVGSRQSNYQLI | CAVGSRQSNYQLIW |
| KVL-01-124 | 5, 19, 45, 46 | TTLSN | LPKSGEV | AVGSRQSNYQLI | CAVGSRQSNYQLIW |
| KVL-01-125 | 5, 19, 47, 48 | TTLSN | LPKSGEV | AVGSMQSNYQLI | CAVGSMQSNYQLIW |
| KVL-01-126 | 5, 19, 31, 32 | TTLSN | LPKSGEV | AVGSRDSNYQLI | CAVGSRDSNYQLIW |
| KVL-01-128 | 5, 19, 31, 32 | TTLSN | LPKSGEV | AVGSRDSNYQLI | CAVGSRDSNYQLIW |
| KVL-01-129 | 5, 6, 31, 32 | TTLSN | LVKSGEV | AVGSRDSNYQLI | CAVGSRDSNYQLIW |
| KVL-01-145 | 5, 6, 140, 141 | TTLSN | LVKSGEV | AVGSVNTNYQLI | CAVGSVNTNYQLIW |
| KVL-01-149 | 5, 22, 140,141 | TTLSN | LEKSGEV | AVGSVNTNYQLI | CAVGSVNTNYQLIW |
| KVL-01-151 | 5, 6, 140, 141 | TTLSN | LVKSGEV | AVGSVNTNYQLI | CAVGSVNTNYQLIW |
| KVL-01-153 | 5, 6, 43, 44 | TTLSN | LVKSGEV | AVGSNQVNYQLI | CAVGSNQVNYQLIW |

In some embodiments, the TCRa variable region, or portion thereof, may comprise one or more of the CDR sequences recited in the above table. For instance, one of the CDR3a sequences. In another example, the TCRa variable region, or portion thereof, may comprise one of the CDR1a sequences, one of the CDR2a sequences, or one of the CDR3a sequences.

In some embodiments, the TCRa variable region, or portion thereof, comprises a CDR1a, CDR2a, and CDR3a recited in Table 1. In some embodiments, the TCRa variable region, or portion thereof, comprises a CDR1a, CDR2a, and CDR3a recited in the same row of the above Table 1 (i.e. derived from the same clone).

TABLE 2

| | SEQ ID NO | | | | CDR3b |
|---|---|---|---|---|---|
| | (in order left | | | | alternative |
| TCR ID | to right) | CDR1b | CDR2b | CDR3b | definition |
| KVL-01-018 | 12, 13, 14, 57 | MNHEY | SVGEGT | ASSQERTWPYNEQF | CASSQERTWPYNEQFF |
| KVL-01-019 | 12, 13, 14, 57 | MNHEY | SVGEGT | ASSQERTWPYNEQF | CASSQERTWPYNEQFF |
| KVL-01-020 | 12, 13, 14, 57 | MNHEY | SVGEGT | ASSQERTWPYNEQF | CASSQERTWPYNEQFF |
| KVL-01-022 | 12, 13, 14, 57 | MNHEY | SVGEGT | ASSQERTWPYNEQF | CASSQERTWPYNEQFF |
| KVL-01-026 | 12, 13, 14, 57 | MNHEY | SVGEGT | ASSQERTWPYNEQF | CASSQERTWPYNEQFF |
| KVL-01-034 | 12, 13, 14, 57 | MNHEY | SVGEGT | ASSQERTWPYNEQF | CASSQERTWPYNEQFF |
| KVL-01-036 | 12, 13, 14, 57 | MNHEY | SVGEGT | ASSQERTWPYNEQF | CASSQERTWPYNEQFF |
| KVL-01-039 | 12, 13, 14, 57 | MNHEY | SVGEGT | ASSQERTWPYNEQF | CASSQERTWPYNEQFF |
| KVL-01-040 | 12, 13, 14, 57 | MNHEY | SVGEGT | ASSQERTWPYNEQF | CASSQERTWPYNEQFF |
| KVL-01-041 | 12, 13, 14, 57 | MNHEY | SVGEGT | ASSQERTWPYNEQF | CASSQERTWPYNEQFF |
| KVL-01-042 | 12, 13, 14, 57 | MNHEY | SVGEGT | ASSQERTWPYNEQF | CASSQERTWPYNEQFF |
| KVL-01-043 | 12, 13, 14, 57 | MNHEY | SVGEGT | ASSQERTWPYNEQF | CASSQERTWPYNEQFF |
| KVL-01-048 | 12, 13, 58, 59 | MNHEY | SVGEGT | ASSQERTQPYNEQF | CASSQERTQPYNEQFF |
| KVL-01-049 | 12, 13, 58, 59 | MNHEY | SVGEGT | ASSQERTQPYNEQF | CASSQERTQPYNEQFF |
| KVL-01-050 | 12, 13, 58, 59 | MNHEY | SVGEGT | ASSQERTQPYNEQF | CASSQERTQPYNEQFF |
| KVL-01-052 | 12, 13, 58, 59 | MNHEY | SVGEGT | ASSQERTQPYNEQF | CASSQERTQPYNEQFF |
| KVL-01-056 | 12, 13, 58, 59 | MNHEY | SVGEGT | ASSQERTQPYNEQF | CASSQERTQPYNEQFF |
| KVL-01-060 | 12, 13, 58, 59 | MNHEY | SVGEGT | ASSQERTQPYNEQF | CASSQERTQPYNEQFF |
| KVL-01-061 | 12, 13, 58, 59 | MNHEY | SVGEGT | ASSQERTQPYNEQF | CASSQERTQPYNEQFF |
| KVL-01-068 | 12, 13, 14, 57 | MNHEY | SVGEGT | ASSQERTWPYNEQF | CASSQERTWPYNEQFF |
| KVL-01-069 | 12, 13, 14, 57 | MNHEY | SVGEGT | ASSQERTWPYNEQF | CASSQERTWPYNEQFF |
| KVL-01-070 | 12, 13, 14, 57 | MNHEY | SVGEGT | ASSQERTWPYNEQF | CASSQERTWPYNEQFF |
| KVL-01-071 | 12, 13, 14, 57 | MNHEY | SVGEGT | ASSQERTWPYNEQF | CASSQERTWPYNEQFF |
| KVL-01-073 | 12, 13, 58, 59 | MNHEY | SVGEGT | ASSQERTQPYNEQF | CASSQERTQPYNEQFF |
| KVL-01-074 | 12, 13, 58, 59 | MNHEY | SVGEGT | ASSQERTQPYNEQF | CASSQERTQPYNEQFF |
| KVL-01-075 | 12, 13, 58, 59 | MNHEY | SVGEGT | ASSQERTQPYNEQF | CASSQERTQPYNEQFF |
| KVL-01-076 | 12, 13, 58, 59 | MNHEY | SVGEGT | ASSQERTQPYNEQF | CASSQERTQPYNEQFF |
| KVL-01-077 | 12, 13, 58, 59 | MNHEY | SVGEGT | ASSQERTQPYNEQF | CASSQERTQPYNEQFF |
| KVL-01-078 | 12, 13, 14, 57 | MNHEY | SVGEGT | ASSQERTWPYNEQF | CASSQERTWPYNEQFF |
| KVL-01-079 | 12, 13, 14, 57 | MNHEY | SVGEGT | ASSQERTWPYNEQF | CASSQERTWPYNEQFF |
| KVL-01-083 | 12, 13, 14, 57 | MNHEY | SVGEGT | ASSQERTWPYNEQF | CASSQERTWPYNEQFF |
| KVL-01-084 | 12, 13, 14, 57 | MNHEY | SVGEGT | ASSQERTWPYNEQF | CASSQERTWPYNEQFF |
| KVL-01-088 | 12, 13, 14, 57 | MNHEY | SVGEGT | ASSQERTWPYNEQF | CASSQERTWPYNEQFF |
| KVL-01-089 | 12, 13, 14, 57 | MNHEY | SVGEGT | ASSQERTWPYNEQF | CASSQERTWPYNEQFF |
| KVL-01-090 | 12, 13, 14, 57 | MNHEY | SVGEGT | ASSQERTWPYNEQF | CASSQERTWPYNEQFF |
| KVL-01-094 | 12, 13, 14, 57 | MNHEY | SVGEGT | ASSQERTWPYNEQF | CASSQERTWPYNEQFF |
| KVL-01-097 | 12, 13, 14, 57 | MNHEY | SVGEGT | ASSQERTWPYNEQF | CASSQERTWPYNEQFF |
| KVL-01-105 | 12, 13, 14, 57 | MNHEY | SVGEGT | ASSQERTWPYNEQF | CASSQERTWPYNEQFF |
| KVL-01-108 | 12, 13, 14, 57 | MNHEY | SVGEGT | ASSQERTWPYNEQF | CASSQERTWPYNEQFF |
| KVL-01-110 | 12, 13, 14, 57 | MNHEY | SVGEGT | ASSQERTWPYNEQF | CASSQERTWPYNEQFF |
| KVL-01-111 | 12, 13, 14, 57 | MNHEY | SVGEGT | ASSQERTWPYNEQF | CASSQERTWPYNEQFF |
| KVL-01-112 | 12, 13, 14, 57 | MNHEY | SVGEGT | ASSQERTWPYNEQF | CASSQERTWPYNEQFF |
| KVL-01-117 | 12, 13, 14, 57 | MNHEY | SVGEGT | ASSQERTWPYNEQF | CASSQERTWPYNEQFF |
| KVL-01-119 | 12, 13, 14, 57 | MNHEY | SVGEGT | ASSQERTWPYNEQF | CASSQERTWPYNEQFF |
| KVL-01-123 | 12, 13, 58, 59 | MNHEY | SVGEGT | ASSQERTQPYNEQF | CASSQERTQPYNEQFF |
| KVL-01-124 | 12, 13, 14, 57 | MNHEY | SVGEGT | ASSQERTWPYNEQF | CASSQERTWPYNEQFF |
| KVL-01-125 | 12, 13, 14, 57 | MNHEY | SVGEGT | ASSQERTWPYNEQF | CASSQERTWPYNEQFF |
| KVL-01-126 | 12, 13, 14, 57 | MNHEY | SVGEGT | ASSQERTWPYNEQF | CASSQERTWPYNEQFF |
| KVL-01-128 | 12, 56, 14, 57 | MNHEY | SVGEGN | ASSQERTWPYNEQF | CASSQERTWPYNEQFF |
| KVL-01-129 | 12, 13, 60, 61 | MNHEY | SVGEGT | ASSQERTAPYNEQF | CASSQERTAPYNEQFF |
| KVL-01-145 | 12, 13, 14, 57 | MNHEY | SVGEGT | ASSQERTWPYNEQF | CASSQERTWPYNEQFF |
| KVL-01-149 | 12, 13, 14, 57 | MNHEY | SVGEGT | ASSQERTWPYNEQF | CASSQERTWPYNEQFF |
| KVL-01-151 | 12, 13, 14, 57 | MNHEY | SVGEGT | ASSQERTWPYNEQF | CASSQERTWPYNEQFF |
| KVL-01-153 | 12, 13, 58, 59 | MNHEY | SVGEGT | ASSQERTQPYNEQF | CASSQERTQPYNEQFF |

In some embodiments, the TCRb variable region, or portion thereof, may comprise one or more of the CDR sequences recited in the above table. For instance, one of the CDR3b sequences. In another example, the TCRb variable region, or portion thereof, may comprise one of the CDR1b sequences, one of the CDR2b sequences, or one of the CDR3b sequences.

In some embodiments, the TCRb variable region comprises a CDR1b, CDR2b, and CDR3b recited in Table 2. In some embodiments, the TCRb variable region comprises a CDR1b, CDR2b, and CDR3b recited in the same row of the above Table 2 (i.e. derived from the same clone).

The TCR, or antigen-binding portion thereof, may comprise a CDR3a comprising any one of SEQ ID NOs: 7, 26 to 48, 140, and 141 or optionally any one of SEQ ID NOs: 7 and 26 to 48, a CDR1b comprising SEQ ID NO: 12, a CDR2b comprising SEQ ID NO: 13 or SEQ ID NO: 56, and a CDR3b comprising any one of SEQ ID NO: 14 and 57 to 61. The CDR3a, CDR1b, CDR2b, and CDR3b may be associated with the same TCR ID in Tables 1 and 2 (i.e. derived from the same clone).

The TCR, or antigen-binding portion thereof, may comprise a CDR1a comprising any one of SEQ ID NO: 5 and 15 to 17, a CDR2a comprising any one of SEQ ID NO: 6 and 18 to 23, a CDR3a comprising any one of SEQ ID NOs: 7, 26 to 48, 140, and 141 or optionally any one of SEQ ID NOs: 7 and 26 to 48, and a CDR3b comprising any one of SEQ ID NO: 14 and 57 to 61. The CDR1a, CDR2a, CDR3a, and CDR3b may be associated with the same TCR ID in Tables 1 and 2 (i.e. derived from the same clone).

The TCR, or antigen-binding portion thereof, may comprise a CDR1a comprising any one of SEQ ID NO: 5 and 15 to 17, a CDR2a comprising any one of SEQ ID NO: 6 and 18 to 23, a CDR3a comprising any one of SEQ ID NO: 7, 26 to 48, 140, and 141 or optionally any one of SEQ ID NOs: 7 and 26 to 48, a CDR1b comprising SEQ ID NO: 12, a CDR2b comprising SEQ ID NO: 13 or SEQ ID NO: 56, and a CDR3b comprising any one of SEQ ID NO: 14 and 57 to 61. The CDR1a, CDR2a, CDR3a, CDR1b, CDR2b, and CDR3b may be associated with the same TCR ID in Tables 1 and 2 (i.e. derived from the same clone).

In an aspect, the TCR, or antigen-binding portion thereof, may comprise:
(i) a TCRa variable region, or portion thereof, comprising a CDR3a and a TCRb variable region, or portion thereof, comprising a CDR3b; or
(ii) a TCRa variable region, or portion thereof, comprising a CDR3a and a TCRb variable region, or portion thereof, comprising a CDR1b, CDR2b, and CDR3b; or
(iii) a TCRa variable region, or portion thereof, comprising a CDR1a, a CDR2a, and a CDR3a and a TCRb variable region, or portion thereof, comprising a CDR3b; or
(iv) a TCRa variable region, or portion thereof, comprising a CDR1a, a CDR2a, a CDR3a and a TCRb variable region, or portion thereof, comprising a CDR1b, CDR2b, and CDR3b;

wherein the CDR sequences are a set of CDR sequences from one of the clones KVL-01-018, KVL-01-019, KVL-01-020, KVL-01-022, KVL-01-026, KVL-01-034, KVL-01-036, KVL-01-039, KVL-01-040, KVL-01-041, KVL-01-042, KVL-01-043, KVL-01-048, KVL-01-049, KVL-01-050, KVL-01-052, KVL-01-056, KVL-01-060, KVL-01-061, KVL-01-068, KVL-01-069, KVL-01-070, KVL-01-071, KVL-01-073, KVL-01-074, KVL-01-075, KVL-01-076, KVL-01-077, KVL-01-078, KVL-01-079, KVL-01-083, KVL-01-084, KVL-01-088, KVL-01-089, KVL-01-090, KVL-01-094, KVL-01-097, KVL-01-105, KVL-01-108, KVL-01-110, KVL-01-111, KVL-01-112, KVL-01-117, KVL-01-119, KVL-01-123, KVL-01-124, KVL-01-125, KVL-01-126, KVL-01-128, KVL-01-129, KVL-01-145, KVL-01-149, KVL-01-151, or KVL-01-153.

The TCR, or antigen-binding portion thereof, may comprise a CDR3a comprising any one of SEQ ID NOs: 7, 26 to 55, 140, and 141 or optionally any one of SEQ ID NOs: 7 and 26 to 55 and a CDR3b comprising any one of SEQ ID NOs: 14 and 57 to 66, wherein the CDR3a has been grafted into a human TCRa variable region framework and the CDR3b has been grafted into a human TCRb variable region framework. The TCR, or antigen-binding portion thereof, may comprise a CDR3a comprising any one of SEQ ID NOs: 7, 26 to 55, 140, and 141 or optionally any one of SEQ ID NOs: 7 and 26 to 55, a CDR1b comprising SEQ ID NOs 12, a CDR2b comprising SEQ ID NO: 13 or SEQ ID NO: 56, and a CDR3b comprising any one of SEQ ID NOs: 14 and 57 to 66, wherein the CDR3a has been grafted into a human TCRa variable region framework and the CDR1b, CDR2b, and CDR3b have been grafted into a human TCRb variable region framework. The TCR, or antigen-binding portion thereof, may comprise a CDR1a comprising any one of SEQ ID NOs: 5 and 15 to 17, a CDR2a comprising any one of SEQ ID NOs: 6 and 18 to 25, a CDR3a comprising any one of SEQ ID NOs: 7, 26 to 55, 140, and 141 or optionally any one of SEQ ID NOs: 7 and 26 to 55, and a CDR3b comprising any one of SEQ ID NOs: 14 and 57 to 66, wherein the CDR1a, CDR2a, and CDR3a have been grafted into a human TCRa variable region framework and the CDR3b has been grafted into a human TCRb variable region framework. The TCR, or antigen-binding portion thereof, may comprise a CDR1a comprising any one of SEQ ID NOs: 5 and 15 to 17, a CDR2a comprising any one of SEQ ID NOs: 6 and 18 to 25, a CDR3a comprising any one of SEQ ID NOs: 7, 26 to 55, 140, and 141 or optionally any one of SEQ ID NOs: 7 and 26 to 55, a CDR1b comprising SEQ ID NO: 12, a CDR2b comprising SEQ ID NO: 13 or SEQ ID NO: 56, and a CDR3b comprising any one of SEQ ID NOs: 14 and 57 to 66, wherein the CDR1a, CDR2a, and CDR3a have been grafted into a human TCRa variable region framework and the CDR1b, CDR2b, and CDR3b have been grafted into a human TCRb variable region framework. In any such embodiments, the TCR, or antigen-binding portion thereof, does not comprise all six of SEQ ID NOs: 5, 6, 7, 12, 13, and 14. In any such embodiments, the CDRs may be associated with the same TCR ID, i.e. a set of CDRs derived from the same clone.

Any of the CDR sequences disclosed herein may include one, two, or three additional residues at the N-terminal side and/or the C-terminal side. Any of the CDR sequences disclosed herein may include one to ten additional residues at the N-terminal side and/or the C-terminal side. The additional residues may be those contiguous to the recited CDR within the relevant sequence of Table 3 or Table 4, i.e. the CDRs may include a longer sequence from the parent clone. In an example, the CDRs grafted onto the human TCR framework may be according to the CDRs recited in Table 1 and Table 2, and may include one, two, or three additional residues at the N-terminal side and/or the C-terminal side, wherein the additional residues are the contiguous residues of the relevant sequence in Table 3 or Table 4. In any such embodiments, the grafted CDRs may be associated with the same TCR ID, i.e. derived from the same clone.

The TCRa variable region may comprise a first framework region (FRla) of the sequence: GQQVMQIPOYQHVQEGEDFTTYCNSS (SEQ ID NO: 71). The TCRa variable region may comprise a second framework region (FR2a) of the sequence: IOWYKORPGGHPVFLIQ (SEQ ID NO: 72). The TCRa variable region may comprise a third framework region (FR3a) of the sequence: KKQKRLTFOFGEAKKNSSLHITATOTTDVGTYF (SEQ ID NO: 73). The TCRa variable region may comprise a fourth framework region (FR4a) of the sequence: GAGTKLIX₁KP (SEQ ID NO: 74), wherein X₁ is I or V.

The TCRa variable region may comprise a first framework region that is at least 70%, 80%, 85%, 90%, 95%, 97%, 98%, 99%, or 100% identical to SEQ ID NO: 71. The TCRa variable region may comprise a second framework region that is at least 70%, 80%, 85%, 90%, 95%, 97%, 98%, 99%, or 100% identical to SEQ ID NO: 72. The TCRa variable region may comprise a third framework region that is at least 70%, 80%, 85%, 90%, 95%, 97%, 98%, 99%, or 100% identical to SEQ ID NO: 73. The TCRa variable region may comprise a fourth framework region that is at least 70%, 80%, 85%, 90%, 95%, 97%, 98%, 99%, or 100% identical to SEQ ID NO: 74.

The TCRb variable region may comprise a first framework region (FR1b) of the sequence: NAGVTQTPKFRVLKTGQSMTLLCAQD (SEQ ID NO: 75). The TCRb variable region may comprise a second framework region (FR2b) of the sequence: X₁YWYRQDPX₂MGLRLIHY (SEQ ID NO: 76), wherein X₁ is M or L, and X₂ is D or G. The TCRb variable region may comprise a third framework region (FR3b) of the sequence: TX₁KGEVPDGYNVSRLKKONFLLGLESAAPSQTSVYF (SEQ ID NO: 80), wherein X₁ is A or T. The TCRb variable region may comprise a fourth framework region (FR4b) of the sequence: GPGTRLTVL (SEQ ID NO: 81).

In some embodiments, the FR2b sequence may comprise or may be MYWYRQDPDMGLRLIHY (SEQ ID NO: 77), MYWYRQDPGMGLRLIHY (SEQ ID NO: 78), or LYWYRQDPGMGLRLIHY (SEQ ID NO: 79).

The TCRb variable region may comprise a first framework region that is at least 70%, 80%, 85%, 90%, 95%, 97%, 98%, 99%, or 100% identical to SEQ ID NO: 75. The TCRb variable region may comprise a second framework region that is at least 70%, 80%, 85%, 90%, 95%, 97%, 98%, 99%, or 100% identical to any one of SEQ ID NOs: 76 to 79. The TCRb variable region may comprise a third framework region that is at least 70%, 80%, 85%, 90%, 95%, 97%, 98%, 99%, or 100% identical to SEQ ID NO: 80. The TCRb variable region may comprise a fourth framework region that is at least 70%, 80%, 85%, 90%, 95%, 97%, 98%, 99%, or 100% identical to SEQ ID NO: 81.

Any of SEQ ID NOs: 71 to 81 may comprise one, two, three, or four conservative substitutions of a recited sequence. Any of SEQ ID NOs: 71 to 81 may retain a mutation listed in Table 6.

In an aspect, there is provided a TCR, or an antigen-binding portion thereof, comprising:
a TCR-a variable region, or an antigen-binding portion thereof, and a TCR-b variable region, or an antigen-binding portion thereof, wherein
the TCR-a variable region comprises, in order, a sequence at least 70%, 80%, 90%, 95%, or 100% identical to SEQ ID NO: 71, a CDR1a, a sequence at least 70%, 80%, 90%, 95%, or 100% identical to SEQ ID NO: 72, a CDR2a, a sequence at least 70%, 80%, 90%, 95%, or 100% identical to SEQ ID NO: 73, a CDR3a, and a sequence at least 70%, 80%, 90%, 95%, or 100% identical to SEQ ID NO: 74,
   the CDR3a comprises or is according to any one of SEQ ID NOs: 7, 26 to 55, 140, and 141 or
   optionally any one of SEQ ID NOs: 7 and 26 to 55, and
the TCR-b variable region comprises, in order, a sequence at least 70%, 80%, 90%, 95%, or 100% identical to SEQ ID NO: 75, a CDR1b, a sequence at least 70%, 80%, 90%, 95%, or 100% identical to any one of SEQ ID NOs: 76 to 79, a CDR2b, a sequence at least 70%, 80%, 90%, 95%, or 100% identical to SEQ ID NO: 80, a CDR3b, and a sequence at least 70%, 80%, 90%, 95%, or 100% identical to SEQ ID NO: 81,
   the CDR1b comprises or is according to SEQ ID NO: 12,
   the CDR2b comprises or is according to SEQ ID NO: 13 or SEQ ID NO: 56, and
   the CDR3b comprises or is according to any one of SEQ ID NOs: 14 and 57 to 66.

In any such embodiments, the TCR, or antigen-binding portion thereof, does not comprise all six of SEQ ID NO: 5, 6, 7, 12, 13, and 14. In any such embodiments, the CDRs may be associated with the same TCR ID, i.e. a set of CDRs derived from the same clone. In some embodiments, the framework regions have 95%, 97%, 98%, 99%, or 100% identity to the recited sequences. Any of SEQ ID NOs: 71 to 81 may retain a mutation listed in Table 6.

In an aspect, there is provided a TCR, or an antigen-binding portion thereof, comprising:
a TCR-a variable region, or an antigen-binding portion thereof, and a TCR-b variable region, or an antigen-binding portion thereof, wherein
the TCR-a variable region comprises, in order, a sequence at least 70%, 80%, 90%, 95%, or 100% identical to SEQ ID NO: 71, a CDR1a, a sequence at least 70%, 80%, 90%, 95%, or 100% identical to SEQ ID NO: 72, a CDR2a, a sequence at least 70%, 80%, 90%, 95%, or 100% identical to SEQ ID NO: 73, a CDR3a, and a sequence at least 70%, 80%, 90%, 95%, or 100% identical to SEQ ID NO: 74,
   the CDR1a comprises or is according to any one of SEQ ID NOs: 5 and 15-17,
   the CDR2a comprises or is according to any one of SEQ ID NOs: 6 and 18-25, and
   the CDR3a comprises or is according to any one of SEQ ID NOs: 7, 26 to 55, 140 and 141 or optionally any one of SEQ ID NOs: 7 and 26 to 55, and
the TCR-b variable region comprises, in order, a sequence at least 70%, 80%, 90%, 95%, or 100% identical to SEQ ID NO: 75, a CDR1b, a sequence at least 70%, 80%, 90%, 95%, or 100% identical to any one of SEQ ID NOs: 76 to 79, a CDR2b, a sequence at least 70%, 80%, 90%, 95%, or 100% identical to SEQ ID NO: 80, a CDR3b, and a sequence at least 70%, 80%, 90%, 95%, or 100% identical to SEQ ID NO: 81,
   the CDR3b comprises or is according to any one of SEQ ID NOs: 14 and 57 to 66.

In any such embodiments, the TCR, or antigen-binding portion thereof, does not comprise all six of SEQ ID NO: 5, 6, 7, 12, 13, and 14. In any such embodiments, the CDRs may be associated with the same TCR ID, i.e. a set of CDRs derived from the same clone. In some embodiments, the framework regions have 95%, 97%, 98%, 99%, or 100% identity to the recited sequences. Any of SEQ ID NOs: 71 to 81 may retain a mutation listed in Table 6.

In an aspect, there is provided a TCR, or an antigen-binding portion thereof, comprising:
a TCR-a variable region, or an antigen-binding portion thereof, and a TCR-b variable region, or an antigen-binding portion thereof, wherein
the TCR-a variable region comprises, in order, a sequence at least 70%, 80%, 90%, 95%, or 100% identical to SEQ ID NO: 71, a CDR1a, a sequence at least 70%, 80%, 90%, 95%, or 100% identical to SEQ ID NO: 72, a CDR2a, a sequence at least 70%, 80%, 90%, 95%, or 100% identical to SEQ ID NO: 73, a CDR3a, and a sequence at least 70%, 80%, 90%, 95%, or 100% identical to SEQ ID NO: 74,
   the CDR1a comprises or is according to any one of SEQ ID NOs: 5 and 15-17,
   the CDR2a comprises or is according to any one of SEQ ID NOs: 6 and 18-25, and
   the CDR3a comprises or is according to any one of SEQ ID NOs: 7, 26 to 55, 140, and 141 or optionally any one of SEQ ID NOs: 7 and 26 to 55, and
the TCR-b variable region comprises, in order, a sequence at least 70%, 80%, 90%, 95%, or 100% identical to SEQ ID NO: 75, a CDR1b, a sequence at least 70%, 80%, 90%, 95%, or 100% identical to any one of SEQ ID NOs: 76 to 79, a CDR2b, a sequence at least 70%, 80%, 90%, 95%, or 100% identical to SEQ ID NO: 80, a CDR3b, and a sequence at least 70%, 80%, 90%, 95%, or 100% identical to SEQ ID NO: 81,
   the CDR1b comprises or is according to SEQ ID NO: 12,
   the CDR2b comprises or is according to SEQ ID NO: 13 or SEQ ID NO: 56, and
   the CDR3b comprises or is according to any one of SEQ ID NOs: 14 and 57 to 66.

In any such embodiments, the TCR, or antigen-binding portion thereof, does not comprise all six of SEQ ID NO: 5, 6, 7, 12, 13, and 14. In any such embodiments, the CDRs may be associated with the same TCR ID, i.e. a set of CDRs derived from the same clone. In some embodiments, the framework regions have 95%, 97%, 98%, 99%, or 100% identity to the recited sequences. Any of SEQ ID NOs: 71 to 81 may retain a mutation listed in Table 6.

TABLE 3

| | SEQ ID | |
|---|---|---|
| TCR ID | NO | TCR alpha chain-variable region |
| KVL-01-018 | 82 | GQQVMQIPQYQHVQEGEDFTTYCNSSTTLSNIQWYKQRPGGHPVFLIQWVKSGEVKKQK |
| | | RLTFQFGEAKKNSSLHITATQTTDVGTYFCAVGSMDSNYQLIWGAGTKLIIKP |
| KVL-01-019 | 83 | GQQVMQIPQYQHVQEGEDFTTYCNSSTTLSNIQWYKQRPGGHPVFLIQLPKSGEVKKQK |
| | | RLTFQFGEAKKNSSLHITATQTTDVGTYFCAVGSMDSNYQLIWGAGTKLIIKP |
| KVL-01-020 | 84 | GQQVMQIPQYQHVQEGEDFTTYCNSSTTLSNIQWYKQRPGGHPVFLIQLVLSGEVKKQK |
| | | RLTFQFGEAKKNSSLHITATQTTDVGTYFCAVGSMDSNYQLIWGAGTKLIIKP |
| KVL-01-022 | 85 | GQQVMQIPQYQHVQEGEDFTTYCNSSTTLSNIQWYKQRPGGHPVFLIQLVKSPEVKKQK |
| | | RLTFQFGEAKKNSSLHITATQTTDVGTYFCAVGSMDSNYQLIWGAGTKLIIKP |
| KVL-01-026 | 86 | GQQVMQIPQYQHVQEGEDFTTYCNSSTTLSNIQWYKQRPGGHPVFLIQLVKSGEVKKQK |
| | | RLTFQFGEAKKNSSLHITATQTTDVGTYFCAVGSWDSNYQLIWGAGTKLIIKP |
| KVL-01-034 | 87 | GQQVMQIPQYQHVQEGEDFTTYCNSSTTTSNIQWYKQRPGGHPVFLIQLVKSGEVKKQK |
| | | RLTFQFGEAKKNSSLHITATQTTDVGTYFCAVGSRDSNYQLVWGAGTKLIIKP |
| KVL-01-036 | 88 | GQQVMQIPQYQHVQEGEDFTTYCNSSTTLSNIQWYKQRPGGHPVFLIQLVKSGEVKKQK |
| | | RLTFQFGEAKKNSSLHITATQTTDVGTYFCAVGSRDSNYQLIWGAGTKLIVKP |
| KVL-01-039 | 89 | GQQVMQIPQYQHVQEGEDFTTYCNSSTTTSNIQWYKQRPGGHPVFLIQLVKSGEVKKQK |
| | | RLTFQFGEAKKNSSLHITATQTTDVGTYFCAVGSMDSNYQLVWGAGTKLIVKP |
| KVL-01-040 | 90 | GQQVMQIPQYQHVQEGEDFTTYCNSSTTLSNIQWYKQRPGGHPVFLIQLVKSGEVKKQK |
| | | RLTFQFGEAKKNSSLHITATQTTDVGTYFCAVGSRDSNYQLVWGAGTKLIVKP |
| KVL-01-041 | 91 | GQQVMQIPQYQHVQEGEDFTTYCNSSTTTSNIQWYKQRPGGHPVFLIQLVKSGEVKKQK |
| | | RLTFQFGEAKKNSSLHITATQTTDVGTYFCAVGSRDSNYQLIWGAGTKLIVKP |
| KVL-01-042 | 92 | GQQVMQIPQYQHVQEGEDFTTYCNSSTTTSNIQWYKQRPGGHPVFLIQLVKSGEVKKQK |
| | | RLTFQFGEAKKNSSLHITATQTTDVGTYFCAVGSRDSNYQLVWGAGTKLIVKP |
| KVL-01-043 | 93 | GQQVMQIPQYQHVQEGEDFTTYCNSSTTLSNIQWYKQRPGGHPVFLIQLVKSGEVKKQK |
| | | RLTFQFGEAKKNSSLHITATQTTDVGTYFCAVGSRDSNYQLIWGAGTKLIIKP |
| KVL-01-048 | 82 | GQQVMQIPQYQHVQEGEDFTTYCNSSTTLSNIQWYKQRPGGHPVFLIQWVKSGEVKKQK |
| | | RLTFQFGEAKKNSSLHITATQTTDVGTYFCAVGSMDSNYQLIWGAGTKLIIKP |
| KVL-01-049 | 83 | GQQVMQIPQYQHVQEGEDFTTYCNSSTTLSNIQWYKQRPGGHPVFLIQLPKSGEVKKQK |
| | | RLTFQFGEAKKNSSLHITATQTTDVGTYFCAVGSMDSNYQLIWGAGTKLIIKP |
| KVL-01-050 | 84 | GQQVMQIPQYQHVQEGEDFTTYCNSSTTLSNIQWYKQRPGGHPVFLIQLVLSGEVKKQK |
| | | RLTFQFGEAKKNSSLHITATQTTDVGTYFCAVGSMDSNYQLIWGAGTKLIIKP |
| KVL-01-052 | 85 | GQQVMQIPQYQHVQEGEDFTTYCNSSTTLSNIQWYKQRPGGHPVFLIQLVKSPEVKKQK |
| | | RLTFQFGEAKKNSSLHITATQTTDVGTYFCAVGSMDSNYQLIWGAGTKLIIKP |
| KVL-01-056 | 86 | GQQVMQIPQYQHVQEGEDFTTYCNSSTTLSNIQWYKQRPGGHPVFLIQLVKSGEVKKQK |
| | | RLTFQFGEAKKNSSLHITATQTTDVGTYFCAVGSWDSNYQLIWGAGTKLIIKP |
| KVL-01-060 | 3 | GQQVMQIPQYQHVQEGEDFTTYCNSSTTLSNIQWYKQRPGGHPVFLIQLVKSGEVKKQK |
| | | RLTFQFGEAKKNSSLHITATQTTDVGTYFCAVGSMDSNYQLIWGAGTKLIIKP |
| KVL-01-061 | 93 | GQQVMQIPQYQHVQEGEDFTTYCNSSTTLSNIQWYKQRPGGHPVFLIQLVKSGEVKKQK |
| | | RLTFQFGEAKKNSSLHITATQTTDVGTYFCAVGSRDSNYQLIWGAGTKLIIKP |
| KVL-01-068 | 94 | GQQVMQIPQYQHVQEGEDETTYCNSSTTLSNIQWYKQRPGGHPVFLIQLVKSGEVKKQK |
| | | RLTFQFGEAKKNSSLHITATQTTDVGTYFCAVGSNQRNYQLIWGAGTKLIIKP |
| KVL-01-069 | 95 | GQQVMQIPQYQHVQEGEDFTTYCNSSTTLSNIQWYKQRPGGHPVFLIQLVKSGEVKKQK |
| | | RLTFQFGEAKKNSSLHITATQTTDVGTYFCAVGSNQDNYQLIWGAGTKLIIKP |
| KVL-01-070 | 96 | GQQVMQIPQYQHVQEGEDFTTYCNSSTTLSNIQWYKQRPGGHPVFLIQLVKSGEVKKQK |
| | | RLTFQFGEAKKNSSLHITATQTTDVGTYFCAVGSNQENYQLIWGAGTKLIIKP |
| KVL-01-071 | 97 | GQQVMQIPQYQHVQEGEDFTTYCNSSTTLSNIQWYKQRPGGHPVFLIQLVKSGEVKKQK |
| | | RLTFQFGEAKKNSSLHITATQTTDVGTYFCAVGSNQMNYQLIWGAGTKLIIKP |
| KVL-01-073 | 94 | GQQVMQIPQYQHVQEGEDFTTYCNSSTTLSNIQWYKQRPGGHPVFLIQLVKSGEVKKQK |
| | | RLTFQFGEAKKNSSLHITATQTTDVGTYFCAVGSNQRNYQLIWGAGTKLIIKP |
| KVL-01-074 | 95 | GQQVMQIPQYQHVQEGEDFTTYCNSSTTLSNIQWYKQRPGGHPVFLIQLVKSGEVKKQK |
| | | RLTFQFGEAKKNSSLHITATQTTDVGTYFCAVGSNQDNYQLIWGAGTKLIIKP |
| KVL-01-075 | 96 | GQQVMQIPQYQHVQEGEDFTTYCNSSTTLSNIQWYKQRPGGHPVFLIQLVKSGEVKKQK |
| | | RLTFQFGEAKKNSSLHITATQTTDVGTYFCAVGSNQENYQLIWGAGTKLIIKP |
| KVL-01-076 | 97 | GQQVMQIPQYQHVQEGEDFTTYCNSSTTLSNIQWYKQRPGGHPVFLIQLVKSGEVKKQK |
| | | RLTFQFGEAKKNSSLHITATQTTDVGTYFCAVGSNQMNYQLIWGAGTKLIIKP |
| KVL-01-077 | 98 | GQQVMQIPQYQHVQEGEDFTTYCNSSTTLSNIQWYKQRPGGHPVFLIQLVKSGEVKKQK |
| | | RLTFQFGEAKKNSSLHITATQTTDVGTYFCAVGSNQVNYQLIWGAGTKLIIKP |
| KVL-01-078 | 99 | GQQVMQIPQYQHVQEGEDFTTYCNSSTTTSNIQWYKQRPGGHPVFLIQLEKSGEVKKQK |
| | | RLTFQFGEAKKNSSLHITATQTTDVGTYFCAVGSRDSNYQLVWGAGTKLIIKP |
| KVL-01-079 | 100 | GQQVMQIPQYQHVQEGEDFTTYCNSSTTTSNIQWYKQRPGGHPVFLIQLGQSGEVKKQK |
| | | RLTFQFGEAKKNSSLHITATQTTDVGTYFCAVGSRDSNYQLVWGAGTKLIIKP |
| KVL-01-083 | 101 | GQQVMQIPQYQHVQEGEDFTTYCNSSTTTSNIQWYKQRPGGHPVFLIQLEKSGEVKKQK |
| | | RLTFQFGEAKKNSSLHITATQTTDVGTYFCAVGSRDSNYQLIWGAGTKLIIKP |
| KVL-01-084 | 102 | GQQVMQIPQYQHVQEGEDFTTYCNSSTTTSNIQWYKQRPGGHPVFLIQLGQSGEVKKQK |
| | | RLTFQFGEAKKNSSLHITATQTTDVGTYFCAVGSRDSNYQLIWGAGTKLIIKP |
| KVL-01-088 | 103 | GQQVMQIPQYQHVQEGEDFTTYCNSSTTTSNIQWYKQRPGGHPVFLIQLVKSGEVKKQK |
| | | RLTFQFGEAKKNSSLHITATQTTDVGTYFCAVGSRDSNYQLIWGAGTKLIIKP |
| KVL-01-089 | 104 | GQQVMQIPQYQHVQEGEDFTTYCNSSTTTSNIQWYKQRPGGHPVFLIQLVKSGEVKKQK |
| | | RLTFQFGEAKKNSSLHITATQTTDVGTYFCAVGSMDSNYQLIWGAGTKLIIKP |
| KVL-01-090 | 105 | GQQVMQIPQYQHVQEGEDFTTYCNSSDASTEIQWYKQRPGGHPVFLIQLEKSGEVKKQK |
| | | RLTFQFGEAKKNSSLHITATQTTDVGTYFCAVGSRDSNYQLIWGAGTKLIIKP |
| KVL-01-094 | 106 | GQQVMQIPQYQHVQEGEDFTTYCNSSSRSGNIQWYKQRPGGHPVFLIQLGQSGEVKKQK |
| | | RLTFQFGEAKKNSSLHITATQTTDVGTYFCAVGSRDSNYQLIWGAGTKLIIKP |
| KVL-01-097 | 107 | GQQVMQIPQYQHVQEGEDFTTYCNSSSRSGNIQWYKQRPGGHPVFLIQLVKSGEVKKQK |
| | | RLTFQFGEAKKNSSLHITATQTTDVGTYFCAVGSRDSNYQLIWGAGTKLIIKP |
| KVL-01-105 | 108 | GQQVMQIPQYQHVQEGEDFTTYCNSSTTLSNIQWYKQRPGGHPVFLIQLGQSGEVKKQK |
| | | RLTFQFGEAKKNSSLHITATQTTDVGTYFCAVGSRDSNYQLIWGAGTKLIIKP |
| KVL-01-108 | 109 | GQQVMQIPQYQHVQEGEDFTTYCNSSTTLSNIQWYKQRPGGHPVFLIQLEKSGEVKKQK |
| | | RLTFQFGEAKKNSSLHITATQTTDVGTYFCAVGSRDSNYQLIWGAGTKLIIKP |
| KVL-01-110 | 110 | GQQVMQIPQYQHVQEGEDFTTYCNSSTTLSNIQWYKQRPGGHPVFLIQLVKSGEVKKQK |
| | | RLTFQFGEAKKNSSLHITATQTTDVGTYFCAVGSRDSNYQLVWGAGTKLIIKP |
| KVL-01-111 | 111 | GQQVMQIPQYQHVQEGEDFTTYCNSSTTLSNIQWYKQRPGGHPVFLIQLGQSGEVKKQK |
| | | RLTFQFGEAKKNSSLHITATQTTDVGTYFCAVGSRDSNYQLVWGAGTKLIIKP |
| KVL-01-112 | 112 | GQQVMQIPQYQHVQEGEDFTTYCNSSTTLSNIQWYKQRPGGHPVFLIQLEKSGEVKKQK |
| | | RLTFQFGEAKKNSSLHITATQTTDVGTYFCAVGSRDSNYQLVWGAGTKLIIKP |
| KVL-01-117 | 113 | GQQVMQIPQYQHVQEGEDFTTYCNSSTTLSNIQWYKQRPGGHPVFLIQLVKSGEVKKQK |
| | | RLTFQFGEAKKNSSLHITATQTTDVGTYFCAVGSRQSNYQLIWGAGTKLIIKP |
| KVL-01-119 | 114 | GQQVMQIPQYQHVQEGEDFTTYCNSSTTLSNIQWYKQRPGGHPVFLIQLPKSGEVKKQK |
| | | RLTFQFGEAKKNSSLHITATQTTDVGTYFCAVGSMQSNYQLIWGAGTKLIIKP |
| KVL-01-123 | 115 | GQQVMQIPQYQHVQEGEDFTTYCNSSTTLSNIQWYKQRPGGHPVFLIQLPKSGEVKKQK |
| | | RLTFQFGEAKKNSSLHITATQTTDVGTYFCAVGSRQSNYQLIWGAGTKLIIKP |
| KVL-01-124 | 115 | GQQVMQIPQYQHVQEGEDFTTYCNSSTTLSNIQWYKQRPGGHPVFLIQLPKSGEVKKQK |
| | | RLTFQFGEAKKNSSLHITATQTTDVGTYFCAVGSRQSNYQLIWGAGTKLIIKP |
| KVL-01-125 | 114 | GQQVMQIPQYQHVQEGEDFTTYCNSSTTLSNIQWYKQRPGGHPVFLIQLPKSGEVKKQK |
| | | RLTFQFGEAKKNSSLHITATQTTDVGTYFCAVGSMQSNYQLIWGAGTKLIIKP |
| KVL-01-126 | 116 | GQQVMQIPQYQHVQEGEDFTTYCNSSTTLSNIQWYKQRPGGHPVFLIQLPKSGEVKKQK |
| | | RLTFQFGEAKKNSSLHITATQTTDVGTYFCAVGSRDSNYQLIWGAGTKLIIKP |
| KVL-01-128 | 116 | GQQVMQIPQYQHVQEGEDFTTYCNSSTTLSNIQWYKQRPGGHPVFLIQLPKSGEVKKQK |
| | | RLTFQFGEAKKNSSLHITATQTTDVGTYFCAVGSRDSNYQLIWGAGTKLIIKP |
| KVL-01-129 | 93 | GQQVMQIPQYQHVQEGEDFTTYCNSSTTLSNIQWYKQRPGGHPVFLIQLVKSGEVKKQK |
| | | RLTFQFGEAKKNSSLHITATQTTDVGTYFCAVGSRDSNYQLIWGAGTKLIIKP |
| KVL-01-145 | 142 | GQQVMQIPQYQHVQEGEDFTTYCNSSTTLSNIQWYKQRPGGHPVFLIQLVKSGEVKKQK |
| | | RLTFQFGEAKKNSSLHITATQTTDVGTYFCAVGSVNTNYQLIWGAGTKLIIKP |
| KVL-01-149 | 143 | GQQVMQIPQYQHVQEGEDFTTYCNSSTTLSNIQWYKQRPGGHPVFLIQLEKSGEVKKQK |
| | | RLTFQFGEAKKNSSLHITATQTTDVGTYFCAVGSVNTNYQLIWGAGTKLIIKP |
| KVL-01-151 | 142 | GQQVMQIPQYQHVQEGEDFTTYCNSSTTLSNIQWYKQRPGGHPVFLIQLVKSGEVKKQK |
| | | RLTFQFGEAKKNSSLHITATQTTDVGTYFCAVGSVNTNYQLIWGAGTKLIIKP |
| KVL-01-153 | 98 | GQQVMQIPQYQHVQEGEDFTTYCNSSTTLSNIQWYKQRPGGHPVFLIQLVKSGEVKKQK |
| | | RLTFQFGEAKKNSSLHITATQTTDVGTYFCAVGSNQVNYQLIWGAGTKLIIKP |

In an embodiment, the TCR, or antigen-binding portion thereof, comprises an alpha chain variable region comprising or according to any one of the amino acid sequences recited in Table 3.

In some embodiments, the TCR, or antigen-binding portion thereof, comprises any one of SEQ ID NOs: 3, 82 to 116, 142, and 143. The TCR, or antigen-binding portion thereof, may comprise a sequence that is at least 70%, 80%, 85%, 90%, 95%, 97%, 98%, 99%, or 100% identical to any one of SEQ ID NOs: 3, 82 to 116, 142, and 143. In some embodiments, TCR, or antigen-binding portion thereof, comprises a sequence that is at least 70%, 80%, 85%, 90%, 95%, 97%, 98%, 99%, or 100% identical to any one of SEQ ID NOs: 3, 82 to 116, 142, and 143, wherein CDR3a is invariant. The TCR, or antigen-binding portion thereof, may comprise a sequence that is at least 70%, 80%, 85%, 90%, 95%, 97%, 98%, 99%, or 100% identical to any one of SEQ ID NOs: 3, 82 to 116, 142, and 143, wherein CDR1a, CDR2a, and CDR3a are invariant. The CDR sequences may be as recited in Table 1.

The TCR, or antigen-binding portion, may comprise any of the alpha chain mutations listed Table 6. Any of SEQ ID NOs: 82 to 116, 142, and 143, when present with a sequence identity lower than 100%, may retain the mutations associated with the relevant clone in Table 6.

As used herein, a CDR is said to be “invariant” when it is maintained without changes in a sequence that is otherwise not 100% identical to the recited variable region sequence.

The antigen-binding portion of a TCR may comprise a fragment of any one of SEQ ID NOs: 3, 82 to 116, 142, and 143. The antigen-binding portion of a TCR may comprise a fragment that is at least 70%, 80%, 85%, 90%, 95%, 97%, 98%, 99%, or 100% identical to the corresponding portion of any one of SEQ ID NOs: 3, 82 to 116, 142, and 143. The antigen-binding portion of a TCR may comprise a fragment that is at least 70%, 80%, 85%, 90%, 95%, 97%, 98%, 99%, or 100% identical to the corresponding portion of any one of SEQ ID NOs: 3, 82 to 116, 142, and 143, wherein CDR3a is retained and invariant. The antigen-binding portion of a TCR may comprise a fragment that is at least 70%, 80%, 85%, 90%, 95%, 97%, 98%, 99%, or 100% identical to the corresponding portion of any one of SEQ ID NOs: 3, 82 to 116, 142, and 143, wherein CDR1a, CDR2a, and CDR3a are retained and invariant. The CDR sequences may be as recited in Table 1.

The TCR may comprise an alpha chain variable region according to any one of SEQ ID NOs: 3, 82 to 116, 142, and 143, or an antigen-binding portion thereof. The TCR may comprise an alpha chain variable region according to a sequence that is at least 95%, 97%, 98%, 99%, or 100% identical to any one of SEQ ID NOs: 3, 82 to 116, 142, and 143, or an antigen-binding portion thereof. In some embodiments, the TCR comprises an alpha chain variable region according to a sequence that is at least 95%, 97%, 98%, 99%, or 100% identical to any one of SEQ ID NOs: 3, 82 to 116, 142, and 143, wherein CDR3a is invariant, or an antigen-binding portion thereof. The TCR may comprise an alpha chain variable region according to a sequence that is at least 95%, 97%, 98%, 99%, or 100% identical to any one of SEQ ID NOs: 3, 82 to 116, 142, and 143, wherein CDR1a, CDR2a, and CDR3a are invariant, or an antigen-binding portion thereof. The TCR may comprise an alpha chain variable region according to a sequence that is at least 95%, 97%, 98%, 99%, or 100% identical to any one of SEQ ID NOs: 3, 82 to 116, 142, and 143, wherein CDR1a, CDR2a, and CDR3a are invariant, or an antigen-binding portion thereof.

TABLE 4

| | SEQ ID | |
|---|---|---|
| TCR ID | NO | TCR beta chain-variable region |
| KVL-01-018 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-019 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-020 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-022 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-026 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-034 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-036 | 117 | NAGVTQTPKFRVLKTGQSMTLLCAQDMNHEYLYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-039 | 117 | NAGVTQTPKFRVLKTGQSMTLLCAQDMNHEYLYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-040 | 117 | NAGVTQTPKFRVLKTGQSMTLLCAQDMNHEYLYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-041 | 117 | NAGVTQTPKFRVLKTGQSMTLLCAQDMNHEYLYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-042 | 117 | NAGVTQTPKFRVLKTGQSMTLLCAQDMNHEYLYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-043 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-048 | 118 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTQPYNEQFFGPGTRLTVL |
| KVL-01-049 | 118 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTQPYNEQFFGPGTRLTVL |
| KVL-01-050 | 118 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTQPYNEQFFGPGTRLTVL |
| KVL-01-052 | 118 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTQPYNEQFFGPGTRLTVL |
| KVL-01-056 | 118 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTQPYNEQFFGPGTRLTVL |
| KVL-01-060 | 118 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTQPYNEQFFGPGTRLTVL |
| KVL-01-061 | 118 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTQPYNEQFFGPGTRLTVL |
| KVL-01-068 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-069 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-070 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-071 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-073 | 118 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTQPYNEQFFGPGTRLTVL |
| KVL-01-074 | 118 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTQPYNEQFFGPGTRLTVL |
| KVL-01-075 | 118 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTQPYNEQFFGPGTRLTVL |
| KVL-01-076 | 118 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTQPYNEQFFGPGTRLTVL |
| KVL-01-077 | 118 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTQPYNEQFFGPGTRLTVL |
| KVL-01-078 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-079 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-083 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-084 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-088 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-089 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-090 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-094 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-097 | 119 | NAGVTQTPKFRVLKTGQSMTLLCAQDMNHEYMYWYRQDPDMGLRLIHYSVGEGTTTKGE |
| | | VPDGYNVSRLKKQNFLLGLESAAPSQTSVYFCASSQERTWPYNEQFFGPGTRLTVL |
| KVL-01-105 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-108 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-110 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-111 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-112 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-117 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-119 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-123 | 118 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTQPYNEQFFGPGTRLTVL |
| KVL-01-124 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-125 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-126 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-128 | 120 | NAGVTQTPKFRVLKTGQSMTLLCAQDMNHEYMYWYRQDPGMGLRLIHYSVGEGNTAKGE |
| | | VPDGYNVSRLKKQNFLLGLESAAPSQTSVYFCASSQERTWPYNEQFFGPGTRLTVL |
| KVL-01-129 | 121 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTAPYNEQFFGPGTRLTVL |
| KVL-01-145 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-149 | 10 | NAGVTQTPKFRVLKTGQSMTLLCAQDMNHEYMYWYRQDPGMGLRLIHYSVGEGTTAKGE |
| | | VPDGYNVSRLKKQNFLLGLESAAPSQTSVYFCASSQERTWPYNEQFFGPGTRLTVL |
| KVL-01-151 | 119 | NAGVTQTPKFRVLKTGQSMTLLCAQDMNHEYMYWYRQDPDMGLRLIHYSVGEGTTTKGE |
| | | VPDGYNVSRLKKQNFLLGLESAAPSQTSVYFCASSQERTWPYNEQFFGPGTRLTVL |
| KVL-01-153 | 144 | NAGVTQTPKFRVLKTGQSMTLLCAQDMNHEYMYWYRQDPDMGLRLIHYSVGEGTTTKGE |
| | | VPDGYNVSRLKKQNFLLGLESAAPSQTSVYFCASSQERTQPYNEQFFGPGTRLTVL |

In an embodiment, the TCR, or antigen-binding portion thereof, comprises a beta chain variable region comprising or according to any one of the amino acid sequences recited in Table 4.

In some embodiments, the TCR, or antigen-binding portion thereof, comprises any one of SEQ ID NOs: 10, 117 to 121, and 144. The TCR, or antigen-binding portion thereof, may comprise a sequence that is at least 70%, 80%, 85%, 90%, 95%, 97%, 98%, 99%, or 100% identical to any one of SEQ ID NOs: 10, 117 to 121, and 144. In some embodiments, TCR, or antigen-binding portion thereof, comprises a sequence that is at least 70%, 80%, 85%, 90%, 95%, 97%, 98%, 99%, or 100% identical to any one of SEQ ID NOs: 10, 117 to 121, and 144, wherein CDR3b is invariant. The TCR, or antigen-binding portion thereof, may comprise a sequence that is at least 70%, 80%, 85%, 90%, 95%, 97%, 98%, 99%, or 100% identical to any one of SEQ ID NOs: 10, 117 to 121, and 144, wherein CDR1b, CDR2b, and CDR3b are invariant. The CDR sequences may be as recited in Table 2.

The TCR, or antigen-binding portion, may comprise any of the beta chain mutations listed Table 6. Any of SEQ ID NOs: 117 to 121, and 144, when present with a sequence identity lower than 100%, may retain the mutations associated with the relevant clone in Table 6.

The antigen-binding portion of a TCR may comprise a fragment of any one of SEQ ID NOs: 10, 117 to 121, and 144. The antigen-binding portion of a TCR may comprise a fragment that is at least 70%, 80%, 85%, 90%, 95%, 97%, 98%, 99%, or 100% identical to the corresponding portion of any one of SEQ ID NOs: 10, 117 to 121, and 144. The antigen-binding portion of a TCR may comprise a fragment that is at least 70%, 80%, 85%, 90%, 95%, 97%, 98%, 99%, or 100% identical to the corresponding portion of any one of SEQ ID NOs: 10, 117 to 121, and 144, wherein CDR3b is retained and invariant. The antigen-binding portion of a TCR may comprise a fragment that is at least 70%, 80%, 85%, 90%, 95%, 97%, 98%, 99%, or 100% identical to the corresponding portion of any one of SEQ ID NOs: 10, 117 to 121, and 144, wherein CDR1b, CDR2b, and CDR3b are retained and invariant. The CDR sequences may be as recited in Table 2.

The TCR may comprise a beta chain variable region according to any one of SEQ ID NOs: 10, 117 to 121, and 144, or an antigen-binding portion thereof. The TCR may comprise a beta chain variable region according to a sequence that is at least 95%, 97%, 98%, 99%, or 100% identical to any one of SEQ ID NOs: 10, 117 to 121, and 144, or an antigen-binding portion thereof. In some embodiments, the TCR comprises a beta chain variable region according to a sequence that is at least 95%, 97%, 98%, 99%, or 100% identical to any one of SEQ ID NOs: 10, 117 to 121, and 144, wherein CDR3b is invariant, or an antigen-binding portion thereof. The TCR may comprise a beta chain variable region according to a sequence that is at least 95%, 97%, 98%, 99%, or 100% identical to any one of SEQ ID NOs: 10, 117 to 121, and 144, wherein CDR1b, CDR2b, and CDR3b are invariant, or an antigen-binding portion thereof.

The TCR may comprise a sequence recited in Table 3 and a sequence recited in Table 4. The sequence from Table 3 and the sequence from Table 4 may be of the same TCR ID, i.e., derived from the same clone. Antigen-binding portions of said TCRs and of said sequences are also provided.

The TCR may comprise an alpha chain variable region according to any one of SEQ ID NO: 3, 82 to 116, 142, and 143 and a beta chain variable region according to any one of SEQ ID NO: 10, 117 to 121, and 144; or optionally an alpha chain variable region according to any one of SEQ ID NOs: SEQ ID NOs: 3 and 82 to 116 and a beta chain variable region according to any one of SEQ ID NOs: 10 and 117 to 121. The alpha chain variable region and the beta chain variable region may be of the same TCR ID, i.e., derived from the same clone. Antigen-binding portions of said TCRs are also provided. The TCR is not of sequences according to SEQ ID NO: 3 and SEQ ID NO: 10.

The TCR may comprise an alpha chain variable region according to a sequence that is at least 70%, 80%, 85%, 90%, 95%, 97%, 98%, 99%, or 100% identical to any one of SEQ ID NOs: 3, 82 to 116, 142, and 143 and a beta chain variable region according to a sequence that is at least 70%, 80%, 85%, 90%, 95%, 97%, 98%, 99%, or 100% identical to any one of SEQ ID NOs: 10, 117 to 121, and 144. The TCR may comprise an alpha chain variable region according to a sequence that is at least 70%, 80%, 85%, 90%, 95%, 97%, 98%, 99%, or 100% identical to any one of SEQ ID NOs: 3, 82 to 116, 142, and 143 and a beta chain variable region according to a sequence that is at least 70%, 80%, 85%, 90%, 95%, 97%, 98%, 99%, or 100% identical to any one of SEQ ID NOs: 10, 117 to 121, and 144, wherein the alpha chain variable region and the beta chain variable region have the same TCR ID in Tables 3 and 4. The TCR may comprise an alpha chain variable region according to a sequence that is at least 70%, 80%, 85%, 90%, 95%, 97%, 98%, 99%, or 100% identical to any one of SEQ ID NOs: 3, 82 to 116, 142, and 143 and a beta chain variable region according to a sequence that is at least 70%, 80%, 85%, 90%, 95%, 97%, 98%, 99%, or 100% identical to any one of SEQ ID NOs: 10, 117 to 121, and 144, wherein CDR3a and CDR3b are invariant. The TCR may comprise an alpha chain variable region according to a sequence that is at least 70%, 80%, 85%, 90%, 95%, 97%, 98%, 99%, or 100% identical to any one of SEQ ID NOs: 3, 82 to 116, 142, and 143 and a beta chain variable region according to a sequence that is at least 70%, 80%, 85%, 90%, 95%, 97%, 98%, 99%, or 100% identical to any one of SEQ ID NOs: 10, 117 to 121, and 144, wherein the alpha chain variable region and the beta chain variable region have the same TCR ID in Tables 3 and 4 and CDR3a and CDR3b are invariant. The TCR may comprise an alpha chain variable region according to a sequence that is at least 70%, 80%, 85%, 90%, 95%, 97%, 98%, 99%, or 100% identical to any one of SEQ ID NOs: 3, 82 to 116, 142, and 143 and a beta chain variable region according to a sequence that is at least 70%, 80%, 85%, 90%, 95%, 97%, 98%, 99%, or 100% identical to any one of SEQ ID NOs: 10, 117 to 121, and 144, wherein CDR3a, CDR1b, CDR2b, and/or CDR3b are invariant. The TCR may comprise an alpha chain variable region according to a sequence that is at least 70%, 80%, 85%, 90%, 95%, 97%, 98%, 99%, or 100% identical to any one of SEQ ID NOs: 3, 82 to 116, 142, and 143 and a beta chain variable region according to a sequence that is at least 70%, 80%, 85%, 90%, 95%, 97%, 98%, 99%, or 100% identical to any one of SEQ ID NOs: 10, 117 to 121, and 144, wherein the alpha chain variable region and the beta chain variable region have the same TCR ID in Tables 3 and 4 and CDR3a, CDR1b, CDR2b, and/or CDR3b are invariant. The TCR may comprise an alpha chain variable region according to a sequence that is at least 70%, 80%, 85%, 90%, 95%, 97%, 98%, 99%, or 100% identical to any one of SEQ ID NOs: 3, 82 to 116, 142, and 143 and a beta chain variable region according to a sequence that is at least 70%, 80%, 85%, 90%, 95%, 97%, 98%, 99%, or 100% identical to any one of SEQ ID NOs: 10, 117 to 121, and 144, wherein CDR1a, CDR2a, CDR3a, and/or CDR3b are invariant. The TCR may comprise an alpha chain variable region according to a sequence that is at least 70%, 80%, 85%, 90%, 95%, 97%, 98%, 99%, or 100% identical to any one of SEQ ID NOs: 3, 82 to 116, 142, and 143 and a beta chain variable region according to a sequence that is at least 70%, 80%, 85%, 90%, 95%, 97%, 98%, 99%, or 100% identical to any one of SEQ ID NOs: 10, 117 to 121, and 144, wherein the alpha chain variable region and the beta chain variable region have the same TCR ID in Tables 3 and 4 and CDR1a, CDR2a, CDR3a, and/or CDR3b are invariant. The TCR may comprise an alpha chain variable region according to a sequence that is at least 70%, 80%, 85%, 90%, 95%, 97%, 98%, 99%, or 100% identical to any one of SEQ ID NOs: 3, 82 to 116, 142, and 143 and a beta chain variable region according to a sequence that is at least 70%, 80%, 85%, 90%, 95%, 97%, 98%, 99%, or 100% identical to any one of SEQ ID NOs: 10, 117 to 121, and 144, wherein CDR1a, CDR2a, CDR3a, CDR1b, CDR2b, and/or CDR3b are invariant. The TCR may comprise an alpha chain variable region according to a sequence that is at least 70%, 80%, 85%, 90%, 95%, 97%, 98%, 99%, or 100% identical to any one of SEQ ID NOs: 3, 82 to 116, 142, and 143 and a beta chain variable region according to a sequence that is at least 70%, 80%, 85%, 90%, 95%, 97%, 98%, 99%, or 100% identical to any one of SEQ ID NOs: 10, 117 to 121, and 144, wherein the alpha chain variable region and the beta chain variable region have the same TCR ID in Tables 3 and 4 and CDR1a, CDR2a, CDR3a, CDR1b, CDR2b, and/or CDR3b are invariant. Antigen-binding portions of all of said TCRs are also provided. The TCR does not comprise both SEQ ID NO: 3 and SEQ ID NO: 10. In any of the statements in this paragraph the combination may be one of SEQ ID NOs: 3 and 82 to 116 with one of SEQ ID NOs: 10 and 117 to 121.

The TCR, or antigen-binding portion, may comprise any of the alpha chain mutations and/or beta chain mutations listed Table 6. In an example, the alpha chain mutations and the beta chain mutations may be from the same row of Table 6. Any of SEQ ID NOs: 82 to 121 and 142 to 144, when present with a sequence identity lower than 100%, may retain the mutations associated with the relevant clone in Table 6.

The TCR may comprise an alpha chain variable region according to a sequence that is at least 95%, 97%, 98%, 99%, or 100% identical to any one of SEQ ID NOs: 3, 82 to 116, 142, and 143 and a beta chain variable region according to a sequence that is at least 95%, 97%, 98%, 99%, or 100% identical to any one of SEQ ID NOs: 10, 117 to 121, and 144. The TCR may comprise an alpha chain variable region according to a sequence that is at least 95%, 97%, 98%, 99%, or 100% identical to any one of SEQ ID NOs: 3, 82 to 116, 142, and 143 and a beta chain variable region according to a sequence that is at least 95%, 97%, 98%, 99%, or 100% identical to any one of SEQ ID NOs: 10, 117 to 121, and 144, wherein the alpha chain variable region and the beta chain variable region have the same TCR ID in Tables 3 and 4. The TCR may comprise an alpha chain variable region according to a sequence that is at least 95%, 97%, 98%, 99%, or 100% identical to any one of SEQ ID NOs: 3, 82 to 116, 142, and 143 and a beta chain variable region according to a sequence that is at least 95%, 97%, 98%, 99%, or 100% identical to any one of SEQ ID NOs: 10, 117 to 121, and 144, wherein CDR3a and CDR3b are invariant. The TCR may comprise an alpha chain variable region according to a sequence that is at least 95%, 97%, 98%, 99%, or 100% identical to any one of SEQ ID NOs: 3, 82 to 116, 142, and 143 and a beta chain variable region according to a sequence that is at least 95%, 97%, 98%, 99%, or 100% identical to any one of SEQ ID NOs: 10, 117 to 121, and 144, wherein the alpha chain variable region and the beta chain variable region have the same TCR ID in Tables 3 and 4 and CDR3a and CDR3b are invariant. The TCR may comprise an alpha chain variable region according to a sequence that is at least 95%, 97%, 98%, 99%, or 100% identical to any one of SEQ ID NOs: 3, 82 to 116, 142, and 143 and a beta chain variable region according to a sequence that is at least 95%, 97%, 98%, 99%, or 100% identical to any one of SEQ ID NOs: 10, 117 to 121, and 144, wherein CDR3a, CDR1b, CDR2b, and/or CDR3b are invariant. The TCR may comprise an alpha chain variable region according to a sequence that is at least 95%, 97%, 98%, 99%, or 100% identical to any one of SEQ ID NOs: 3, 82 to 116, 142, and 143 and a beta chain variable region according to a sequence that is at least 95%, 97%, 98%, 99%, or 100% identical to any one of SEQ ID NOs: 10, 117 to 121, and 144, wherein the alpha chain variable region and the beta chain variable region have the same TCR ID in Tables 3 and 4 and CDR3a, CDR1b, CDR2b, and/or CDR3b are invariant. The TCR may comprise an alpha chain variable region according to a sequence that is at least 95%, 97%, 98%, 99%, or 100% identical to any one of SEQ ID NOs: 3, 82 to 116, 142, and 143 and a beta chain variable region according to a sequence that is at least 95%, 97%, 98%, 99%, or 100% identical to any one of SEQ ID NOs: 10, 117 to 121, and 144, wherein CDR1a, CDR2a, CDR3a, and/or CDR3b are invariant. The TCR may comprise an alpha chain variable region according to a sequence that is at least 95%, 97%, 98%, 99%, or 100% identical to any one of SEQ ID NOs: 3, 82 to 116, 142, and 143 and a beta chain variable region according to a sequence that is at least 95%, 97%, 98%, 99%, or 100% identical to any one of SEQ ID NOs: 10, 117 to 121, and 144, wherein the alpha chain variable region and the beta chain variable region have the same TCR ID in Tables 3 and 4 and CDR1a, CDR2a, CDR3a, and/or CDR3b are invariant. The TCR may comprise an alpha chain variable region according to a sequence that is at least 95%, 97%, 98%, 99%, or 100% identical to any one of SEQ ID NOs: 3, 82 to 116, 142, and 143 and a beta chain variable region according to a sequence that is at least 95%, 97%, 98%, 99%, or 100% identical to any one of SEQ ID NOs: 10, 117 to 121, and 144, wherein CDR1a, CDR2a, CDR3a, CDR1b, CDR2b, and/or CDR3b are invariant. The TCR may comprise an alpha chain variable region according to a sequence that is at least 95%, 97%, 98%, 99%, or 100% identical to any one of SEQ ID NOs: 3, 82 to 116, 142, and 143 and a beta chain variable region according to a sequence that is at least 95%, 97%, 98%, 99%, or 100% identical to any one of SEQ ID NOs: 10, 117 to 121, and 144, wherein the alpha chain variable region and the beta chain variable region have the same TCR ID in Tables 3 and 4 and CDR1a, CDR2a, CDR3a, CDR1b, CDR2b, and/or CDR3b are invariant. Antigen-binding portions of said TCRs are also provided. The TCR does not comprise both SEQ ID NO: 3 and SEQ ID NO: 10. In any of the statements in this paragraph the combination may be one of SEQ ID NOs: 3 and 82 to 116 with one of SEQ ID NOs: 10 and 117 to 121.

The TCRs, or antigen-portions thereof, are capable of binding specifically to a peptide of the sequence KVLEHVVRV (SEQ ID NO: 122) presented by an MHC class I molecule. The MHC class I molecule may be HLA-A and, in particular, HLA-A*0201. Thus, the TCR, or antigen-binding portion, may be capable of binding specifically to a peptide of the sequence KVLEHVVRV (SEQ ID NO: 122) presented by HLA-A*0201.

It is known in the art that TCRs specific for peptides presented by certain HLA subtypes may also recognise the target peptide presented in the context of other HLA subtypes, for instance related or similar subtypes.

Hence, a TCR, or an antigen-binding portion thereof, as disclosed herein may be capable of binding specifically to a peptide of the sequence KVLEHVVRV (SEQ ID NO: 122) presented by HLA-A*0201. This is applicable to all TCRs and antigen-binding portions described herein.

In an aspect, there is provided a TCR, or antigen-binding portion thereof, capable of binding specifically to a peptide according to SEQ ID NO: 122 presented by HLA-A*0201, wherein the TCR comprises:
(i) a TCRa variable region, or portion thereof, comprising a CDR3a and a TCRb variable region, or portion thereof, comprising a CDR3b; or
(ii) a TCRa variable region, or portion thereof, comprising a CDR3a and a TCRb variable region, or portion thereof, comprising a CDR1b, CDR2b, and CDR3b; or
(iii) a TCRa variable region, or portion thereof, comprising a CDR1a, a CDR2a, and a CDR3a and a TCRb variable region, or portion thereof, comprising a CDR3b; or
(iv) a TCRa variable region, or portion thereof, comprising a CDR1a, a CDR2a, a CDR3a and a TCRb variable region, or portion thereof, comprising a CDR1b, CDR2b, and CDR3b;

wherein the CDR sequences are a set of CDR sequences from one of the clones KVL-01-018, KVL-01-019, KVL-01-020, KVL-01-022, KVL-01-026, KVL-01-034, KVL-01-036, KVL-01-039, KVL-01-040, KVL-01-041, KVL-01-042, KVL-01-043, KVL-01-048, KVL-01-049, KVL-01-050, KVL-01-052, KVL-01-056, KVL-01-060, KVL-01-061, KVL-01-068, KVL-01-069, KVL-01-070, KVL-01-071, KVL-01-073, KVL-01-074, KVL-01-075, KVL-01-076, KVL-01-077, KVL-01-078, KVL-01-079, KVL-01-083, KVL-01-084, KVL-01-088, KVL-01-089, KVL-01-090, KVL-01-094, KVL-01-097, KVL-01-105, KVL-01-108, KVL-01-110, KVL-01-111, KVL-01-112, KVL-01-117, KVL-01-119, KVL-01-123, KVL-01-124, KVL-01-125, KVL-01-126, KVL-01-128, KVL-01-129, KVL-01-145, KVL-01-149, KVL-01-151, or KVL-01-153; and

optionally wherein the TCRa variable region is at least 70%, 80%, 85%, 90%, 95%, 97%, 98%, 99%, or 100% identical, or 95%, 97%, 98%, 99%, or 100% identical, to the TCRa variable region of the clone from which the set of CDR sequences are derived, and

optionally the TCRb variable region is at least 70%, 80%, 85%, 90%, 95%, 97%, 98%, 99%, or 100% identical, or 95%, 97%, 98%, 99%, or 100% identical, to the TCRb variable region of the clone from which the set of CDR sequences are derived. Optionally, the mutations listed in Table 6 may be retained.

The TCRs disclosed herein are capable of specifically binding to a target peptide presented in the context of an MHC class I molecule. As used herein, “specifically binding” means that the TCR is able to bind to the peptide-MHCI complex wherein the affinity of the binding is affected by the sequence of the peptide. The TCRs would not strongly bind to a peptide-MHCI where the peptide is a scrambled peptide presented via the same MHC class I. A TCR may specifically bind to more than one peptide, for instance where minor or conservative changes are made to a target peptide.

In an embodiment, the TCR, or antigen-binding portion thereof, has a low cross-reactivity to a panel of peptides similar to KVLEHVVRV (SEQ ID NO: 122). In particular, the TCR, or antigen-binding portion thereof, may have low cross-reactivity to naturally occurring peptides within the human peptidome.

In some embodiments, the TCR, or antigen-binding portion thereof, has a Kd less than or equal to 200 μM, 175 μM, 143 μM, 100 μM, 75 μM, 50 μM, 25 μM, 20 μM, 15 μM, 10 μM, 5 μM, or 2 μM. The K_{d} may be calculated by surface plasmon resonance (SPR) and the target may be peptide-HLA monomers. The peptide-HLA monomers are immobilised to the sensor chip (e.g. by the use of biotin and streptavidin). Equilibrium binding constants may be determined by flowing serial dilutions of test TCRs at 10 μl/minute for 60 seconds over flow cells loaded with between 50 and 600 response units of peptide-HLA complex. Specific responses may be determined by subtracting the non-specific response of a control flow cell (e.g. immobilised with only streptavidin) from the response of a flow cell loaded with relevant peptide-HLA. The K_{d} value may be determined by nonlinear curve fitting and the binding isotherm equation equivalent to the Langmuir isotherm for the absorption of a gas onto a surface. Further details are provided in the Examples section herein. The method set out in the Examples may be used to determine the Kd of embodiments herein.

In some embodiments, the TCR, or antigen-binding portion thereof, has an EC₅₀ of less than or equal to 500,000 μM, 10,000 μM, 500 μM, 2500 μM, 2000 μM, 1500 μM, 1161 μM, 1000 μM, 500 μM, 400 μM, 300 μM, 200 μM, 150 μM, 100 μM, 75 μM, 50 μM, 40 μM, 30 μM, 25 μM, 20 μM, 15 μM, or 10 μM. The EC₅₀ may be calculated by the transduction of PBMCs with said TCRs or portions, followed by the measurement of reactivity against antigen-presenting cells (APCs) pulsed with different concentrations of a peptide with the sequence KVLEHVVRV (SEQ ID NO: 122) in an IFNγ ELISPOT assay. For more information, see Example 6.

In some embodiments, T cells or NK cells expressing the TCR, or antigen-binding portion thereof, are responsive to cell lines expressing a peptide with the sequence KVLEHVVRV (SEQ ID NO: 122) in the correct MHC context. For instance, the cell lines NCI-H1703 or C33A. In some instances, the T cells or NK cells are more responsive to C33A cells than a reference T cell or NK cell expressing the TCR described in Example 1 as the parent/wild type TCR. In some embodiments, the cells are more than 1.5 fold, 2 fold, 3 fold, 4 fold, 5 fold, 10 fold, 11 fold, or 12 fold more responsive to C33A cells compared to reference cells expressing the parent TCR. In some embodiments, the cells are more than 1.1 fold, 1.2 fold, 1.3 fold, 1.5 fold, 1.75 fold, 2 fold, 2.2 fold, or 2.3 fold more responsive to NCI-H1703 cells compared to reference cells expressing the parent TCR.

The term “T cell receptor” or “TCR”, as used herein, refers to class of molecules that are members of the immunoglobulin superfamily. Functional TCRs are capable of binding to peptide-MHC complexes and are specific for a peptide or a limited range of peptides presented by particular MHC complexes. Intact TCRs comprise a TCR variable region, a TCR constant region, a TCR transmembrane region, and a TCR cytoplasmic tail. The molecules of the present disclosure comprise a TCR variable region, or antigen-binding portion thereof. The molecules of the present disclosure may also comprise a TCR constant region or portion thereof, optionally a transmembrane region, and optionally a cytoplasmic tail. Thus, the molecules of the present disclosure may be intact TCRs or may be portions of intact TCRs.

The TCR, or antigen-binding portion thereof, may be present as a single polypeptide chain. For instance, a single chain comprising a TCR-a variable region and a TCR-b variable region. The TCR-a chain and the TCR-b chain may be connected via a peptide linker. The TCR may be a single chain TCR (e.g. as disclosed in Li et al. TCR extracellular domain genetically linked to CD28, 2B4/41BB and DAP10/CD3ζ-engineered NK cells mediates antitumor effects, Cancer Immunology, Immunotherapy https://doi.org/10.1007/s00262-022-03275-5). Further examples of TCR formats are provided in Oh et al. (Nature Scientific Reports|(2019) 9:17291|https://doi.org/10.1038/s41598-019-53756-4).

Alternatively, the TCR, or antigen-binding portion thereof, may comprise two or more polypeptide chains, for instance one comprising a TCR-a variable region and one comprising a TCR-b variable region. The TCR-a chain and the TCR-b chain may be linked by one or more disulphide bond, non-covalent linkage (such as Van der Waals forces and/or hydrogen bonds), and/or one or more covalent bond (such as a peptide bond).

The TCR, or antigen-binding portion thereof, may be part of one or more longer polypeptide. For instance, the TCR, or antigen-binding portion thereof, may be part of one or more fusion protein comprising other domains or proteins. Examples include CD3 molecules or portions thereof. In other examples, the TCR, or antigen-binding portion thereof, may be a part of a chimeric antigen receptor (CAR), and hence impart antigen-specificity to said receptor. The TCR, or antigen-binding portion thereof, may be in addition to or a replacement for the single-chain variable fragment (scFv) of a CAR.

The TCR, or antigen-binding portion thereof, may be part of a bispecific or bifunctional molecule. For instance, the TCR, or antigen-binding portion thereof, may be part of a complex or fusion protein with at least one other antigen-binding protein, such as a TCR or antibody portion, such as an scFv. The second antigen-binding portion may have specificity for an immune cell, for instance it may be specific for a T cell marker and hence capable of recruiting T cells to MAGE-A4 expressing cells.

The TCR, or antigen-binding portion thereof, may be part of the soluble molecule or composition. The TCR may be a soluble TCR.

The TCR may comprise a TCRa variable region and a TCRb variable region. The TCR may comprise an alpha chain constant region and/or a beta chain constant region. In some embodiments, the TCR comprises a TCRa variable region, a TCRb variable region, and an alpha chain constant region. In some embodiments, the TCR comprises a TCRa variable region, a TCRb variable region, and a beta chain constant region. In some embodiments, the TCR comprises a TCRa variable region, a TCRb variable region, an alpha chain constant region, and a beta chain constant region. Thus, the TCR may comprise four domains, three domains, or two domains of a complete TCR. The TCR may be a single chain TCR comprising four, three, or two domains. In some examples, the alpha and/or beta constant regions are human or derived from human sequences.

The TCR may be chimeric in that the variable regions and constant region or regions are not associated with each other in nature. For instance, the variable regions may include sequence from one species and the constant region or regions may include sequence from a different species. In an example, the constant domains may be murine or derived from murine constant domains. The murine constant domain or domains may be connected to human variable regions. The alpha and/or beta constant regions may be derived from human sequences wherein only some residues have been exchanged for the corresponding residues in a mouse sequence (referred to as “minimal murinization”).

The TCR may comprise intervening sequences between the domains that are not found in naturally occurring TCRs. For instance, linkers between the domains, target sites for antibodies, hinge sequences, transmembrane sequences, and the like. For instance, the TCR may comprise an epitope for an antibody, to enable the identification or elimination or cells expressing said TCR. Such features may be positioned anywhere within the TCR, as long as function is not affected. The TCR may be an engineered TCR.

The TCR, or antigen-binding portion thereof, may include stabilising mutations. For instance, the transmembrane regions of the alpha and beta chains may include mutations favouring the correct TCR chain pairing. Examples of suitable mutations are disclosed in WO2016071343 (herein incorporated by reference). The TCR, or antigen-binding portion thereof, may include mutations that enhance surface expression and functional avidity. Examples of suitable mutations are disclosed in Haga-Friedman et al. (J Immunol 2012; 188:5538-5546, herein incorporated by reference).

An “antigen-binding portion” of a TCR refers to one or more fragments of an intact TCR that retain the ability to bind to the same peptide-MHC complex. A particular peptide-MHC complex may be referred to as the “antigen” for a particular TCR. In the context of the present invention, the antigen-binding portions retain the ability to bind to a peptide derived from MAGE-A4 when presented by an appropriate MHC class I molecule. For instance, the antigen-binding portion of the TCR is capable of binding to a peptide of the sequence KVLEHVVRV (SEQ ID NO: 122) when presented by HLA-A*0201. An antigen-binding portion retains the CDR3 of the respective chain. The antigen-binding portion may also retain the CDR1 and/or the CDR2 of the respective chain. An antigen-binding portion of a TCR may retain CDR3a and/or CDR3b. The antigen-binding portion of a TCR may retain CDR3a, CDR1b, CDR2b, and CDR3b. The antigen-binding portion of a TCR may retain CDR1a, CRD2a, CDR3a, and CDR3b. The antigen-binding portion of a TCR may retain CDR1a, CDR2a, CDR3a, CDR1b, CDR2b, and CDR3b.

A TCR, or antigen-binding portion thereof, disclosed herein may comprise SEQ ID NO: 4. The TCR, or antigen-binding portion thereof, may comprise a sequence that is least 70%, 80%, 85%, 90%, 95%, 97%, 98%, 99%, or 100% identical to SEQ ID NO: 4. The TCR, or antigen-binding portion thereof, may comprise a sequence that is least 95%, 97%, 98%, 99%, or 100% identical to SEQ ID NO: 4.

In some embodiments, the TCR, or antigen-binding portion thereof, comprises SEQ ID NO: 11, 138, or 139. The TCR, or antigen-binding portion thereof, may comprise a sequence that is least 70%, 80%, 85%, 90%, 95%, 97%, 98%, 99%, or 100% identical to any one of SEQ ID NOs: 11, 138, and 139. The TCR, or antigen-binding portion thereof, may comprise a sequence that is least 95%, 97%, 98%, 99%, or 100% identical to any one of SEQ ID NOs: 11, 138, and 139. In a particular embodiment, the TCR, or antigen-binding portion thereof, comprises SEQ ID NO: 11.

EDLNKVFPPEVAVFEPSEAEISHTQKATLVCLATGFFPDHVELSWWVNGKEVHSGVSTDPQPLKEQPALN
DSRYCLSSRLRVSATFWQNPRNHFRCQVQFYGLSENDEWTQDRAKPVTQIVSAEAWGRADCGFTSVSY
QQGVLSATILYEILLGKATLYAVLVSALVLMAMVKRKDF (SEQ ID NO: 138-example of a beta chain
constant region sequence)
EDLKNVFPPEVAVFEPSEAEISHTQKATLVCLATGFYPDHVELSWWVNGKEVHSGVSTDPQPLKEQPAL
NDSRYCLSSRLRVSATFWQNPRNHFRCQVQFYGLSENDEWTQDRAKPVTQIVSAEAWGRADCGFTSES
YQQGVLSATILYEILLGKATLYAVLVSALVLMAMVKRKDSRG (SEQ ID NO: 139-example of a beta
chain constant region sequence)

In some embodiments, the TCR, or antigen-binding portion thereof, comprises SEQ ID NO: 4 and any one of SEQ ID NOs: 11, 138, and 139. The TCR, or antigen-binding portion thereof, may comprise a sequence that is least 70%, 80%, 85%, 90%, 95%, 97%, 98%, 99%, or 100% identical to SEQ ID NO: 4 and a sequence that is least 70%, 80%, 85%, 90%, 95%, 97%, 98%, 99%, or 100% identical to any one of SEQ ID NOs: 11, 138, and 139. The TCR, or antigen-binding portion thereof, may comprise a sequence that is least 95%, 97%, 98%, 99%, or 100% identical to SEQ ID NO: 4 and a sequence that is least 95%, 97%, 98%, 99%, or 100% identical to any one of SEQ ID NOs: 11, 138, and 139. The TCR, or antigen-binding portion thereof, may comprise a sequence that is least 70%, 80%, 85%, 90%, 95%, 97%, 98%, 99%, or 100% identical to SEQ ID NO: 4 and a sequence that is least 70%, 80%, 85%, 90%, 95%, 97%, 98%, 99%, or 100% identical to SEQ ID NO: 11.

The construct encoding the TCR, or antigen-binding portion thereof, may encode a signal peptide or signal peptides. However, in some examples the signal peptide or peptides may be cleaved or removed during expression of the TCR or antigen-binding portion thereof.

The TCRa chain may comprise a signal peptide according to the sequence SEQ ID NO: 2. The TCRb chain may comprise a signal peptide according to the sequence SEQ ID NO: 9.

The TCRa chain may comprise a TCRa variable region according to any one of SEQ ID NOs: 82 to 116, 142, and 143, a signal peptide according to the sequence SEQ ID NO: 2, and a constant region according to SEQ ID NO: 4. The TCRb chain may comprise a TCRb variable region according to any one of SEQ ID NOs: 117 to 121 and 144, a signal peptide according to the sequence SEQ ID NO: 9, and a constant region according to any one of SEQ ID NOs: 11, 138, and 139.

As used herein the term “conservative substitution” refers to replacement of an amino acid with another amino acid which does not significantly deleteriously change the functional activity. Conservative substitutions may be those described as such by Dayhoff in “The Atlas of Protein Sequence and Structure. Vol. 5”, Natl. Biomedical Research. For example, a conservative substitution may be the exchange for a residue from one of the following groups for another residue within the same group:
Group 1: alanine, proline, glycine, asparagine, serine, threonine;
Group 2: cysteine, serine, tyrosine, threonine;
Group 3: valine, isoleucine, leucine, methionine, alanine, phenylalanine;
Group 4: lysine, arginine, histidine;
Group 5: phenylalanine, tyrosine, tryptophan, histidine; and
Group 6: aspartic acid, glutamic acid.

In another example, a conservative amino acid substitution may be selected from: T to A, A to I, T to V, A to M, T to I, A to V, T to G, and/or T to S.

In a further example, a conservative amino acid substitution may include the substitution of an amino acid by another amino acid of the same class, for example, (1) nonpolar: Ala, Val, Leu, Ile, Pro, Met, Phe, Trp; (2) uncharged polar: Gly, Ser, Thr, Cys, Tyr, Asn, Gln; (3) acidic: Asp, Glu; and (4) basic: Lys, Arg, His. Other conservative amino acid substitutions may also be made as follows: (1) aromatic: Phe, Tyr, His; (2) proton donor: Asn, Gln, Lys, Arg, His, Trp; and (3) proton acceptor: Glu, Asp, Thr, Ser, Tyr, Asn, Gln.

In another example, conservative substitutions may be made in accordance with the following:
Ala for Ser, Gly, or Cys
Arg for Lys, Gln, or His
Asn for Gln, His, Glu, or Asp
Asp for Glu, Asn, or Gln
Cys for Ser, Met, or Thr
Gln for Asn, Lys, Glu, Asp, or Arg
Glu for Asp, Asn, or Gln
Gly for Pro, Ala, or Ser
His for Asn, Gln, or Lys
Ile for Leu, Val, Met, or Ala
Leu for Ile, Val, Met, or Ala
Lys for Arg, Gln, or His
Met for Leu, Ile, Val, Ala, or Phe
Phe for Met, Leu, Tyr, Trp, or His
Ser for Thr, Cys, or Ala
Thr for Ser, Val, or Ala
Trp for Tyr, or Phe
Tyr for Trp, Phe, or His
Val for Ile, Leu, Met, Ala, or Thr

A “non-conservative” substitution is one that is not conservative, as defined herein. A “naturally occurring” amino acid is one that forms part of proteins or peptides in naturally occurring organisms. A “canonical” amino acid is one of the 20 amino acids that form proteins in humans.

The TCR, or antigen-binding portion thereof, may contain glycosylation sites and may be glycosylated.

The TCR, or antigen-binding portion of the present disclosure may be isolated. The term “isolated” refers to a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

The present invention encompasses modifications to the amino acid sequence of the TCR molecule or antigen-binding portion thereof as defined herein. For example, the invention includes TCR molecules and corresponding antigen-binding portions thereof comprising functionally equivalent variable regions and CDRs which do not significantly affect their properties as well as variants which have enhanced or decreased activity and/or affinity. For example, the amino acid sequence may be mutated to obtain a TCR with the desired binding affinity to a peptide according to SEQ ID NO: 122 presented by a relevant MHC molecule. Insertions that include amino- and/or carboxyl-terminal fusions ranging in length from one residue to polypeptides containing a hundred or more residues, as well as intrasequence insertions of single or multiple amino acid residues, are envisaged. Examples of terminal insertions include a TCR molecule with an N-terminal methionyl residue or the TCR molecule fused to an epitope tag. Other insertional variants of the TCR molecule include the fusion to the N- or C-terminus of the TCR of an enzyme or a polypeptide which increases the half-life of the TCR in the blood circulation.

The TCR, or antigen-binding portion thereof, may be linked to a payload or agent. Thus, there is provided herein an immunoconjugate comprising a TCR as disclosed herein, or antigen-binding portion thereof, linked to an agent.

The TCR may be linked to a therapeutic agent. For instance, the TCR, or antigen-binding portion thereof, may be linked to a substance that induces changes in cancer cells, such as a cytotoxic or radioactive agent. The TCR, or antigen-binding portion thereof, may be linked to a detectable agent or label, such as a radioactive or fluorescent agent.

Examples of suitable agents include cytotoxins, radioisotopes, chemotherapeutic agents, immunomodulatory agents, anti-angiogenic agents, antiproliferative agents, pro-apoptotic agents, and cytostatic and cytolytic enzymes (for example RNAses). Further agents include a nucleic acid, such as a gene encoding an immunomodulatory agent, an anti-angiogenic agent, an anti-proliferative agent, or a pro-apoptotic agent. These drug descriptors are not mutually exclusive, and thus an agent may be described using one or more of the above terms. Representative cytotoxins include antibiotics, inhibitors of tubulin polymerization, alkylating agents that bind to and disrupt DNA, and agents that disrupt protein synthesis or the function of essential cellular proteins such as protein kinases, phosphatases, topoisomerases, enzymes, and cyclins.

The linkage may be any suitable for linking the agent or payload to the TCR, or antigen-binding portion. For instance, the linkage may comprise a covalent or non-covalent bond. The linkage may be via a bi-functional linker. The linkage may be via two moieties capable of forming a non-covalent interaction, such as streptavidin and biotin.

In another aspect, there is provided at least one nucleic acid encoding a TCR, or antigen-binding portion thereof, as disclosed herein.

A single nucleic acid may encode both a TCRa chain and a TCRb chain. For instance, a TCRa chain comprising a TCRa variable region, or antigen-binding portion thereof, and a TCRb chain comprising a TCRb variable region, or antigen-binding portion thereof. The TCRa chain and TCRb chain may be encoded as a single polypeptide, and hence the nucleic acid may encode a single chain TCR. Alternatively, a single nucleic acid may encode a TCRa chain and a separate TCRb chain. For instance, under the control of separate promoters. In other examples, the TCRa chain and TCRb chain may be encoded by the same nucleic acid and under the control of the same promoter, but separated by a region that can induce ribosomal skipping or by a self-cleaving peptide sequence. Examples of such features include P2A, an internal ribosome entry site (IRES), or an intein.

Alternatively, a first nucleic acid chain may encode the TCRa chain and a second nucleic acid may encode the TCRb chain.

Thus, the nucleic acid molecule of the present disclosure may encode (a) the TCRa variable region amino acid sequence; (b) the TCRb variable region amino acid sequence; or (c) both the TCRa and the TCRb variable region amino acid sequence of a TCR, or antigen-binding portion, as disclosed herein. The nucleic acid molecule as defined herein may be isolated.

In another aspect, there is provided a vector comprising a nucleic acid as disclosed herein. The vector may be an expression vector or may be a vector designed for delivery to a subject, such as a human patient.

In another aspect, there is provided a cell comprising at least one nucleic acid as disclosed herein, or the vector as disclosed herein.

The cell may be a recombinant cell. The cell may be a cell suitable for commercial production of recombinant polypeptides and proteins. In some examples, the cell is mammal, bacteria, yeast, or plant derived. For instance, the cells may be CHO cells, NSO cells, HEK cells, E. coli cells, Pichia pastoris cells, or other suitable cells.

In another aspect, there is provided a method of producing a TCR or an antigen-binding portion thereof as disclosed herein, comprising culturing a cell disclosed herein under conditions that result in expression and/or production of the TCR or the antigen-binding portion thereof, and isolating the TCR or the antigen-binding portion thereof from the cell or culture.

In another aspect, there is provided a cell comprising a TCR or antigen-binding portion thereof as disclosed herein, at least one nucleic acid as disclosed herein, or a vector as disclosed herein.

The cell may be a mammalian or human cell. The cell may be an immune cell. The cell may be an immune cell suitable for use in immunotherapy. The immune cell may be a T cell, such as an alpha-beta T cell or a gamma-delta T cell. The alpha-beta T cell may be a CD4+ T cell or CD8+ T cell. The alpha-beta T cell may be a naïve cell, an effector cell, or a memory cell. The cell may be an invariant NK T cell. The cell may be an NK cell.

The immune cell may be a primary immune cell, such as an immune cell derived from PBMCs or a tumour sample. The immune cell may be a cell line, such as a T cell line or an NK cell line. The immune cell may have been differentiated in vitro, for instance from a stem cell. The stem cell may be an induced pluripotent stem cell (iPSC), a haematopoietic stem cell, or a mesenchymal stem cell (MSC). In a particular embodiment, the cell is a human NK cell that has been differentiated from iPSCs.

The cell may be in vitro and may be a T cell or an NK cell. The cell may be an in vitro population of T cells or an in vitro population of NK cells suitable for adoptive transfer therapy. The cell may be an in vitro population of human NK cells.

The TCR, or antigen-binding portion thereof, as disclosed herein may be provided in a lyophilised form for reconstitution prior to administration. For example, lyophilised proteins may be re-constituted in sterile water and mixed with saline prior to administration to an individual. A nucleic acid as disclosed herein or a vector as disclosed herein may be provided in a lyophilised form. A cell as disclosed herein may be provided in a stable form for storage, for instance, the cells may be frozen in the presence of a cryoprotectant. In other embodiments, all of these products may be provided in a form suitable for administration.

Thus, in another aspect, there is a provided a pharmaceutical composition comprising a TCR or antigen-binding portion as disclosed herein, at least one nucleic acid as disclosed herein, a vector as disclosed herein, or a cell as disclosed herein.

The pharmaceutical composition may comprise a pharmaceutically acceptable excipient, carrier, stabiliser, or diluent. A pharmaceutically acceptable excipient may be a compound or a combination of compounds entering into a pharmaceutical composition which does not provoke secondary reactions and which allows, for example, facilitation of administration, an increase in lifespan and/or efficacy in the body or an increase in solubility in solution. These pharmaceutically acceptable vehicles are well known and will be adapted by the person skilled in the art as a function of the mode of administration.

The TCRs and antigen-binding portions disclosed herein may be used to recognise MAGE-A4 expressing cells. Several types of cancer express MAGE-A4, hence the TCRs and portions may be used to recognise cancerous cells. In non-limiting examples, the TCRs and/or portions may be expressed by immune cells, thus endowing the immune cells with the capacity to recognise cancerous cells. In other examples, the TCRs or portions may be molecules that are independent of cells, and hence may direct payloads (such as cytotoxic agents) to tumour cells. In yet other examples, the TCRs may be part of bifunctional molecules and hence allow for tumour cell recognition by a molecule also capable of another function, such as T cell recruitment. The TCRs and portions need not be administered directly and may be expressed within the subject. Hence, the nucleic acids disclosed herein and the vectors disclosed herein may also be administered to patients.

Thus, in another aspect, there is provided a TCR or antigen-binding portion as disclosed herein, at least one nucleic acid as disclosed herein, a vector as disclosed herein, a cell as disclosed herein, or a pharmaceutical composition of as disclosed herein for use as a medicament.

In a particular embodiment, there is provided a human immune cell, such as a T cell or an NK cell, comprising a TCR, or antigen-binding portion thereof, for use as a medicament.

There is provided a method for enhancing an immune response in a subject, comprising administering to a subject in need thereof an effective amount of a TCR or antigen-binding portion as disclosed herein, at least one nucleic acid as disclosed herein, a vector as disclosed herein, a cell as disclosed herein, or a pharmaceutical composition as disclosed herein. The TCR, as administered or expressed, may enhance the immune response of the cells that expresses it and/or the cell expressing the TCR may enhance the response of cells in the same microenvironment, e.g. by the release of cytokines.

There is provided the use of a TCR or antigen-binding portion as disclosed herein, at least one nucleic acid as disclosed herein, a vector as disclosed herein, a cell as disclosed herein, or a pharmaceutical composition as disclosed herein, in the manufacture of a medicament.

The medicaments disclosed herein may be used in a method of treatment of the human or animal body, including prophylactic or preventative treatment (e.g. treatment before the onset of a condition in an individual to reduce the risk of the condition occurring in the individual; delay its onset; or reduce its severity after onset).

The terms “prophylactic”, “preventative”, or “preventing” and the like refer to reducing the probability of developing a disorder or condition in a subject, who does not have, but is at risk of or susceptible to developing a disorder or condition. Prevention and the like do not mean preventing a subject from ever getting the specific disease or disorder. Prevention may require the administration of multiple doses. Prevention can include the prevention of a recurrence of a disease in a subject for whom all disease symptoms were eliminated, or prevention of recurrence in a relapsing-remitting disease.

The method of treatment may comprise administering the medicament to an individual in need thereof.

The medicament may be for use in a mammalian subject. The medicament may be for use in a mouse, rat, rabbit, dog, cat, horse, or pig. Preferably, the subject is a human. The subject may have been determined to be HLA-A*0201 positive.

It is known in the art that TCRs specific for peptides presented by certain HLA subtypes may also recognise the target peptide presented in the context of other HLA subtypes, for instance related or similar subtypes. Thus, the subject may be determined to be positive for a HLA subtype to which a TCR, or antigen-binding portion thereof, of the present disclosure can bind.

Administration is normally in a “therapeutically effective amount”, this being sufficient to show benefit to a patient. Such benefit may be at least amelioration of at least one symptom. The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners. Prescription of treatment, e.g. decisions on dosage etc., is within the responsibility of medical professionals and may depend on the severity of the symptoms and/or progression of a disease being treated. A therapeutically effective amount or suitable dose of a medicament may be determined by comparing in vitro activity and in vivo activity in an animal model. Methods for extrapolation of effective dosages in mice and other test animals to humans are known. The precise dose will depend upon a number of factors, including whether the medicament is for prevention or for treatment, the size and location of the area to be treated, the precise nature of the medicament and the nature of any detectable label or other molecule attached to the antibody.

Administration may be via any suitable method, such as intra-venous administration, e.g. by injection. Administration may be of a single dose or may be repeated periodically.

The medicaments of the present disclosure may be administered as monotherapies or combination therapies with other agents. The administration of combination therapies may be either simultaneously or sequentially.

In another aspect, there is provided a TCR or antigen-binding portion as disclosed herein, at least one nucleic acid as disclosed herein, a vector as disclosed herein, a cell as disclosed herein, or a pharmaceutical composition as disclosed herein for use in a method for treating, preventing, or ameliorating cancer.

For instance, there is provided a method for treating, preventing, or ameliorating cancer in a subject, the method comprising administering to a subject in need thereof an effective amount of a TCR or antigen-binding portion as disclosed herein, at least one nucleic acid as disclosed herein, a vector as disclosed herein, a cell as disclosed herein, or a pharmaceutical composition as disclosed herein.

There is provided the use of a TCR or antigen-binding portion as disclosed herein, at least one nucleic acid as disclosed herein, a vector as disclosed herein, a cell as disclosed herein, or a pharmaceutical composition as disclosed herein, in the manufacture of a medicament for the treatment of cancer.

In a particular embodiment, there is provided a human immune cell, such as a T cell or an NK cell, comprising a TCR, or antigen-binding portion thereof, as disclosed herein for use in a method for treating, preventing, or ameliorating cancer.

The cancer may be a solid cancer. The cancer may comprise cells expressing MAGE-A4. Some cancerous cells may display a peptide according to SEQ ID NO: 122. The subject may have precancerous cells expressing MAGE-A4.

In one embodiment, the cancer is MAGE-A4 positive and is lung cancer, head and neck cancer, liver cancer, skin cancer, renal cell cancer, brain cancer, gastric cancer, colorectal cancer, hepatocellular cancer, pancreatic cancer, prostate cancer, leukemia, breast cancer, Merkel cell carcinoma, urinary bladder cancer, uterine cancer, gallbladder and bile duct cancer, osteosarcoma, liposarcoma, a neuroblastoma, a myeloma, a melanoma, a metastatic melanoma, a synovial sarcoma, an esophageal cancer, an esophageal squamous cell carcinoma, an ovarian cancer, an ovarian epithelial cancer, a prostate cancer, a breast cancer, an astrocytic tumor, a glioblastoma multiforme, an anaplastic astrocytoma, a fallopian tube cancer, primary peritoneal cavity cancer, advanced solid tumors, soft tissue sarcoma, a sarcoma, a myelodysplastic syndrome, an acute myeloid leukemia, a Hodgkin lymphoma, a non-Hodgkin lymphoma, a Hodgkin disease, a multiple myeloma, a metastatic solid tumors, a stomach cancer, a gastric cancer, a rhabdomyosarcoma, a myxoid round cell liposarcoma, or a recurrent non-small cell lung cancer.

In examples, the cancer is MAGE-A4 positive and is lung cancer, liver cancer, head and neck cancer, skin cancer, gastric cancer, colorectal cancer, hepatocellular cancer, pancreatic cancer, breast cancer, melanoma, ovarian cancer, urinary bladder cancer, uterine cancer, gallbladder and bile duct cancer, and esophageal cancer.

In some examples, there is provided herein a method of treating, preventing, or ameliorating lung cancer, esophagus carcinoma, head and neck cancer, ovarian cancer, melanoma.

The lung cancer may be non-small cell lung cancer (NSCLC), such as non-small cell lung cancer adenocarcinoma, squamous cell non-small cell lung cancer (SNSCLC), or small cell lung cancer (SCLC). The skin cancer may be melanoma. The head and neck cancer may be head and neck squamous cell carcinoma (HNSCC). The liver cancer may be hepatocellular cancer (HCC). The esophageal cancer may be gastroesophageal junction cancer.

In examples, the cancer is MAGE-A4 positive and is an ovarian cancer, a melanoma, a non-small cell lung carcinoma, a hepatocellular carcinoma, a colorectal carcinoma, an esophageal squamous cell carcinoma, an esophageal adenocarcinoma, a stomach cancer, a bladder cancer, a head and neck cancer, a gastric cancer, a synovial sarcoma, or a myxoid round cell liposarcoma.

In a preferred embodiment, the cancer is NSCLC, ovarian, gastroesophageal, head and neck synovial sarcoma, or urothelial.

Sequence comparisons can be conducted with the aid of readily available sequence comparison programs. These publicly and commercially available computer programs can calculate sequence identity between two or more sequences.

The skilled technician will appreciate how to calculate the percentage identity between two nucleic sequences or amino acid sequences. In order to calculate the percentage identity, an alignment of the two sequences must first be prepared, followed by calculation of the sequence identity value. The percentage identity for two sequences may take different values depending on: (i) the method used to align the sequences, for example, the Needleman-Wunsch algorithm (e.g. as applied by Needle (EMBOSS) or Stretcher (EMBOSS), the Smith-Waterman algorithm (e.g. as applied by Water (EMBOSS)), or the LALIGN application (e.g. as applied by Matcher (EMBOSS); and (ii) the parameters used by the alignment method, for example, local versus global alignment, the matrix used, and the parameters applied to gaps. In a particular embodiment, the sequence identities disclosed herein may be calculated based on a global alignment of the relevant feature. For instance, the comparison of a complete length of a TCR variable region within a polypeptide to the complete reference sequence for a TCR variable region recited herein. In some embodiments, there is disclosed an antigen-binding portion of a recited TCR sequence and, in an example, the comparison may be over the complete length of the antigen-binding portion to the same length of the respective fragment of the sequence recited herein.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length-dependent. Therefore, the shorter a pair of sequences is, the higher the sequence identity one may expect to occur by chance.

A calculation of percentage identities between two nucleic acid sequences may then be calculated from such an alignment as (N/T)*100, where N is the number of positions at which the sequences share an identical residue, and T is the total number of positions compared including gaps but excluding overhangs.

The sequence alignment may be a pairwise sequence alignment. Suitable services include Needle (EMBOSS), Stretcher (EMBOSS), Water (EMBOSS), Matcher (EMBOSS), LALIGN, or GeneWise. In an example, the identity between two amino acid sequences may be calculated using the service Needle (EMBOSS) set to the default parameters, e.g. matrix (BLOSUM62), gap open (10), gap extend (0.5), end gap penalty (false), end gap open (10), and end gap extend (0.5). In another example, the identity between two amino acid sequences may be calculated using the service Matcher (EMBOSS) set to the default parameters, e.g. matrix (BLOSUM62), gap open (14), gap extend (4), alternative matches (1). In an example, the identity between two nucleic acid sequences may be calculated using the service Needle (EMBOSS) set to the default parameters, e.g. matrix (DNAfull), gap open (10), gap extend (0.5), end gap penalty (false), end gap open (10), and end gap extend (0.5). In another example, the identity between two nucleic acid sequences may be calculated using the service Matcher (EMBOSS) set to the default parameters, e.g. matrix (DNAfull), gap open (16), gap extend (4), alternative matches (1).

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made to the Examples, which are not intended to limit the invention in any way.

### EXAMPLES

### Example 1—Parent TCR

In order to produce new MAGE-A4 TCRs, a parental TCR was chosen for analysis. The parental TCR targets the peptide KVLEHVVRV (SEQ ID NO: 122) in the context of HLA-A*0201 and is disclosed in WO2021/236548 A1 (herein incorporated by reference). The sequences of the parent TCR are recited in the table below. This TCR is, in places, referred to as “wild type” herein.

TABLE 5

| | | SEQ ID |
|---|---|---|
| Name of sequence | Amino acid sequence | NO |
| TCR sequence; | MKREREMLLITSMLVLWMQLSQVNGQQVMQIPQYQHVQEGEDFTTYCNSSTTLSNIQWY | 1 |
| Alpha chain | KQRPGGHPVFLIQLVKSGEVKKQKRLTFQFGEAKKNSSLHITATQTTDVGTYFCAVGSM | |
| (TRAV25*01F) | DSNYQLIWGAGTKLIIKPDIQNPDPAVYQLRDSKSSDKSVCLFTDFDSQTNVSQSKDSD | |
| | VYITDKTVLDMRSMDFKSNSAVAWSNKSDFACANAFNNSIIPEDTFFPSPESSCDVKLV | |
| | EKSFETDTNLNFQNLSVIGFRILLLKVAGFNLLMTLRLWSS | |
| Signal Peptide | MKREREMLLITSMLVLWMQLSQVN | 2 |
| alpha-V region | GQQVMQIPQYQHVQEGEDETTYCNSSTTLSNIQWYKQRPGGHPVFLIQLVKSGEVKKQK | 3 |
| | RLTFQFGEAKKNSSLHITATQTTDVGTYFCAVGSMDSNYQLIWGAGTKLIIKP | |
| alpha-C region | DIQNPDPAVYQLRDSKSSDKSVCLFTDFDSQTNVSQSKDSDVYITDKTVLDMRSMDFKS | 4 |
| | NSAVAWSNKSDFACANAFNNSIIPEDTFFPSPESSCDVKLVEKSFETDTNLNFQNLSVI | |
| | GFRILLLKVAGFNLLMTLRLWSS | |
| CDR1-alpha | TTLSN | 5 |
| CDR2-alpha | LVKSGEV | 6 |
| CDR3-alpha | AVGSMDSNYQLI | 7 |
| TCR sequence; | MSLGLLCCGAFSLLWAGPVNAGVTQTPKFRVLKTGQSMTLLCAQDMNHEYMYWYRQDPG | 8 |
| Beta chain | MGLRLIHYSVGEGTTAKGEVPDGYNVSRLKKQNFLLGLESAAPSQTSVYFCASSQERTW | |
| (TRBV06-2*01F, | PYNEQFFGPGTRLTVLEDLKNVFPPEVAVFEPSEAEISHTQKATLVCLATGFFPDHVEL | |
| or | SWWVNGKEVHSGVSTDPQPLKEQPALNDSRYCLSSRLRVSATFWQNPRNHFRCQVQFYG | |
| TRBV06-3*01F) | LSENDEWTQDRAKPVTQIVSAEAWGRADCGFTSVSYQQGVLSATILYEILLGKATLYAV | |
| | LVSALVLMAMVKRKDF | |
| Signal Peptide | MSLGLLCCGAFSLLWAGPV | 9 |
| beta-V region | NAGVTQTPKFRVLKTGQSMTLLCAQDMNHEYMYWYRQDPGMGLRLIHYSVGEGTTAKGE | 10 |
| | VPDGYNVSRLKKQNFLLGLESAAPSQTSVYFCASSQERTWPYNEQFFGPGTRLTVL | |
| beta-C region | EDLKNVEPPEVAVFEPSEAEISHTQKATLVCLATGFFPDHVELSWWVNGKEVHSGVSTD | 11 |
| | PQPLKEQPALNDSRYCLSSRLRVSATFWQNPRNHFRCQVQFYGLSENDEWTQDRAKPVT | |
| | QIVSAEAWGRADCGFTSVSYQQGVLSATILYEILLGKATLYAVLVSALVLMAMVKRKDF | |
| CDR1-beta | MNHEY | 12 |
| CDR2-beta | SVGEGT | 13 |
| CDR3-beta | ASSQERTWPYNEQF | 14 |

### Example 2—Structural Biological Analysis of Parent TCR

Structural biological analysis was performed for the parent TCR when bound to peptide-HLA.

Crystal diffraction data were collected. The data were scaled with STARANISO with to a resolution of 2.39 Å. The structure was then solved by molecular replacement using structural models from the protein data bank (PDB). Molecular replacement searching for a single copy of pHLA followed by a single copy TCR was successful and the mode of binding and the protein-protein interface could be clearly defined.

The crystal structure revealed that the majority of interactions are made through the TCR beta chain and, in particular, the most interactions are made by CDR3b.

In particular, the underlined residues in the sequence below represent residues of CDR3b that interact with the HLA molecule. The bold residues represent those that interact with the peptide. CASSQERTWPYNEOFF (SEQ ID NO: 57)

Of the contribution that is made by the alpha chain, CDR3a is responsible for most of the interactions.

In particular, the underlined residues in the sequence below represent residues that interact with the HLA molecule. The bold residues represent those that interact with the peptide.

(SEQ ID NO: 26)
CAVGSMDSNYQLIW

### Example 3—Library Generation—In Silico Modelling

We used an alanine scanning screening method to map key amino acid residues in the TCR-pMHC binding interface.

Then a first TCR library, comprising mutations of the parent TCR (also known as the WT TCR), was prepared using artificial intelligence. This involved use of a machine learning platform that is trained to find patterns in TCR repertoires and apply these patterns to a particular TCR. Part of the in silico modelling was expert-guided.

The first arm of the modelling was performed based on the sequence alone and the alanine scanning data set. Four 3D structural models were made of the TCR docked to a peptide-HLA complex. Variants were then produced both by expert-guided mutagenesis and in silico generation. The effects of the mutations on the 3D model were then assessed in silico.

The variants were tested against the modelled structures to search for predicted increased affinity.

Changes such as alterations in AG, total buried surface area, pHLA solvent accessibility surface area (SASA) ratio, the number of hydrogen bonds which interact with peptide-HLA, and the number of Van der Waals interactions to peptide-HLA were assessed. Candidate TCRs were then selected for further testing.

### Example 4—Binding Analysis of TCR Variants

A subset of the TCRs arising from the in silico modelling (Example 3) went into binding analysis, as set out below

### Molecular Cloning for Protein Expression

The Novagen pET21(+) expression plasmid was used for bacterial expression using E. coli (BL21 DE3) as a host organism. Due to the nature of the pHLA and TCR domains, they are expressed as insoluble inclusion bodies (IB). The total protein expressed can be more than 50% of the total cell protein. IB preparation, solubilisation and refolding of TCRs and pHLAs are well established methods (e.g. Willcoj et al., Protein Science (1999), 8:2418-2423, https://doi.org/10.1110/ps.8.11.2418; Boulter et al., Protein Engineering vol. 16 no. 9 pp. 707-711, 2003, https://doi.org/10.1093/protein/gzg087; or Garboczi et al., PNAS, Vol. 89, pp. 3429-3433 April 1992, https://doi.org/10.1073/pnas.89.8.3429) and often yield highly pure, homogenous protein preparations (free of N-linked glycosylation).

### TCR Construct Design

Clones were made in the bacterial expression vector pET21(+). The alpha and beta chains were cloned into the vector using the NdeI and NotI restriction sites making use of the ATG start codon in the NdeI site to initiate transcription ensuring no extra amino acids were expressed at the N-terminus of the protein chain. TCR sequences were truncated to exclude the transmembrane region and modified through incorporation of an unnatural alpha-beta disulphide bond between the constant domains (Boulter et al., 2003) and substitution of a free cysteine to produce soluble and stabilised proteins.

The TCR alpha constant domain (UNIPROT P01848) was modified at position 47; a threonine replaced with a cysteine and the domain terminates at position 85 (as in SEQ ID NO: 67).

(SEQ ID NO: 67)
IQNPDPAVYQLRDSKSSDKSVCLFTDFDSQTNVSQSKDSDVYITDKCVLDMRSMDFKSNSAVAWSNKSDFACANAF
NNSIIPEDT.

The TCR beta constant domain (UNIPROT A0A5B9) was modified at position 9; a lysine replaced by a glutamic acid, position 56; a serine replaced by a cysteine, position 74; a cysteine replaced by an alanine, position 88; an asparagine replaced by an aspartic acid and the domain terminates at position 129 (as in SEQ ID NO: 68).

(SEQ ID NO: 68)
DLKNVFPPEVAVFEPSEAEISHTQKATLVCLATGFYPDHVELSWWVNGKEVHSGVCTDPQPLKEQPALNDSRYALS
SRLRVSATFWQDPRNHFRCQVQFYGLSENDEWTQDRAKPVTQIVSAEAWGRAD.

### Human Leukocyte Antigen (HLA) and Beta 2 Microglobulin (B2M) Construct Design

Constructs used for the production of pHLA protein complex (biotinylated monomers) were produced in the pET21a vector using the start codon of the NdeI restriction site to initiate transcription. The HLA A201 construct (pET21|HLA_A2_25-300_Avi) codes for HLA A201 amino acids 25-300 (regions alpha 1, 2 and 3) the construct terminates the transmembrane region and leads directly into a biotinylation sequence (AviTag: GLNDIFEAQKIEWHE (SEQ ID NO: 69). The beta 2 microglobulin construct (pET21|B2M_21-119), matches the naturally secreted protein: amino acids 21-119.

(pET21|HLA_A2_25-300_Avi-SEQ ID NO: 70)
MGSHSMRYFFTSVSRPGRGEPRFIAVGYVDDTQFVREDSDAASQRMEPRAPWIEQEGPEYWDGETRKVKAHSQTHR
VDLGTLRGYYNQSEAGSHTVQRMYGCDVGSDWRFLRGYHQYAYDGKDYIALKEDLRSWTAADMAAQTTKHKWEAAH
VAEQLRAYLEGTCVEWLRRYLENGKETLQRTDAPKTHMTHHAVSDHEATLRCWALSFYPAEITLTWQRDGEDQTQD
TELVETRPAGDGTFQKWAAVVVPSGQEQRYTCHVQHEGLPKPLTLRWEPGLNDIFEAQKIEWHE.
(pET21|B2M_21-119-SEQ ID NO: 123)
MIQRTPKIQVYSRHPAENGKSNFLNCYVSGFHPSDIEVDLLKNGERIEKVEHSDLSFSKDWSFYLLYYTEFTPTEK
DEYACRVNHVTLSQPKIVKWDRDM.

### MAGE-A4 286-294 and Mimetic Peptides

The modified TCR (modified from WT as discussed above) and affinity enhanced TCR variants were used in surface plasmon resonance (SPR) experiments to estimate the binding affinity to the cognate MAGE-A4 (286-294) peptide-HLA complex. A set of 15 pHLA complexes harbouring sequence related mimetic peptides were also assessed for potential cross reactivity (below).
KVLEHVVRV (MAGE-A4_286-294 (Melanoma-associated antigen 4, Uniprot P43358. MAGA4_HUMAN—SEQ ID NO: 122
FLLETVVRV (RABGAPIL_408-416 (RAB GTPase Activating Protein 1 Like, Uniprot: Q5R372⋅RBG1L_HUMAN—SEQ ID NO: 124)
ILDEHVORV (AXIN1_470-478 (Axin-1, Uniprot: 015169⋅AXIN1_HUMAN)—SEQ ID NO: 125)
KILEDVVGV (TPX₂_465-473 (Targeting protein for Xklp2, Uniprot: Q9ULW0⋅TPX₂_HUMAN—SEQ ID NO: 126)
KLLDLQVRV (SYNE3_563-571 (Nesprin-3, Uniprot: Q6ZMZ3⋅SYNE3_HUMAN—SEQ ID NO: 127)
KVLEILHRV (HERC4_595-603 (Probable E3 ubiquitin-protein ligase HERC4, Uniprot: Q5GLZ8⋅HERC4_HUMAN—SEQ ID NO: 128)
KVLERVNAV (PSME2_145-153 (Proteasome activator complex subunit 2, Uniprot: Q9UL46⋅PSME2_HUMAN—SEQ ID NO: 129)
KVLETLVTV (HEATR5A_1925-1933 (HEAT repeat-containing protein 5A, Uniprot: Q86XA9⋅HTR5A_HUMAN—SEQ ID NO: 130)
KVLGIVVGV (CNOT1_1965-1973 (CCR4-NOT transcription complex subunit 1, Uniprot: A5YKK6⋅CNOT1_HUMAN—SEQ ID NO: 131)
KVLNKVITV (KIAA1033_85-93 (WASC4_HUMAN WASH complex subunit 4, Uniprot: Q2M389⋅WASC4 HUMAN—SEQ ID NO: 132)
KVTEMLVNV (PPP3CB_369-377 (Serine/threonine-protein phosphatase 2B catalytic subunit beta isoform, Uniprot: P16298⋅PP2BB_HUMAN—SEQ ID NO: 133)
KVLETTVEI (VPS35_371-379 (Vacuolar protein sorting-associated protein 35, Uniprot: Q96QK1⋅VPS35_HUMAN—SEQ ID NO: 134)
KVLEMETTV (RNF40_271-279 (E3 ubiquitin-protein ligase BREIB, Uniprot: O75150⋅BRE1B_HUMAN—SEQ ID NO: 135)
KVMEHVYEK (WDR48_595-603 (WD repeat-containing protein 48, Uniprot: Q8TAF3⋅WDR48_HUMAN—SEQ ID NO: 136)
KVQEQVHKV (NSD1_1300-1308 (Histone-lysine N-methyltransferase, H3 lysine-36 specific, Uniprot: Q96L73⋅NSD1_HUMAN—SEQ ID NO: 137)

### Protein Expression, Refolding, and Purification of Soluble Biotinylated Class I HLA-A*02 Protein Complexes

HLA and B2M expression, IB isolation, protein refolding in the presence of peptide of interest, biotinylation and purification were performed using established methods known to those in the art (Garboczi et al. (1992) or O'Callaghan et al., Analytical Biochemistry, Volume 266, Issue 1, 1 Jan. 1999, Pages 9-1, https://doi.org/10.1006/abio.1998.2930).

Briefly, HLA and B2M proteins were expressed and IBs isolated as described here for TCR. Solubilised and reduced HLA, B2M and peptide (at a molar ratio of 1:2:4) were refolded by rapid dilution into a refold buffer (0.4 M L-arginine-HCl, 100 mM Tris pH 8.0, 2 mM EDTA, 6.6 mM cysteamine hydrochloride and 3.7 mM cystamine dihydrochloride). Refolded material was dialysed into 10 mM Tris-HCl pH 8.1 and purified by anion exchange, biotinylated in-vitro and purified by size exclusion chromatography.

### Protein Expression, Refolding, and Purification of Soluble TCRs

DNA sequences encoding the variable and constant regions of TCR alpha and TCR beta chains were cloned into the pET21(+) expression plasmid between the NdeI and NotI restriction sites using standard methods. The TCR alpha and TCR beta plasmids were transformed separately into the E. coli strain Rosetta 2(DE3)pLysS. Individual colonies were used to inoculate LB (+ampicillin; 100 μg/ml) medium and grown at 37° C. until an OD₆₀₀ of 0.5 was reached. The culture was then used to inoculate auto induction medium (AIM)—Super Broth (ForMedium) supplemented with ampicillin at 100 μg/ml), grown at 37° C. for 5 hours and 30° C. for a further 16 hours. Cells were harvested by centrifugation. Cell pellets were lysed using BugBuster® Protein Extraction Reagent (Merck Millipore) following the manufactures instructions and inclusion bodies were recovered by centrifugation. Inclusion bodies were washed three times using Triton wash buffer (50 mM Tris pH 8.1, 100 mM NaCl, 1% Triton-X100 and 10 mM EDTA) and once in resuspension buffer (50 mM Tris pH 8.1, 100 mM NaCl, and 10 mM EDTA) and finally resuspended in the same buffer. Inclusion body protein concentration was estimated by solubilising in solubilisation buffer (6M guanidine hydrochloride, 50 mM Tris pH 8.1, 100 mM NaCl and 10 mM EDTA) and measuring the OD₂₈₀ and calculated considering the extinction coefficient. Inclusion body purity was assessed by solubilising ~2 μg protein in 8M urea and SDS-PAGE under reducing conditions. Purity was estimated by densitometry (GelDoc, Bio-Rad). Inclusion bodies were stored at +4° C. or −80° C. for short or long storage, respectively.

TCR refolding, 45 mg alpha and 30 mg beta chain inclusion bodies were mixed and solubilised in 10 ml denaturation buffer (6M guanidine hydrochloride, 50 mM Tris pH 8.1, 100 mM NaCl, 10 mM EDTA and 20 mM DTT) and incubated for 10 min at 30° C. Refolding was achieved through rapid dilution into 1 L vigorously stirring refold buffer (100 mM Tris pH 8.1, 400 mM L-arginine-HCl, 4M urea, 2 mM EDTA, 6.5 mM cysteamine hydrochloride and 1.9 mM cystamine dihydrochloride) and incubated for 3 hours at 4° C. The protein solution was dialysed into 20 L water for 16 hours at 4° C. The protein solution was further dialysed, into 20 L 10 mM Tris pH 8.1 overnight, this step was repeated. The protein solution was then filtered through a 0.45 μm cellulose membrane.

TCRs were purified by anion exchange followed by size exclusion chromatography. Protein solution was passed over a 5 ml Q Sepharose (Cytiva) column and eluted with a continuous gradient of 0-500 mM NaCl in 10 mM Tris pH8.1 over 25 column volumes using a FPLC machine (Cytiva). Protein fractionation was assessed by SDS-PAGE and TCR heterodimer fractions were pooled and concentrated. Concentrated TCR protein was further purified by size exclusion chromatography using a Superdex 200 Increase column into PBS (10 mM phosphate buffer, 2.7 mM KCl and 137 mM NaCl. The concentration of pooled peak TCR fractions were estimated by measuring the OD₂₈₀ and calculated considering the extinction coefficient.

### Binding Analysis

Affinity analysis for soluble TCRs binding to pHLA complexes was carried out by surface plasmon resonance (SPR) using a BIAcore T200 instrument. All measurements were carried out at 25° C.

### SPR Method

Biotinylated peptide HLA monomers were immobilised on a sensor chip preloaded with streptavidin (Sensor Chip SA). Equilibrium binding constants were determined by flowing serial dilutions of TCRs at 10 μl/minute for 60 seconds over flow cells loaded with between 50 and 600 response units of peptide-HLA complex. Specific responses were determined by subtracting the non-specific response of a flow cell immobilised with only streptavidin from the response of a flow cell loaded with relevant peptide-HLA.

The K_{d} value was determined by nonlinear curve fitting using Prism software and the binding isotherm equation equivalent to the Langmuir isotherm for the absorption of a gas onto a surface.

$$Y = Bmax * X / ( {K}_{d} + X )$$

K_{d} is the equilibrium dissociation constant, in the same units as X (Molar). It is the concentration needed to achieve a half-maximum binding at equilibrium. Bmax is the maximum specific binding in the same units as Y (RU). Y is the equilibrium binding in RU at injected TCR concentration X.

For the highest TCR affinity interaction there was sufficient curvature in the injection and dissociation phases of the sensorgrams that it was possible to fit the kinetics parameters kₒₙ and k_{off}, this was performed in the BIAevaluation software. K_{d} was calculated from k_{off}/kₒₙ and the dissociation half-life was calculated: t1/2=0.693/k_{off}.

### Results

The experiments set out above identified a particular variant CDR3b (CASSQERTQPYNEQFF (SEQ ID NO: 59).

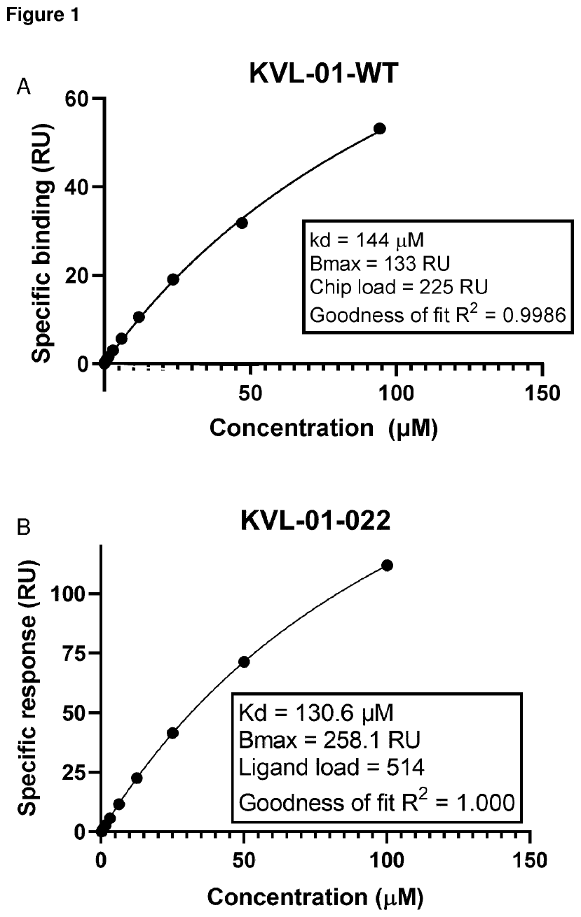

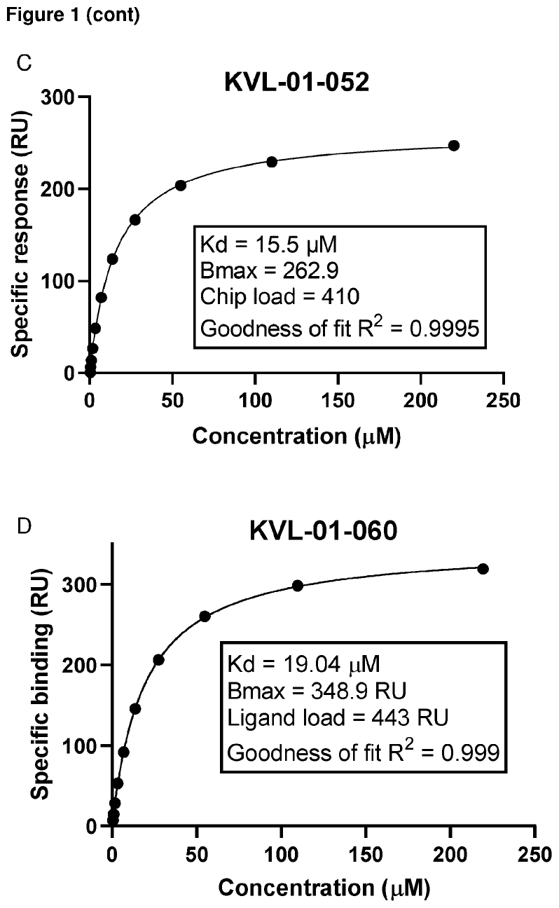

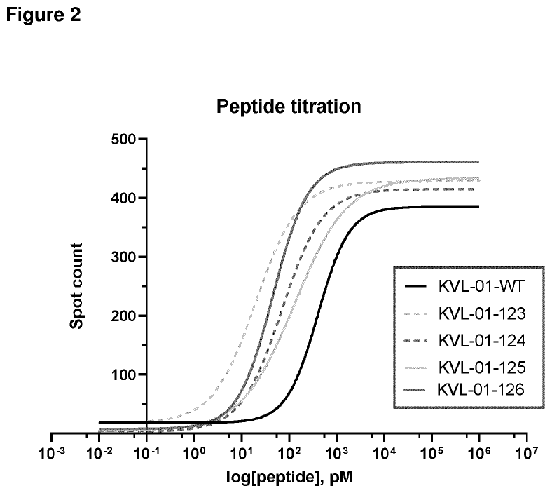

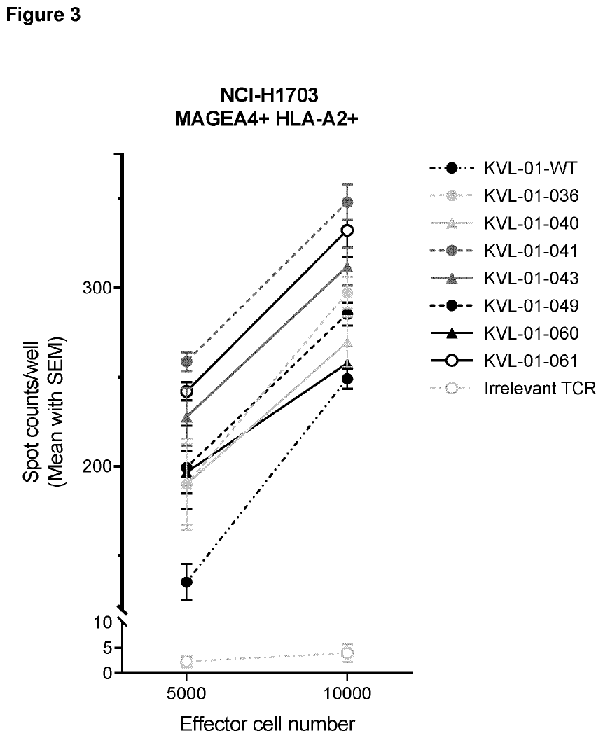

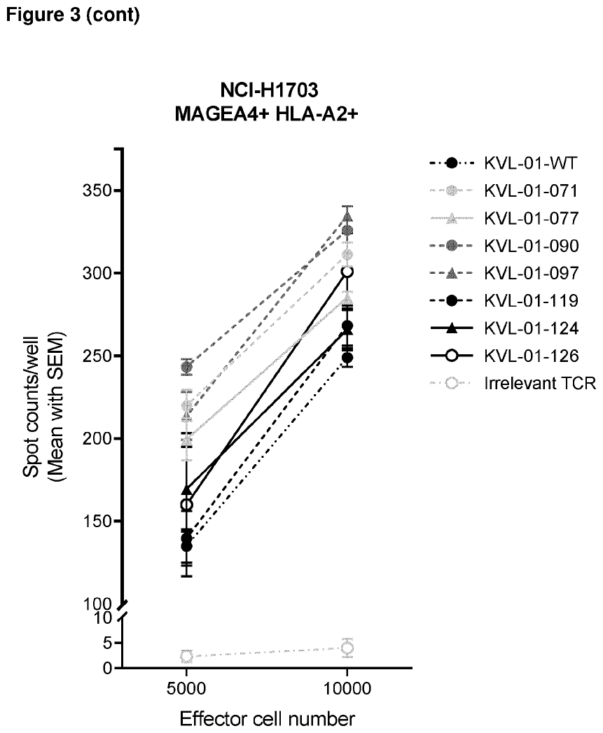

This variant was tested by surface plasmon resonance (SPR). It was found that the CDR3b W-Q mutation led to an 8.3 fold increase in K_{d} compared to the parental TCR (to 19 μM). This is illustrated in FIG. 1(D). Alternative mutations were tested as set out in Example 6.

Another variant that was identified was CAVGSNQSNYQLIW (SEQ ID NO: 51). The NQx motif is found in several CDR3a sequences disclosed herein (e.g. SEQ ID NOs: 35 to 44-experimentally tested as set out in Example 6). This motif was predicted to alter the potential energy on binding from −1765.34 kcal/mol to −2018.19 kcal/mol.

The M94N, D95Q, S96 and W99 mutations were tested in combination (see Example 6-in particular KVL-01-073 to KVL-01-77) and found to be functional.

### Example 5—Library Generation

### In Silico Prediction of Favourable TCR Framework and CDR Mutations

A set of TCR single-point mutations targeting the framework and CDRs of the TRA and TRB chains were predicted based on supervised structural assessment of custom built TCR and pHLA homology models and optimized trimeric TCR-pHLA in silico docking complexes. Initial TCR and pHLA models were built using LYRA (Klausen, M S et al. Nucleic Acids Res 43, W349-355, doi:10.1093/nar/gkv535 (2015)) and DockTope (Menegatti Rigo, M. et al. Scientific reports 5, 18413, doi:10.1038/srep18413 (2015)), respectively. The analysis involved assessment of TCR scaffold charge and folding liabilities, as well as improving suboptimal TCR-pHLA interaction regions rationalized by in silico mutagenesis evaluation based on alternatives offered through rotamer library scans. Only candidate mutations in accordance with wet lab interaction profiles (such as the alanine scanning data) were taken forward to subsequent wet lab confirmation as TCR reconstructed SKW3 cells, which gave rise to secondary TCR combination mutant versions validated in the same manner.

In particular, the alanine scanning data confirmed the importance of W99 in the beta chain. Replacement of the seventh residue of KVLEHVVRV (SEQ ID NO: 122) with alanine reduced recognition of the pHLA, as measured by ELISPOT. This is hypothesised to be due to an effect on the stabilisation of the TCRbeta^{CDR3-W99} lynchpin.

### CDR1 and CDR2 Library Design and Generation

A second TCR library, comprising mutations in the CDR1 and CDR2 of the parent TCR (also known as the WT TCR), was prepared.

To generate variants, different approaches were used: i) a focused unbiased approach, and ii) a focused informed approach, which is based on structural models and cross examination of known structures.

i) Involved a hard randomisation of CDR1a, CDR2a, and CDR1b. ii) Involved a focus on positions in proximity to key residues likely to be involved in peptide contact, HLA contact, or CDR stabilisation.

The quality of the CDR1 and CDR2 libraries were measured by sequencing to assess the quality of the insert, to check the frequency of the background, and to check the ratio between mixed libraries. The functionality of the library was tested by cellular expression of the TCRs, activation with 10 μM MAGE-A4 peptide, and flow cytometric analysis, as described in more detail in Example 4 and below.

### Generation of CDR1 and CDR2 Libraries

The designed TCR libraries were provided as sub-cloning compatible dsDNA. The CDR loop definitions and the library designs were based on IMGT and predicted 3D models. The libraries were subcloned into the pMSCVneo vector (Clontech) harbouring the target gene with murine TCR domains (Cohen, C. J. et al., Cancer research 66, 8878-8886, doi:10.1158/0008-5472.CAN-06-1450 (2006)). The resulting plasmid DNA for cell transfection was prepared from resulting E. coli material using standard methods.

Plasmid DNA from the libraries was transfected into GP2-293 packaging cells (ClonTech) along with pAmpho (ClonTech) packaging vector using Lipofectamine 2000 transfection reagent (Invitrogen). Retroviral supernatant was used to transduce target cells (SKW-3, Cell Line Services, Eppelheim, Germany). Transduced SKW-3 cells were stained with H57-PE (BD Pharmingen) and anti-CD3-A647 (BD Biosciences) mAbs to identify the TCR positive population. Double positive events were isolated on a SONY SH800 Flow Cytometer.

### Functional Selection of Cellularly Displayed TCR CDR1 and CDR2s

Briefly, T2 cells (ATCC) were pulsed with 10 nM KVL peptide (Genscript) and co-cultured with SKW-3 cells harbouring the library. After 16 hours co-culture, cells were stained with a-HLA-A2-FITC (BD Pharmingen), a-CD25-PE (Miltenyi Biotech) and a-CD69-APC (BD Biosciences) mAbs, before HLA-A2 negative and CD25 and CD69 double positive cells were sorted as single cells into growth medium with 20% FCS using either a Sony SH800 or BD FACSAria II cytometer. Expanded single cells were sequenced to reveal clonal identity. Briefly, 40000 cells were lysed using Cell Lysis Buffer (TaKaRa). RNA transcribing library candidates were amplified using One-Step RT-PCR kit (QIAGEN). Resulting PCR products were sequenced (GeneWiz) and standard sequence analysis tools were used to reveal clonal identity.

### Example 6—Screening of Variants

The TCR panels generated by the experiments of Examples 3 to 5 were then screened. The candidate TCRs tested and disclosed herein are listed in the table below.

TABLE 6

| TCR ID | Alpha mutations | Beta mutations |
|---|---|---|
| KVL-01-018 | L49W | |
| KVL-01-019 | V50P | |
| KVL-01-020 | K51L | |
| KVL-01-022 | G53P | |
| KVL-01-026 | M94W | |
| KVL-01-034 | L29T, M94R, I101V | |
| KVL-01-036 | M94R, I110V | M32L |
| KVL-01-039 | L29T, I101V, I110V | M32L |
| KVL-01-040 | M94R, I101V, I110V | M32L |
| KVL-01-041 | L29T, M94R, I110V | M32L |
| KVL-01-042 | L29T, M94R, I101V, | M32L |
| | I110V | |
| KVL-01-043 | M94R | |
| KVL-01-048 | L49W | W99Q |
| KVL-01-049 | V50P | W99Q |
| KVL-01-050 | K51L | W99Q |
| KVL-01-052 | G53P | W99Q |
| KVL-01-056 | M94W | W99Q |
| KVL-01-060 | | W99Q |
| KVL-01-061 | M94R | W99Q |
| KVL-01-068 | M94N, D95Q, S96R | |
| KVL-01-069 | M94N, D95Q, S96D | |
| KVL-01-070 | M94N, D95Q, S96E | |
| KVL-01-071 | M94N, D95Q, S96M | |
| KVL-01-073 | M94N, D95Q, S96R | W99Q |
| KVL-01-074 | M94N, D95Q, S96D | W99Q |
| KVL-01-075 | M94N, D95Q, S96E | W99Q |
| KVL-01-076 | M94N, D95Q, S96M | W99Q |
| KVL-01-077 | M94N, D95Q, S96V | W99Q |
| KVL-01-078 | L29T, V50E, M94R, I101V | |
| KVL-01-079 | L29T, V50G, K51Q, M94R, | |
| | I101V | |
| KVL-01-083 | L29T, V50E, M94R | |
| KVL-01-084 | L29T, V50G, K51Q, M94R | |
| KVL-01-088 | L29T, M94R | |
| KVL-01-089 | L29T | |
| KVL-01-090 | T27D, T28A, L29S, S30T, | |
| | N31E, V50E, M94R | |
| KVL-01-094 | T27S, T28R, L29S, S30G, | |
| | V50G, K51Q, M94R | |
| KVL-01-097 | T27S, T28R, L29S, S30G, | A56T, G40D |
| | M94R | |
| KVL-01-105 | V50G, K51Q, M94R | |
| KVL-01-108 | V50E, M94R | |
| KVL-01-110 | M94R, I101V | |
| KVL-01-111 | V50G, K51Q, M94R, I101V | |
| KVL-01-112 | V50E, M94R, I101V | |
| KVL-01-117 | M94R, D95Q | |
| KVL-01-119 | V50P, D95Q | |
| KVL-01-123 | M94R, D95Q, V50P | W99Q |
| KVL-01-124 | M94R, D95Q, V50P | |
| KVL-01-125 | V50P, D95Q | |
| KVL-01-126 | V50P, M94R | |
| KVL-01-128 | V50P, M94R | T54N |
| KVL-01-129 | M94R | W99A |
| KVL-01-WT | | |
| KVL-01-145 | M94V, D95N, S96T | |
| KVL-01-149 | V50E, M94V, D95N, S96T | |
| KVL-01-151 | M94V, D95N, S96T | G40D, A56T |
| KVL-01-153 | M94N, D95Q, S96V | G40D, A56T, W99Q |

Viral vectors were prepared for the transduction of peripheral blood mononuclear cells (PBMCs) with the candidate TCRs. All TCRs in the above table had adequate expression in the cells. These cells were then tested in an IFNγ ELISPOT assay to measure each TCR's activity against antigen presenting cells pulsed with the target peptide. The cells were also tested against various cancer cell lines, including NCI-H1703 and C33A, both of which express the target peptide in the correct HLA context.

TABLE 7

| | | | | Response | Response | |
|---|---|---|---|---|---|---|
| | | | 2\hat{ }-log | to NCI- | to | |
| | | | EC50 | H1703 | C33A | |
| | | | Fold | fold | fold | |
| | | | change | change | change | |
| | | 2\hat{ }-log | vs. | vs. | vs. | Kd |
| TCR ID | EC₅₀ | EC50 | WT | WT | WT | (uM) |
| KVL-01-014 | 3,310 | 0.09 | 1.4 | | | |
| KVL-01-015 | 1,810 | 0.10 | 1.6 | 0.73 | 0.58 | |
| KVL-01-016 | 5,662 | 0.07 | 1.2 | | | |
| KVL-01-018 | 5,906 | 0.07 | 1.1 | | | 170 |
| KVL-01-019 | 895 | 0.13 | 2.0 | 0.57 | 0.53 | 43.5 |
| KVL-01-020 | 2,513 | 0.09 | 1.5 | | | 170 |
| KVL-01-021 | 2,162 | 0.10 | 1.5 | | | |
| KVL-01-022 | 5,790 | 0.07 | 1.2 | | | 131 |
| KVL-01-025 | 1,363 | 0.11 | 1.8 | 0.72 | 0.54 | |
| KVL-01-026 | 5,271 | 0.08 | 1.2 | | | 172 |
| KVL-01-027 | 418 | 0.16 | 2.5 | 0.81 | 0.56 | |
| KVL-01-028 | 1,492 | 0.11 | 1.7 | 0.82 | 0.64 | |
| KVL-01-029 | 43,248 | 0.04 | 0.6 | | | |
| KVL-01-030 | 9,931 | 0.06 | 1.0 | | | |
| KVL-01-031 | na | | | | | |
| KVL-01-032 | na | | | | | |
| KVL-01-033 | na | | | | | |
| KVL-01-034 | 178 | 0.21 | 1.8 | 1.12 | 2.77 | |
| KVL-01-035 | 683 | 0.14 | 1.2 | | | |
| KVL-01-036 | 376 | 0.17 | 1.5 | 1.29 | 2.79 | |
| KVL-01-037 | 1,013 | 0.12 | 1.1 | | | |
| KVL-01-039 | 502 | 0.15 | 1.3 | 0.99 | 1.32 | |
| KVL-01-040 | 375 | 0.17 | 1.5 | 1.28 | 2.48 | |
| KVL-01-041 | 207 | 0.20 | 1.8 | 1.25 | 3.34 | |
| KVL-01-042 | 177 | 0.21 | 1.8 | 1.08 | 2.89 | |
| KVL-01-043 | 359 | 0.17 | 1.4 | 1.58 | 1.71 | |
| KVL-01-044 | 85 | 0.26 | 2.1 | | | |
| KVL-01-045 | 41 | 0.33 | 2.7 | | | |
| KVL-01-046 | 223 | 0.20 | 1.6 | | | |
| KVL-01-047 | na | | | | | |
| KVL-01-048 | 106 | 0.25 | 2.0 | | | 12.2 |
| KVL-01-049 | 29 | 0.36 | 2.9 | 1.20 | 2.51 | 8.9 |
| KVL-01-050 | 61 | 0.29 | 2.4 | | | 12.4 |
| KVL-01-051 | 39 | 0.33 | 2.7 | | | |
| KVL-01-052 | 57 | 0.30 | 2.4 | | | 15.5 |
| KVL-01-053 | 46 | 0.32 | 2.6 | | | |
| KVL-01-054 | 5,126 | 0.08 | 0.6 | 0.33 | 0.01 | |
| KVL-01-055 | 40 | 0.33 | 2.7 | | | |
| KVL-01-056 | 223 | 0.20 | 1.6 | | | 18.7 |
| KVL-01-057 | 33 | 0.35 | 2.9 | | | |
| KVL-01-058 | 35 | 0.34 | 2.8 | | | |
| KVL-01-059 | 382 | 0.17 | 1.4 | | | |
| KVL-01-060 | 62 | 0.29 | 2.4 | 0.96 | 1.92 | 18.9 |
| KVL-01-061 | 17 | 0.43 | 3.5 | 1.03 | 2.29 | |
| KVL-01-068 | 7,155 | 0.07 | 1.8 | | | 59.0 |
| KVL-01-069 | 1,831 | 0.10 | 2.7 | 0.84 | 1.05 | 63.2 |
| KVL-01-070 | 820 | 0.13 | 3.5 | 0.90 | 1.22 | 22.6 |
| KVL-01-071 | 738 | 0.14 | 3.6 | 0.99 | 1.47 | 14.2 |
| KVL-01-072 | 426,145 | 0.02 | 0.5 | | | 37 |
| KVL-01-073 | 7 | 0.56 | 3.4 | 2.00 | 2.23 | |
| KVL-01-074 | 1,466 | 0.11 | 2.9 | 0.86 | 1.48 | 2.3 |
| KVL-01-075 | 434 | 0.16 | 4.2 | 1.10 | 2.00 | 9.7 |
| KVL-01-076 | 656 | 0.14 | 3.7 | 1.17 | 2.59 | 1.7 |
| KVL-01-077 | 11 | 0.49 | 3.0 | 1.59 | 3.37 | |
| KVL-01-078 | | | | 1.41 | 9.46 | |
| KVL-01-079 | | | | 1.49 | 9.57 | |
| KVL-01-080 | | | | 1.39 | 7.29 | |
| KVL-01-081 | | | | 1.29 | 7.84 | |
| KVL-01-082 | | | | 1.56 | 11.85 | |
| KVL-01-083 | | | | 1.63 | 11.38 | |
| KVL-01-084 | | | | 1.45 | 10.65 | |
| KVL-01-085 | | | | 1.36 | 9.10 | |
| KVL-01-086 | | | | 1.52 | 10.76 | |
| KVL-01-087 | | | | 1.60 | 11.37 | |
| KVL-01-088 | | | | 1.38 | 8.35 | |
| KVL-01-089 | | | | 1.14 | 4.60 | |
| KVL-01-090 | 107 | 0.25 | 2.1 | 1.52 | 2.78 | |
| KVL-01-092 | 1,452 | 0.11 | 0.9 | 0.40 | 0.40 | |
| KVL-01-093 | 396,763 | 0.02 | 0.2 | 0.01 | 0.00 | |
| KVL-01-094 | 94 | 0.25 | 2.1 | 1.15 | 2.69 | |
| KVL-01-095 | 4,025 | 0.08 | 0.7 | 0.58 | 0.25 | |
| KVL-01-096 | 430 | 0.16 | 1.3 | 0.93 | 1.07 | |
| KVL-01-097 | 147 | 0.22 | 1.9 | 1.27 | 2.02 | |
| KVL-01-098 | 1,050 | 0.12 | 1.0 | 0.33 | 0.37 | |
| KVL-01-099 | 78 | 0.27 | 2.3 | 1.54 | 2.71 | |
| KVL-01-101 | 857 | 0.13 | 1.1 | 0.18 | 0.19 | |
| KVL-01-103 | 61 | 0.29 | 2.4 | 1.09 | 2.55 | |
| KVL-01-104 | 609 | 0.15 | 1.2 | 0.20 | 0.16 | |
| KVL-01-105 | | | | 1.44 | 8.91 | |
| KVL-01-106 | | | | 1.26 | 3.82 | |
| KVL-01-107 | | | | 1.25 | 5.36 | |
| KVL-01-108 | | | | 1.51 | 12.34 | |
| KVL-01-109 | | | | 1.48 | 10.25 | |
| KVL-01-110 | | | | 1.47 | 4.08 | |
| KVL-01-111 | | | | 1.47 | 8.31 | |
| KVL-01-112 | | | | 1.55 | 12.10 | |
| KVL-01-113 | | | | 1.48 | 8.20 | |
| KVL-01-114 | 134 | 0.23 | 1.9 | 0.67 | 1.40 | |
| KVL-01-115 | 610 | 0.15 | 1.2 | 0.42 | 0.73 | |
| KVL-01-117 | 108 | 0.24 | 1.5 | 1.20 | 1.13 | |
| KVL-01-118 | 325 | 0.18 | 1.1 | 0.86 | 0.72 | |
| KVL-01-119 | 83 | 0.26 | 1.6 | 1.78 | 2.62 | |
| KVL-01-120 | 249 | 0.19 | 1.2 | 1.28 | 1.54 | |
| KVL-01-121 | 257 | 0.19 | 1.1 | 0.77 | 0.54 | |
| KVL-01-122 | 262 | 0.19 | 1.1 | 1.58 | 1.07 | 144 |
| KVL-01-123 | 20 | 0.41 | 2.5 | 1.77 | 3.47 | |
| KVL-01-124 | 70 | 0.28 | 1.7 | 1.56 | 2.29 | |
| KVL-01-125 | 125 | 0.23 | 1.4 | 1.16 | 1.50 | |
| KVL-01-126 | 45 | 0.32 | 1.9 | 1.79 | 2.74 | |
| KVL-01-128 | 49 | 0.31 | 1.9 | 2.25 | 4.34 | |
| KVL-01-129 | 28 | 0.37 | 2.2 | 2.35 | 5.92 | |
| KVL-01-WT | 1,161 | 0.12 | 1.0 | 1.00 | 1.00 | 144 |

### Material and Methods for Example 6

### Lentiviral Supernatant Production

HEK293T (ATCC) cells are plated 5-6 hours before transfection in DMEM supplemented with 5% FBS and 50 ug/ml gentamycin in 6-well plates previously coated with poly-L-lysine. When the cells are completely attached to the plastic, they are transfected by adding a mixture of X-tremeGene™ 9 (Merck), helper plasmids pALD-Rev-K, pALD-GagPol-K, and pALD-VSV-G-K (Aldevron) and lug of plasmid of interest, or nuclease free H₂O for the mock control. Cells are first incubated for 16-18 hours at 37° C. 5% CO₂ before the medium is changed to DMEM supplemented with 5% FBS and 50 ug/ml gentamycin. After another 48 hours of incubation at 37° C. 5% CO2 the supernatant is filtered through a 0.45 μm syringe filter and stored at +4° C. until use.

### Activation of PBMC

HLA-A2 negative PBMCs are thawed and plated in X-vivo15 (Lonza) supplemented with 10% CTS™ Immune Cell SR (Thermo Fisher) (from now on called complete X-vivo15) and 100 IU/ml of IL-2 (Peprotech), at a density of 1×10⁶/ml, 1 ml/well, in 24-well plates previously coated with α-CD3 and α-CD28 (both from Thermo Fisher). After 48 h of incubation the activated PBMC are collected, washed and counted.

Transduction of Tc with Viral Supernatants

Activated PBMCs are plated in 6-well plates at a concentration of 1×10⁶/ml in complete X-vivo15 with 200 IU/ml of IL-2 and Lentiboost (Sirion Biotech) and mixed 1:1 with 1 ml of viral supernatants. The plates are then spun down at 900×g for 1 hour at 32° C. and incubated at 37° C. 5% CO₂ for 20h.

### Transduction Efficiency Check and T Cell Subculture

Transduced T cells are collected, washed, plated at 0.33×10⁶/ml in complete X-vivo15 with 50UI/ml IL-2, 2 ng/ml IL-7, and 2 ng/ml IL-15, and incubated at 37° C. 5% CO2. After 72 h a small aliquot of cells is used to assess the transduction efficiency by staining with fluorochrome conjugated, chain specific, monoclonal antibodies and acquired on a Cytoflex (Beckman Coulter) flow cytometer. The Tc culture is diluted approximately 1:4 with fresh complete X-vivo15 with 50UI/ml IL-2, 2 ng/ml IL-7, and 2 ng/ml IL-15 and incubated for 24 h until use in functional assays.

### Peptide Sensitivity and Potency Testing

To assess the sensitivity of the TCRs, transduced Tc are incubated at a 1:5 effector to target (E:T) ratio with T2 cells (DSMZ) pulsed with titrated amounts of peptide of interest (Genscript). IFNγ detection through ELISpot is used as readout. In summary, the day before the assay, 96-well filter plates with 0.45 μm pore size hydrophobic PVDF membrane (Merck) are activated with 35% ethanol and washed 4 times with sterile dH2O before addition of α-IFNγ coating antibody (ELISpot Flex: Human IFN-γ (ALP) kit (Mabtech)) and overnight incubation at +4° C. The day of the assay the coating antibody is washed off with PBS and the plates are blocked for 30 minutes at room temperature in complete Xvivo15. T2 cells are counted, resuspended at a concentration of 0.5×10⁶/ml, and incubated at 37° C. 5% CO₂ for at least 2 h with 12 titrated concentrations of target peptide, as controls unpulsed T2 cells and T2 cells pulsed with an irrelevant peptide are used. At the end of the incubation the pulsed T2 cells are plated in triplicated in the conditioned filter plates.

To assess potency, the TCR transduced Tc are incubated with cancer cell lines expressing HLA-A2 and the TCR target antigen at 2 E:T ratios: 1:5 and 1:10. IFNγ detection through ELISpot is used as readout. In summary, filter plates are prepared as described in the previous paragraph. NCI-H1703, C33A, and control cell lines (all from ATCC) are harvested and resuspended at a concentration of 0.5×10⁶/ml in complete Xvivo15 and plated twice in triplicates in the conditioned plates.

Effector cells are counted and resuspended in complete Xvivo15 without any cytokine. To allow resting of the cells before the functional assay, Tc are incubated alone for at least 3 hours before adding them to the plates containing the target cells. The % of TCR positive in each condition is normalized to the condition with the lowest expression by diluting the cells with Mock transduced Tc. Normalized Tc are then resuspended at 0.1×10⁶/ml and plated in the plates with pulsed T2 cells and in the plates with cancer cell lines targets (only the 1:5 E:T condition). The remaining effector cells are diluted with complete Xvivo15 to a concentration of 0.05×10⁶/ml and added to the 1:10 E:T condition of the coculture with cancer cell lines. Plates are incubated overnight at 37° C. 5% CO₂. The detection and development of the plates is performed using the ELISpot Flex: Human IFN-γ (ALP) kit (Mabtech) and following the manufacturer instructions.

### Example 7—Further Screening of Variants

Further TCRs were screened. The candidate TCRs tested are listed in Table 6 as KVL-01-145, KVL-01-149, KVL-01-151, and KVL-01-153.

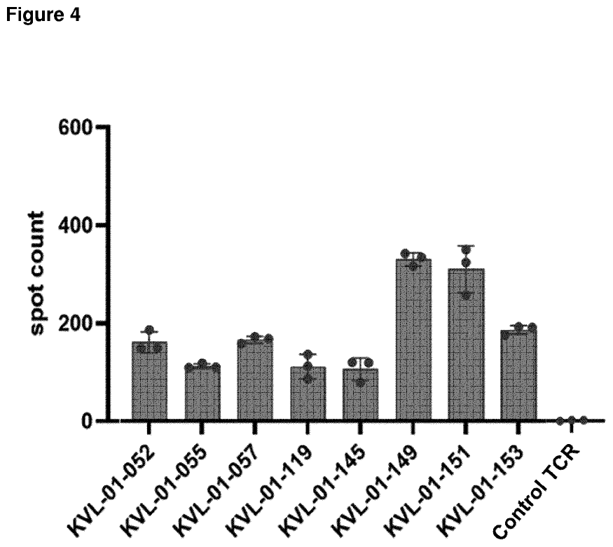

The results of the screening are shown in FIG. 4, which illustrates the results of an IFNγ ELISPOT assay. The target cell line in said assay was NCI-H1703 (MAGE-A4+ and HLA-A2+). Further details of the methodology of the IFNγ ELISPOT assay are provided for Example 6.

1. A T cell receptor (TCR), or an antigen-binding portion thereof, capable of binding specifically to a peptide according to SEQ ID NO: 122 presented in the context of an MHC class I molecule, comprising:
a TCR-alpha (TCR-a) variable region, or portion thereof, and a TCR-beta (TCR-b) variable region, or portion thereof, wherein
the TCR-a variable region, or portion thereof, comprises a complementarity determining region 3 alpha (CDR3a) comprising SEQ ID NO: 49 or SEQ ID NO: 53, wherein the CDR3a does not comprise SEQ ID NO: 7;
and/or the TCR-b variable region, or portion thereof, comprises a complementarity determining region 3 beta (CDR3b) comprising SEQ ID NO: 62 or SEQ ID NO: 64, wherein the CDR3b does not comprise SEQ ID NO: 14.

2. The TCR, or antigen-binding portion thereof, of claim 1, wherein
the CDR3a comprises SEQ ID NO: 49 or SEQ ID NO: 53 and does not comprise SEQ ID NO: 7, and
the TCR-b variable region, or portion thereof, comprises a CDR3b comprising any one of SEQ ID NOs: 14 and 57 to 66.

3. The TCR, or antigen-binding portion thereof, of claim 2, wherein
the CDR3a comprises SEQ ID NO: 55, and
the CDR3b comprises any one of SEQ ID NOs: 14 and 57 to 61.

4. The TCR, or antigen-binding portion thereof, of claim 1, wherein
the TCR-a variable region, or portion thereof, comprises a CDR3a comprising any one of SEQ ID NOs: 7, 26 to 55, 140, and 141 or optionally any one of SEQ ID NOs: 7 and 26 to 55, and
the CDR3b comprises SEQ ID NO: 62 or SEQ ID NO: 64, wherein the CDR3b does not comprise SEQ ID NO: 14.

5. The TCR, or antigen-binding portion thereof, of claim 4, wherein
the CDR3a comprises any one of SEQ ID NOs: 7, 26 to 48, 140, and 141 or optionally any one of SEQ ID NOs: 7 and 26 to 48, and
the CDR3b comprises SEQ ID NO: 66.

6. The TCR, or antigen-binding portion thereof, of any one of claims 1 to 5, wherein the TCR-a variable region, or portion thereof, comprises a CDR1a comprising any one of SEQ ID NOs: 5 and 15 to 17.

7. The TCR, or antigen-binding portion thereof, of any one of claims 1 to 6, wherein the TCR-a variable region, or portion thereof, comprises a CDR2a comprising any one of SEQ ID NOs: 6 and 18 to 25.

8. The TCR, or antigen-binding portion thereof, of any one of claims 1 to 7, wherein the TCR-b variable region, or portion thereof, comprises a CDR1b comprising SEQ ID NO: 12.

9. The TCR, or antigen-binding portion thereof, of any one of claims 1 to 8, wherein the TCR-b variable region, or portion thereof, comprises a CDR2b comprising SEQ ID NO: 13 or SEQ ID NO: 56.

10. The TCR, or antigen-binding portion thereof, of claim 1, wherein
the CDR3a comprises SEQ ID NO: 49 or SEQ ID NO: 53 and does not comprise SEQ ID NO: 7,
the TCR-a variable region, or portion thereof, comprises a CDR1a comprising any one of SEQ ID NOs: 5 and 15 to 17 and a CDR2a comprising any one of SEQ ID NOs: 6 and 18 to 25, and
the TCR-b variable region, or portion thereof, comprises a CDR1b comprising SEQ ID NO: 12, a CDR2b comprising SEQ ID NO: 13 or SEQ ID NO: 56, and a CDR3b comprising any one of SEQ ID NOs: 14 and 57 to 61.

11. The TCR, or antigen-binding portion thereof, of claim 1, wherein
the TCR-a variable region, or portion thereof, comprises a CDR1a comprising any one of SEQ ID NOs: 5 and 15 to 17, a CDR2a comprising any one of SEQ ID NOs: 6 and 18 to 25, and a CDR3a comprising any one of SEQ ID NOs: 7, 26 to 48, 140, and 141 or optionally any one of SEQ ID NOs: 7 and 26 to 48; and
the TCR-b variable region, or portion thereof, comprises a CDR1b comprising SEQ ID NO: 12 and comprises a CDR2b comprising SEQ ID NO: 13 or SEQ ID NO: 56; and
the CDR3b comprises SEQ ID NO: 62 or SEQ ID NO: 64, wherein the CDR3b does not comprise SEQ ID NO: 14.

12. A TCR, or an antigen-binding portion thereof, capable of binding specifically to a peptide according to SEQ ID NO: 122 presented in the context of an MHC class I molecule, comprising:
a TCR-a variable region, or portion thereof, and a TCR-b variable region, or portion thereof, wherein
the TCR-a variable region, or portion thereof, comprises a CDR3a and the TCR-b variable region, or portion thereof, comprises a CDR3b;
the CDR3a comprises any one of the CDR3a sequences recited in Table 1;
the CDR3b comprises any one of the CDR3b sequences recited in Table 2; and
the CDR3a and CDR3b are associated with the same TCR ID listed in Tables 1 and 2.

13. A TCR, or an antigen-binding portion thereof, capable of binding specifically to a peptide according to SEQ ID NO: 122 presented in the context of an MHC class I molecule, comprising:
a TCR-a variable region, or portion thereof, and a TCR-b variable region, or portion thereof, wherein
the TCR-a variable region, or portion thereof, comprises a CDR1a, a CDR2a, and a CDR3a and the TCR-b variable region, or portion thereof, comprises a CDR1b, a CDR2b, and a CDR3b; and
the CDR1a comprises any one of the CDR1a sequences recited in Table 1;
the CDR2a comprises any one of the CDR2a sequences recited in Table 1;
the CDR3a comprises any one of the CDR3a sequences recited in Table 1;
the CDR1b comprises any one of the CDR1b sequences recited in Table 2;
the CDR2b comprises any one of the CDR2b sequences recited in Table 2;
the CDR3b comprises any one of the CDR3b sequences recited in Table 2; and
the CDR1a, CDR2a, CDR3a, CDR1b, CDR2b, and CDR3b are all associated with the same TCR ID listed in Tables 1 and 2 (i.e. derived from the same clone).

14. A TCR, or an antigen-binding portion thereof, capable of binding specifically to a peptide according to SEQ ID NO: 122 presented in the context of an MHC class I molecule, comprising:
a TCR-a variable region, or portion thereof, and a TCR-b variable region, or portion thereof, wherein
(1) the TCRa variable region, or portion thereof, comprises a CDR3a comprising or according to SEQ ID NO: 7 or SEQ ID NO: 26 and the TCRb variable region, or portion thereof, comprises a CDR3b comprising or according SEQ ID NO: 14 or SEQ ID NO: 57; or
(2) the TCRa variable region, or portion thereof, comprises a CDR3a comprising or according to SEQ ID NO: 7 or SEQ ID NO: 26 and the TCRb variable region, or portion thereof, comprises a CDR3b comprising or according SEQ ID NO: 14 or SEQ ID NO: 57; or
(3) the TCRa variable region, or portion thereof, comprises a CDR3a comprising or according to SEQ ID NO: 7 or SEQ ID NO: 26 and the TCRb variable region, or portion thereof, comprises a CDR3b comprising or according SEQ ID NO: 14 or SEQ ID NO: 57; or
(4) the TCRa variable region, or portion thereof, comprises a CDR3a comprising or according to SEQ ID NO: 7 or SEQ ID NO: 26 and the TCRb variable region, or portion thereof, comprises a CDR3b comprising or according SEQ ID NO: 14 or SEQ ID NO: 57; or
(5) the TCRa variable region, or portion thereof, comprises a CDR3a comprising or according to SEQ ID NO: 27 or SEQ ID NO: 28 and the TCRb variable region, or portion thereof, comprises a CDR3b comprising or according SEQ ID NO: 14 or SEQ ID NO: 57; or
(6) the TCRa variable region, or portion thereof, comprises a CDR3a comprising or according to SEQ ID NO: 29 or SEQ ID NO: 30 and the TCRb variable region, or portion thereof, comprises a CDR3b comprising or according SEQ ID NO: 14 or SEQ ID NO: 57; or
(7) the TCRa variable region, or portion thereof, comprises a CDR3a comprising or according to SEQ ID NO: 31 or SEQ ID NO: 32 and the TCRb variable region, or portion thereof, comprises a CDR3b comprising or according SEQ ID NO: 14 or SEQ ID NO: 57; or
(8) the TCRa variable region, or portion thereof, comprises a CDR3a comprising or according to SEQ ID NO: 33 or SEQ ID NO: 34 and the TCRb variable region, or portion thereof, comprises a CDR3b comprising or according SEQ ID NO: 14 or SEQ ID NO: 57; or
(9) the TCRa variable region, or portion thereof, comprises a CDR3a comprising or according to SEQ ID NO: 29 or SEQ ID NO: 30 and the TCRb variable region, or portion thereof, comprises a CDR3b comprising or according SEQ ID NO: 14 or SEQ ID NO: 57; or
(10) the TCRa variable region, or portion thereof, comprises a CDR3a comprising or according to SEQ ID NO: 31 or SEQ ID NO: 32 and the TCRb variable region, or portion thereof, comprises a CDR3b comprising or according SEQ ID NO: 14 or SEQ ID NO: 57; or
(11) the TCRa variable region, or portion thereof, comprises a CDR3a comprising or according to SEQ ID NO: 29 or SEQ ID NO: 30 and the TCRb variable region, or portion thereof, comprises a CDR3b comprising or according SEQ ID NO: 14 or SEQ ID NO: 57; or
(12) the TCRa variable region, or portion thereof, comprises a CDR3a comprising or according to SEQ ID NO: 31 or SEQ ID NO: 32 and the TCRb variable region, or portion thereof, comprises a CDR3b comprising or according SEQ ID NO: 14 or SEQ ID NO: 57; or
(13) the TCRa variable region, or portion thereof, comprises a CDR3a comprising or according to SEQ ID NO: 7 or SEQ ID NO: 26 and the TCRb variable region, or portion thereof, comprises a CDR3b comprising or according SEQ ID NO: 58 or SEQ ID NO: 59; or
(14) the TCRa variable region, or portion thereof, comprises a CDR3a comprising or according to SEQ ID NO: 7 or SEQ ID NO: 26 and the TCRb variable region, or portion thereof, comprises a CDR3b comprising or according SEQ ID NO: 58 or SEQ ID NO: 59; or
(15) the TCRa variable region, or portion thereof, comprises a CDR3a comprising or according to SEQ ID NO: 7 or SEQ ID NO: 26 and the TCRb variable region, or portion thereof, comprises a CDR3b comprising or according SEQ ID NO: 58 or SEQ ID NO: 59; or
(16) the TCRa variable region, or portion thereof, comprises a CDR3a comprising or according to SEQ ID NO: 7 or SEQ ID NO: 26 and the TCRb variable region, or portion thereof, comprises a CDR3b comprising or according SEQ ID NO: 58 or SEQ ID NO: 59; or
(17) the TCRa variable region, or portion thereof, comprises a CDR3a comprising or according to SEQ ID NO: 27 or SEQ ID NO: 28 and the TCRb variable region, or portion thereof, comprises a CDR3b comprising or according SEQ ID NO: 58 or SEQ ID NO: 59; or
(18) the TCRa variable region, or portion thereof, comprises a CDR3a comprising or according to SEQ ID NO: 7 or SEQ ID NO: 26 and the TCRb variable region, or portion thereof, comprises a CDR3b comprising or according SEQ ID NO: 58 or SEQ ID NO: 59; or
(19) the TCRa variable region, or portion thereof, comprises a CDR3a comprising or according to SEQ ID NO: 31 or SEQ ID NO: 32 and the TCRb variable region, or portion thereof, comprises a CDR3b comprising or according SEQ ID NO: 58 or SEQ ID NO: 59; or
(20) the TCRa variable region, or portion thereof, comprises a CDR3a comprising or according to SEQ ID NO: 35 or SEQ ID NO: 36 and the TCRb variable region, or portion thereof, comprises a CDR3b comprising or according SEQ ID NO: 14 or SEQ ID NO: 57; or
(21) the TCRa variable region, or portion thereof, comprises a CDR3a comprising or according to SEQ ID NO: 37 or SEQ ID NO: 38 and the TCRb variable region, or portion thereof, comprises a CDR3b comprising or according SEQ ID NO: 14 or SEQ ID NO: 57; or
(22) the TCRa variable region, or portion thereof, comprises a CDR3a comprising or according to SEQ ID NO: 39 or SEQ ID NO: 40 and the TCRb variable region, or portion thereof, comprises a CDR3b comprising or according SEQ ID NO: 14 or SEQ ID NO: 57; or
(23) the TCRa variable region, or portion thereof, comprises a CDR3a comprising or according to SEQ ID NO: 41 or SEQ ID NO: 42 and the TCRb variable region, or portion thereof, comprises a CDR3b comprising or according SEQ ID NO: 14 or SEQ ID NO: 57; or
(24) the TCRa variable region, or portion thereof, comprises a CDR3a comprising or according to SEQ ID NO: 35 or SEQ ID NO: 36 and the TCRb variable region, or portion thereof, comprises a CDR3b comprising or according SEQ ID NO: 58 or SEQ ID NO: 59; or
(25) the TCRa variable region, or portion thereof, comprises a CDR3a comprising or according to SEQ ID NO: 37 or SEQ ID NO: 38 and the TCRb variable region, or portion thereof, comprises a CDR3b comprising or according SEQ ID NO: 58 or SEQ ID NO: 59; or
(26) the TCRa variable region, or portion thereof, comprises a CDR3a comprising or according to SEQ ID NO: 39 or SEQ ID NO: 40 and the TCRb variable region, or portion thereof, comprises a CDR3b comprising or according SEQ ID NO: 58 or SEQ ID NO: 59; or
(27) the TCRa variable region, or portion thereof, comprises a CDR3a comprising or according to SEQ ID NO: 41 or SEQ ID NO: 42 and the TCRb variable region, or portion thereof, comprises a CDR3b comprising or according SEQ ID NO: 58 or SEQ ID NO: 59; or
(28) the TCRa variable region, or portion thereof, comprises a CDR3a comprising or according to SEQ ID NO: 43 or SEQ ID NO: 44 and the TCRb variable region, or portion thereof, comprises a CDR3b comprising or according SEQ ID NO: 58 or SEQ ID NO: 59; or
(29) the TCRa variable region, or portion thereof, comprises a CDR3a comprising or according to SEQ ID NO: 29 or SEQ ID NO: 30 and the TCRb variable region, or portion thereof, comprises a CDR3b comprising or according SEQ ID NO: 14 or SEQ ID NO: 57; or
(30) the TCRa variable region, or portion thereof, comprises a CDR3a comprising or according to SEQ ID NO: 29 or SEQ ID NO: 30 and the TCRb variable region, or portion thereof, comprises a CDR3b comprising or according SEQ ID NO: 14 or SEQ ID NO: 57; or
(31) the TCRa variable region, or portion thereof, comprises a CDR3a comprising or according to SEQ ID NO: 31 or SEQ ID NO: 32 and the TCRb variable region, or portion thereof, comprises a CDR3b comprising or according SEQ ID NO: 14 or SEQ ID NO: 57; or
(32) the TCRa variable region, or portion thereof, comprises a CDR3a comprising or according to SEQ ID NO: 31 or SEQ ID NO: 32 and the TCRb variable region, or portion thereof, comprises a CDR3b comprising or according SEQ ID NO: 14 or SEQ ID NO: 57; or
(33) the TCRa variable region, or portion thereof, comprises a CDR3a comprising or according to SEQ ID NO: 31 or SEQ ID NO: 32 and the TCRb variable region, or portion thereof, comprises a CDR3b comprising or according SEQ ID NO: 14 or SEQ ID NO: 57; or
(34) the TCRa variable region, or portion thereof, comprises a CDR3a comprising or according to SEQ ID NO: 7 or SEQ ID NO: 26 and the TCRb variable region, or portion thereof, comprises a CDR3b comprising or according SEQ ID NO: 14 or SEQ ID NO: 57; or
(35) the TCRa variable region, or portion thereof, comprises a CDR3a comprising or according to SEQ ID NO: 31 or SEQ ID NO: 32 and the TCRb variable region, or portion thereof, comprises a CDR3b comprising or according SEQ ID NO: 14 or SEQ ID NO: 57; or
(36) the TCRa variable region, or portion thereof, comprises a CDR3a comprising or according to SEQ ID NO: 31 or SEQ ID NO: 32 and the TCRb variable region, or portion thereof, comprises a CDR3b comprising or according SEQ ID NO: 14 or SEQ ID NO: 57; or
(37) the TCRa variable region, or portion thereof, comprises a CDR3a comprising or according to SEQ ID NO: 31 or SEQ ID NO: 32 and the TCRb variable region, or portion thereof, comprises a CDR3b comprising or according SEQ ID NO: 14 or SEQ ID NO: 57; or
(38) the TCRa variable region, or portion thereof, comprises a CDR3a comprising or according to SEQ ID NO: 31 or SEQ ID NO: 32 and the TCRb variable region, or portion thereof, comprises a CDR3b comprising or according SEQ ID NO: 14 or SEQ ID NO: 57; or
(39) the TCRa variable region, or portion thereof, comprises a CDR3a comprising or according to SEQ ID NO: 31 or SEQ ID NO: 32 and the TCRb variable region, or portion thereof, comprises a CDR3b comprising or according SEQ ID NO: 14 or SEQ ID NO: 57; or
(40) the TCRa variable region, or portion thereof, comprises a CDR3a comprising or according to SEQ ID NO: 29 or SEQ ID NO: 30 and the TCRb variable region, or portion thereof, comprises a CDR3b comprising or according SEQ ID NO: 14 or SEQ ID NO: 57; or
(41) the TCRa variable region, or portion thereof, comprises a CDR3a comprising or according to SEQ ID NO: 29 or SEQ ID NO: 30 and the TCRb variable region, or portion thereof, comprises a CDR3b comprising or according SEQ ID NO: 14 or SEQ ID NO: 57; or
(42) the TCRa variable region, or portion thereof, comprises a CDR3a comprising or according to SEQ ID NO: 29 or SEQ ID NO: 30 and the TCRb variable region, or portion thereof, comprises a CDR3b comprising or according SEQ ID NO: 14 or SEQ ID NO: 57; or
(43) the TCRa variable region, or portion thereof, comprises a CDR3a comprising or according to SEQ ID NO: 45 or SEQ ID NO: 46 and the TCRb variable region, or portion thereof, comprises a CDR3b comprising or according SEQ ID NO: 14 or SEQ ID NO: 57; or
(44) the TCRa variable region, or portion thereof, comprises a CDR3a comprising or according to SEQ ID NO: 47 or SEQ ID NO: 48 and the TCRb variable region, or portion thereof, comprises a CDR3b comprising or according SEQ ID NO: 14 or SEQ ID NO: 57; or
(45) the TCRa variable region, or portion thereof, comprises a CDR3a comprising or according to SEQ ID NO: 45 or SEQ ID NO: 46 and the TCRb variable region, or portion thereof, comprises a CDR3b comprising or according SEQ ID NO: 58 or SEQ ID NO: 59; or
(46) the TCRa variable region, or portion thereof, comprises a CDR3a comprising or according to SEQ ID NO: 45 or SEQ ID NO: 46 and the TCRb variable region, or portion thereof, comprises a CDR3b comprising or according SEQ ID NO: 14 or SEQ ID NO: 57; or
(47) the TCRa variable region, or portion thereof, comprises a CDR3a comprising or according to SEQ ID NO: 47 or SEQ ID NO: 48 and the TCRb variable region, or portion thereof, comprises a CDR3b comprising or according SEQ ID NO: 14 or SEQ ID NO: 57; or
(48) the TCRa variable region, or portion thereof, comprises a CDR3a comprising or according to SEQ ID NO: 31 or SEQ ID NO: 32 and the TCRb variable region, or portion thereof, comprises a CDR3b comprising or according SEQ ID NO: 14 or SEQ ID NO: 57; or
(49) the TCRa variable region, or portion thereof, comprises a CDR3a comprising or according to SEQ ID NO: 31 or SEQ ID NO: 32 and the TCRb variable region, or portion thereof, comprises a CDR3b comprising or according SEQ ID NO: 14 or SEQ ID NO: 57; or
(50) the TCRa variable region, or portion thereof, comprises a CDR3a comprising or according to SEQ ID NO: 31 or SEQ ID NO: 32 and the TCRb variable region, or portion thereof, comprises a CDR3b comprising or according SEQ ID NO: 60 or SEQ ID NO: 61; or
(51) the TCRa variable region, or portion thereof, comprises a CDR3a comprising or according to SEQ ID NO: 140 or SEQ ID NO: 141 and the TCRb variable region, or portion thereof, comprises a CDR3b comprising or according SEQ ID NO: 14 or SEQ ID NO: 57.

15. A TCR, or an antigen-binding portion thereof, capable of binding specifically to a peptide according to SEQ ID NO: 122 presented in the context of an MHC class I molecule, comprising:
a TCR-a variable region, or portion thereof, and a TCR-b variable region, or portion thereof, wherein
(1) the TCRa variable region, or portion thereof, comprises a CDR1a, CDR2a, and CDR3a comprising or according to SEQ ID NOs: 5, 18, and 7/26 respectively and the TCRb variable region, or portion thereof, comprises a CDR1b, CDR2b, and CDR3b comprising or according to SEQ ID NOs: 12, 13, 14/57 respectively; or
(2) the TCRa variable region, or portion thereof, comprises a CDR1a, CDR2a, and CDR3a comprising or according to SEQ ID NOs: 5, 19, 7/26 respectively and the TCRb variable region, or portion thereof, comprises a CDR1b, CDR2b, and CDR3b comprising or according to SEQ ID NOs: 12, 13, 14/57 respectively; or
(3) the TCRa variable region, or portion thereof, comprises a CDR1a, CDR2a, and CDR3a comprising or according to SEQ ID NOs: 5, 20, 7/26 respectively and the TCRb variable region, or portion thereof, comprises a CDR1b, CDR2b, and CDR3b comprising or according to SEQ ID NOs: 12, 13, 14/57 respectively; or
(4) the TCRa variable region, or portion thereof, comprises a CDR1a, CDR2a, and CDR3a comprising or according to SEQ ID NOs: 5, 21, 7/26 respectively and the TCRb variable region, or portion thereof, comprises a CDR1b, CDR2b, and CDR3b comprising or according to SEQ ID NOs: 12, 13, 14/57 respectively; or
(5) the TCRa variable region, or portion thereof, comprises a CDR1a, CDR2a, and CDR3a comprising or according to SEQ ID NOs: 5, 6, 27/28 respectively and the TCRb variable region, or portion thereof, comprises a CDR1b, CDR2b, and CDR3b comprising or according to SEQ ID NOs: 12, 13, 14/57 respectively; or
(6) the TCRa variable region, or portion thereof, comprises a CDR1a, CDR2a, and CDR3a comprising or according to SEQ ID NOs: 15, 6, 29/30 respectively and the TCRb variable region, or portion thereof, comprises a CDR1b, CDR2b, and CDR3b comprising or according to SEQ ID NOs: 12, 13, 14/57 respectively; or
(7) the TCRa variable region, or portion thereof, comprises a CDR1a, CDR2a, and CDR3a comprising or according to SEQ ID NOs: 5, 6, 31/32 respectively and the TCRb variable region, or portion thereof, comprises a CDR1b, CDR2b, and CDR3b comprising or according to SEQ ID NOs: 12, 13, 14/57 respectively; or
(8) the TCRa variable region, or portion thereof, comprises a CDR1a, CDR2a, and CDR3a comprising or according to SEQ ID NOs: 15, 6, 33/34 respectively and the TCRb variable region, or portion thereof, comprises a CDR1b, CDR2b, and CDR3b comprising or according to SEQ ID NOs: 12, 13, 14/57 respectively; or
(9) the TCRa variable region, or portion thereof, comprises a CDR1a, CDR2a, and CDR3a comprising or according to SEQ ID NOs: 5, 6, 29/30 respectively and the TCRb variable region, or portion thereof, comprises a CDR1b, CDR2b, and CDR3b comprising or according to SEQ ID NOs: 12, 13, 14/57 respectively; or
(10) the TCRa variable region, or portion thereof, comprises a CDR1a, CDR2a, and CDR3a comprising or according to SEQ ID NOs: 15, 6, 31/32 respectively and the TCRb variable region, or portion thereof, comprises a CDR1b, CDR2b, and CDR3b comprising or according to SEQ ID NOs: 12, 13, 14/57 respectively; or
(11) the TCRa variable region, or portion thereof, comprises a CDR1a, CDR2a, and CDR3a comprising or according to SEQ ID NOs: 15, 6, 29/30 respectively and the TCRb variable region, or portion thereof, comprises a CDR1b, CDR2b, and CDR3b comprising or according to SEQ ID NOs: 12, 13, 14/57 respectively; or
(12) the TCRa variable region, or portion thereof, comprises a CDR1a, CDR2a, and CDR3a comprising or according to SEQ ID NOs: 5, 6, 31/32 respectively and the TCRb variable region, or portion thereof, comprises a CDR1b, CDR2b, and CDR3b comprising or according to SEQ ID NOs: 12, 13, 14/57 respectively; or
(13) the TCRa variable region, or portion thereof, comprises a CDR1a, CDR2a, and CDR3a comprising or according to SEQ ID NOs: 5, 18, 7/26 respectively and the TCRb variable region, or portion thereof, comprises a CDR1b, CDR2b, and CDR3b comprising or according to SEQ ID NOs: 12, 13, 58/59 respectively; or
(14) the TCRa variable region, or portion thereof, comprises a CDR1a, CDR2a, and CDR3a comprising or according to SEQ ID NOs: 5, 19, 7/26 respectively and the TCRb variable region, or portion thereof, comprises a CDR1b, CDR2b, and CDR3b comprising or according to SEQ ID NOs: 12, 13, 58/59 respectively; or
(15) the TCRa variable region, or portion thereof, comprises a CDR1a, CDR2a, and CDR3a comprising or according to SEQ ID NOs: 5, 20, 7/26 respectively and the TCRb variable region, or portion thereof, comprises a CDR1b, CDR2b, and CDR3b comprising or according to SEQ ID NOs: 12, 13, 58/59 respectively; or
(16) the TCRa variable region, or portion thereof, comprises a CDR1a, CDR2a, and CDR3a comprising or according to SEQ ID NOs: 5, 21, 7/26 respectively and the TCRb variable region, or portion thereof, comprises a CDR1b, CDR2b, and CDR3b comprising or according to SEQ ID NOs: 12, 13, 58/59 respectively; or
(17) the TCRa variable region, or portion thereof, comprises a CDR1a, CDR2a, and CDR3a comprising or according to SEQ ID NOs: 5, 6, 27/28 respectively and the TCRb variable region, or portion thereof, comprises a CDR1b, CDR2b, and CDR3b comprising or according to SEQ ID NOs: 12, 13, 58/59 respectively; or
(18) the TCRa variable region, or portion thereof, comprises a CDR1a, CDR2a, and CDR3a comprising or according to SEQ ID NOs: 5, 6, 7/26 respectively and the TCRb variable region, or portion thereof, comprises a CDR1b, CDR2b, and CDR3b comprising or according to SEQ ID NOs: 12, 13, 58/59 respectively; or
(19) the TCRa variable region, or portion thereof, comprises a CDR1a, CDR2a, and CDR3a comprising or according to SEQ ID NOs: 5, 6, 31/32 respectively and the TCRb variable region, or portion thereof, comprises a CDR1b, CDR2b, and CDR3b comprising or according to SEQ ID NOs: 12, 13, 58/59 respectively; or
(20) the TCRa variable region, or portion thereof, comprises a CDR1a, CDR2a, and CDR3a comprising or according to SEQ ID NOs: 5, 6, 35/36 respectively and the TCRb variable region, or portion thereof, comprises a CDR1b, CDR2b, and CDR3b comprising or according to SEQ ID NOs: 12, 13, 14/57 respectively; or
(21) the TCRa variable region, or portion thereof, comprises a CDR1a, CDR2a, and CDR3a comprising or according to SEQ ID NOs: 5, 6, 37/38 respectively and the TCRb variable region, or portion thereof, comprises a CDR1b, CDR2b, and CDR3b comprising or according to SEQ ID NOs: 12, 13, 14/57 respectively; or
(22) the TCRa variable region, or portion thereof, comprises a CDR1a, CDR2a, and CDR3a comprising or according to SEQ ID NOs: 5, 6, 39/40 respectively and the TCRb variable region, or portion thereof, comprises a CDR1b, CDR2b, and CDR3b comprising or according to SEQ ID NOs: 12, 13, 14/57 respectively; or
(23) the TCRa variable region, or portion thereof, comprises a CDR1a, CDR2a, and CDR3a comprising or according to SEQ ID NOs: 5, 6, 41/42 respectively and the TCRb variable region, or portion thereof, comprises a CDR1b, CDR2b, and CDR3b comprising or according to SEQ ID NOs: 12, 13, 14/57 respectively; or
(24) the TCRa variable region, or portion thereof, comprises a CDR1a, CDR2a, and CDR3a comprising or according to SEQ ID NOs: 5, 6, 35/36 respectively and the TCRb variable region, or portion thereof, comprises a CDR1b, CDR2b, and CDR3b comprising or according to SEQ ID NOs: 12, 13, 58/59 respectively; or
(25) the TCRa variable region, or portion thereof, comprises a CDR1a, CDR2a, and CDR3a comprising or according to SEQ ID NOs: 5, 6, 37/38 respectively and the TCRb variable region, or portion thereof, comprises a CDR1b, CDR2b, and CDR3b comprising or according to SEQ ID NOs: 12, 13, 58/59 respectively; or
(26) the TCRa variable region, or portion thereof, comprises a CDR1a, CDR2a, and CDR3a comprising or according to SEQ ID NOs: 5, 6, 39/40 respectively and the TCRb variable region, or portion thereof, comprises a CDR1b, CDR2b, and CDR3b comprising or according to SEQ ID NOs: 12, 13, 58/59 respectively; or
(27) the TCRa variable region, or portion thereof, comprises a CDR1a, CDR2a, and CDR3a comprising or according to SEQ ID NOs: 5, 6, 41/42 respectively and the TCRb variable region, or portion thereof, comprises a CDR1b, CDR2b, and CDR3b comprising or according to SEQ ID NOs: 12, 13, 58/59 respectively; or
(28) the TCRa variable region, or portion thereof, comprises a CDR1a, CDR2a, and CDR3a comprising or according to SEQ ID NOs: 5, 6, 43/44 respectively and the TCRb variable region, or portion thereof, comprises a CDR1b, CDR2b, and CDR3b comprising or according to SEQ ID NOs: 12, 13, 58/59 respectively; or
(29) the TCRa variable region, or portion thereof, comprises a CDR1a, CDR2a, and CDR3a comprising or according to SEQ ID NOs: 15, 22, 29/30 respectively and the TCRb variable region, or portion thereof, comprises a CDR1b, CDR2b, and CDR3b comprising or according to SEQ ID NOs: 12, 13, 14/57 respectively; or
(30) the TCRa variable region, or portion thereof, comprises a CDR1a, CDR2a, and CDR3a comprising or according to SEQ ID NOs: 15, 23, 29/30 respectively and the TCRb variable region, or portion thereof, comprises a CDR1b, CDR2b, and CDR3b comprising or according to SEQ ID NOs: 12, 13, 14/57 respectively; or
(31) the TCRa variable region, or portion thereof, comprises a CDR1a, CDR2a, and CDR3a comprising or according to SEQ ID NOs: 15, 22, 31/32 respectively and the TCRb variable region, or portion thereof, comprises a CDR1b, CDR2b, and CDR3b comprising or according to SEQ ID NOs: 12, 13, 14/57 respectively; or
(32) the TCRa variable region, or portion thereof, comprises a CDR1a, CDR2a, and CDR3a comprising or according to SEQ ID NOs: 15, 23, 31/32 respectively and the TCRb variable region, or portion thereof, comprises a CDR1b, CDR2b, and CDR3b comprising or according to SEQ ID NOs: 12, 13, 14/57 respectively; or
(33) the TCRa variable region, or portion thereof, comprises a CDR1a, CDR2a, and CDR3a comprising or according to SEQ ID NOs: 15, 6, 31/32 respectively and the TCRb variable region, or portion thereof, comprises a CDR1b, CDR2b, and CDR3b comprising or according to SEQ ID NOs: 12, 13, 14/57 respectively; or
(34) the TCRa variable region, or portion thereof, comprises a CDR1a, CDR2a, and CDR3a comprising or according to SEQ ID NOs: 15, 6, 7/26 respectively and the TCRb variable region, or portion thereof, comprises a CDR1b, CDR2b, and CDR3b comprising or according to SEQ ID NOs: 12, 13, 14/57 respectively; or
(35) the TCRa variable region, or portion thereof, comprises a CDR1a, CDR2a, and CDR3a comprising or according to SEQ ID NOs: 16, 22, 31/32 respectively and the TCRb variable region, or portion thereof, comprises a CDR1b, CDR2b, and CDR3b comprising or according to SEQ ID NOs: 12, 13, 14/57 respectively; or
(36) the TCRa variable region, or portion thereof, comprises a CDR1a, CDR2a, and CDR3a comprising or according to SEQ ID NOs: 17, 23, 31/32 respectively and the TCRb variable region, or portion thereof, comprises a CDR1b, CDR2b, and CDR3b comprising or according to SEQ ID NOs: 12, 13, 14/57 respectively; or
(37) the TCRa variable region, or portion thereof, comprises a CDR1a, CDR2a, and CDR3a comprising or according to SEQ ID NOs: 17, 6, 31/32 respectively and the TCRb variable region, or portion thereof, comprises a CDR1b, CDR2b, and CDR3b comprising or according to SEQ ID NOs: 12, 13, 14/57 respectively; or
(38) the TCRa variable region, or portion thereof, comprises a CDR1a, CDR2a, and CDR3a comprising or according to SEQ ID NOs: 5, 23, 31/32 respectively and the TCRb variable region, or portion thereof, comprises a CDR1b, CDR2b, and CDR3b comprising or according to SEQ ID NOs: 12, 13, 14/57 respectively; or
(39) the TCRa variable region, or portion thereof, comprises a CDR1a, CDR2a, and CDR3a comprising or according to SEQ ID NOs: 5, 22, 31/32 respectively and the TCRb variable region, or portion thereof, comprises a CDR1b, CDR2b, and CDR3b comprising or according to SEQ ID NOs: 12, 13, 14/57 respectively; or
(40) the TCRa variable region, or portion thereof, comprises a CDR1a, CDR2a, and CDR3a comprising or according to SEQ ID NOs: 5, 6, 29/30 respectively and the TCRb variable region, or portion thereof, comprises a CDR1b, CDR2b, and CDR3b comprising or according to SEQ ID NOs: 12, 13, 14/57 respectively; or
(41) the TCRa variable region, or portion thereof, comprises a CDR1a, CDR2a, and CDR3a comprising or according to SEQ ID NOs: 5, 23, 29/30 respectively and the TCRb variable region, or portion thereof, comprises a CDR1b, CDR2b, and CDR3b comprising or according to SEQ ID NOs: 12, 13, 14/57 respectively; or
(42) the TCRa variable region, or portion thereof, comprises a CDR1a, CDR2a, and CDR3a comprising or according to SEQ ID NOs: 5, 22, 29/30 respectively and the TCRb variable region, or portion thereof, comprises a CDR1b, CDR2b, and CDR3b comprising or according to SEQ ID NOs: 12, 13, 14/57 respectively; or
(43) the TCRa variable region, or portion thereof, comprises a CDR1a, CDR2a, and CDR3a comprising or according to SEQ ID NOs: 5, 6, 45/46 respectively and the TCRb variable region, or portion thereof, comprises a CDR1b, CDR2b, and CDR3b comprising or according to SEQ ID NOs: 12, 13, 14/57 respectively; or
(44) the TCRa variable region, or portion thereof, comprises a CDR1a, CDR2a, and CDR3a comprising or according to SEQ ID NOs: 5, 19, 47/48 respectively and the TCRb variable region, or portion thereof, comprises a CDR1b, CDR2b, and CDR3b comprising or according to SEQ ID NOs: 12, 13, 14/57 respectively; or
(45) the TCRa variable region, or portion thereof, comprises a CDR1a, CDR2a, and CDR3a comprising or according to SEQ ID NOs: 5, 19, 45/46 respectively and the TCRb variable region, or portion thereof, comprises a CDR1b, CDR2b, and CDR3b comprising or according to SEQ ID NOs: 12, 13, 58/59 respectively; or
(46) the TCRa variable region, or portion thereof, comprises a CDR1a, CDR2a, and CDR3a comprising or according to SEQ ID NOs: 5, 19, 45/46 respectively and the TCRb variable region, or portion thereof, comprises a CDR1b, CDR2b, and CDR3b comprising or according to SEQ ID NOs: 12, 13, 14/57 respectively; or
(47) the TCRa variable region, or portion thereof, comprises a CDR1a, CDR2a, and CDR3a comprising or according to SEQ ID NOs: 5, 19, 47/48 respectively and the TCRb variable region, or portion thereof, comprises a CDR1b, CDR2b, and CDR3b comprising or according to SEQ ID NOs: 12, 13, 14/57 respectively; or
(48) the TCRa variable region, or portion thereof, comprises a CDR1a, CDR2a, and CDR3a comprising or according to SEQ ID NOs: 5, 19, 31/32 respectively and the TCRb variable region, or portion thereof, comprises a CDR1b, CDR2b, and CDR3b comprising or according to SEQ ID NOs: 12, 13, 14/57 respectively; or
(49) the TCRa variable region, or portion thereof, comprises a CDR1a, CDR2a, and CDR3a comprising or according to SEQ ID NOs: 5, 19, 31/32 respectively and the TCRb variable region, or portion thereof, comprises a CDR1b, CDR2b, and CDR3b comprising or according to SEQ ID NOs: 12, 56, 14/57 respectively; or
(50) the TCRa variable region, or portion thereof, comprises a CDR1a, CDR2a, and CDR3a comprising or according to SEQ ID NOs: 5, 6, 31/32 respectively and the TCRb variable region, or portion thereof, comprises a CDR1b, CDR2b, and CDR3b comprising or according to SEQ ID NOs: 12, 13, 60/61 respectively; or
(51) the TCRa variable region, or portion thereof, comprises a CDR1a, CDR2a, and CDR3a comprising or according to SEQ ID NOs: 5, 6, 140/141 respectively and the TCRb variable region, or portion thereof, comprises a CDR1b, CDR2b, and CDR3b comprising or according to SEQ ID NOs: 12, 13, 14/57 respectively; or
(52) the TCRa variable region, or portion thereof, comprises a CDR1a, CDR2a, and CDR3a comprising or according to SEQ ID NOs: 5, 22, 140/141 respectively and the TCRb variable region, or portion thereof, comprises a CDR1b, CDR2b, and CDR3b comprising or according to SEQ ID NOs: 12, 13, 14/57 respectively.

16. The TCR, or antigen-binding portion thereof, of any one of claims 1 to 15, wherein
the TCR-a variable region comprises a sequence at least 70%, 80%, 90%, 95%, or 100% identical to SEQ ID NO: 71, a sequence at least 70%, 80%, 90%, 95%, or 100% identical to SEQ ID NO: 72, a sequence at least 70%, 80%, 90%, 95%, or 100% identical to SEQ ID NO: 73, and a sequence at least 70%, 80%, 90%, 95%, or 100% identical to SEQ ID NO: 74; and/or
the TCR-b variable region comprises a sequence at least 70%, 80%, 90%, 95%, or 100% identical to SEQ ID NO: 75, a sequence at least 70%, 80%, 90%, 95%, or 100% identical to any one of SEQ ID NOs: 76 to 79, a sequence at least 70%, 80%, 90%, 95%, or 100% identical to SEQ ID NO: 80, and a sequence at least 70%, 80%, 90%, 95%, or 100% identical to SEQ ID NO: 81.

17. The TCR, or an antigen-binding portion thereof, of any of claims 1 to 16, wherein
the TCR-a variable region comprises a sequence that is at least 70%, 80%, 85%, 90%, 95%, 97%, 98%, 99%, or 100% identical to any one of SEQ ID NOs: 3, 82 to 116, 142, and 143, or a portion thereof; and/or
the TCR-b variable region comprises a sequence that is at least 70%, 80%, 85%, 90%, 95%, 97%, 98%, 99%, or 100% identical to any one of SEQ ID NOs: 10, 117 to 121, and 144, or a portion thereof; optionally wherein
the TCR-a variable region comprises a sequence that is at least 70%, 80%, 85%, 90%, 95%, 97%, 98%, 99%, or 100% identical to any one of SEQ ID NOs: 3 and 82 to 116, or a portion thereof; and/or
the TCR-b variable region comprises a sequence that is at least 70%, 80%, 85%, 90%, 95%, 97%, 98%, 99%, or 100% identical to any one of SEQ ID NOs: 10 and 117 to 121, or a portion thereof.

18. A TCR, or an antigen-binding portion thereof, capable of binding specifically to a peptide according to SEQ ID NO: 122 presented in the context of an MHC class I molecule, comprising:
a TCR-a variable region that comprises a sequence that is at least 70%, 80%, 85%, 90%, 95%, 97%, 98%, 99%, or 100% identical to any one of SEQ ID NOs: 3, 82 to 116, 142, and 143, or a portion thereof; and
a TCR-b variable region that comprises a sequence that is at least 70%, 80%, 85%, 90%, 95%, 97%, 98%, 99%, or 100% identical to any one of SEQ ID NOs: 10, 117 to 121, and 144, or a portion thereof;
wherein the TCR does not comprise both SEQ ID NO: 3 and SEQ ID NO: 10; optionally wherein
the TCR-a variable region comprises a sequence that is at least 70%, 80%, 85%, 90%, 95%, 97%, 98%, 99%, or 100% identical to any one of SEQ ID NOs: 3 and 82 to 116, or a portion thereof; and
the TCR-b variable region comprises a sequence that is at least 70%, 80%, 85%, 90%, 95%, 97%, 98%, 99%, or 100% identical to any one of SEQ ID NOs: 10 and 117 to 121, or a portion thereof.

19. A TCR, or an antigen-binding portion thereof, capable of binding specifically to a peptide according to SEQ ID NO: 122 presented in the context of an MHC class I molecule, comprising:
a TCR-a variable region that comprises a sequence that is at least 70%, 80%, 85%, 90%, 95%, 97%, 98%, 99%, or 100% identical to any one of SEQ ID NOs: 3, 82 to 116, 142, and 143, or a portion thereof; and
a TCR-b variable region that comprises a sequence that is at least 70%, 80%, 85%, 90%, 95%, 97%, 98%, 99%, or 100% identical to any one of SEQ ID NOs: 10, 117 to 121, and 144, or a portion thereof;
wherein TCR-a variable region sequence and the TCR-b variable region sequence are associated with the same TCR ID listed in Tables 3 and 4.

20. A TCR, or an antigen-binding portion thereof, capable of binding specifically to a peptide according to SEQ ID NO: 122 presented in the context of an MHC class I molecule, comprising:
a TCR-a variable region, or portion thereof, and a TCR-b variable region, or portion thereof, wherein
(1) the TCRa variable region comprises a sequence that is at least 70%, 80%, 85%, 90%, 95%, 97%, 98%, 99%, or 100% identical to SEQ ID NO: 82 and the TCRb variable region comprises a sequence that is at least 70%, 80%, 85%, 90%, 95%, 97%, 98%, 99%, or 100% identical to SEQ ID NO: 10; or
(2) the TCRa variable region comprises a sequence that is at least 70%, 80%, 85%, 90%, 95%, 97%, 98%, 99%, or 100% identical to SEQ ID NO: 83 and the TCRb variable region comprises a sequence that is at least 70%, 80%, 85%, 90%, 95%, 97%, 98%, 99%, or 100% identical to SEQ ID NO: 10; or
(3) the TCRa variable region comprises a sequence that is at least 70%, 80%, 85%, 90%, 95%, 97%, 98%, 99%, or 100% identical to SEQ ID NO: 84 and the TCRb variable region comprises a sequence that is at least 70%, 80%, 85%, 90%, 95%, 97%, 98%, 99%, or 100% identical to SEQ ID NO: 10; or
(4) the TCRa variable region comprises a sequence that is at least 70%, 80%, 85%, 90%, 95%, 97%, 98%, 99%, or 100% identical to SEQ ID NO: 85 and the TCRb variable region comprises a sequence that is at least 70%, 80%, 85%, 90%, 95%, 97%, 98%, 99%, or 100% identical to SEQ ID NO: 10; or
(5) the TCRa variable region comprises a sequence that is at least 70%, 80%, 85%, 90%, 95%, 97%, 98%, 99%, or 100% identical to SEQ ID NO: 86 and the TCRb variable region comprises a sequence that is at least 70%, 80%, 85%, 90%, 95%, 97%, 98%, 99%, or 100% identical to SEQ ID NO: 10; or
(6) the TCRa variable region comprises a sequence that is at least 70%, 80%, 85%, 90%, 95%, 97%, 98%, 99%, or 100% identical to SEQ ID NO: 87 and the TCRb variable region comprises a sequence that is at least 70%, 80%, 85%, 90%, 95%, 97%, 98%, 99%, or 100% identical to SEQ ID NO: 10; or
(7) the TCRa variable region comprises a sequence that is at least 70%, 80%, 85%, 90%, 95%, 97%, 98%, 99%, or 100% identical to SEQ ID NO: 88 and the TCRb variable region comprises a sequence that is at least 70%, 80%, 85%, 90%, 95%, 97%, 98%, 99%, or 100% identical to SEQ ID NO: 117; or
(8) the TCRa variable region comprises a sequence that is at least 70%, 80%, 85%, 90%, 95%, 97%, 98%, 99%, or 100% identical to SEQ ID NO: 89 and the TCRb variable region comprises a sequence that is at least 70%, 80%, 85%, 90%, 95%, 97%, 98%, 99%, or 100% identical to SEQ ID NO: 117; or
(9) the TCRa variable region comprises a sequence that is at least 70%, 80%, 85%, 90%, 95%, 97%, 98%, 99%, or 100% identical to SEQ ID NO: 90 and the TCRb variable region comprises a sequence that is at least 70%, 80%, 85%, 90%, 95%, 97%, 98%, 99%, or 100% identical to SEQ ID NO: 117; or
(10) the TCRa variable region comprises a sequence that is at least 70%, 80%, 85%, 90%, 95%, 97%, 98%, 99%, or 100% identical to SEQ ID NO: 91 and the TCRb variable region comprises a sequence that is at least 70%, 80%, 85%, 90%, 95%, 97%, 98%, 99%, or 100% identical to SEQ ID NO: 117; or
(11) the TCRa variable region comprises a sequence that is at least 70%, 80%, 85%, 90%, 95%, 97%, 98%, 99%, or 100% identical to SEQ ID NO: 92 and the TCRb variable region comprises a sequence that is at least 70%, 80%, 85%, 90%, 95%, 97%, 98%, 99%, or 100% identical to SEQ ID NO: 117; or
(12) the TCRa variable region comprises a sequence that is at least 70%, 80%, 85%, 90%, 95%, 97%, 98%, 99%, or 100% identical to SEQ ID NO: 93 and the TCRb variable region comprises a sequence that is at least 70%, 80%, 85%, 90%, 95%, 97%, 98%, 99%, or 100% identical to SEQ ID NO: 10; or
(13) the TCRa variable region comprises a sequence that is at least 70%, 80%, 85%, 90%, 95%, 97%, 98%, 99%, or 100% identical to SEQ ID NO: 82 and the TCRb variable region comprises a sequence that is at least 70%, 80%, 85%, 90%, 95%, 97%, 98%, 99%, or 100% identical to SEQ ID NO: 118; or
(14) the TCRa variable region comprises a sequence that is at least 70%, 80%, 85%, 90%, 95%, 97%, 98%, 99%, or 100% identical to SEQ ID NO: 83 and the TCRb variable region comprises a sequence that is at least 70%, 80%, 85%, 90%, 95%, 97%, 98%, 99%, or 100% identical to SEQ ID NO: 118; or
(15) the TCRa variable region comprises a sequence that is at least 70%, 80%, 85%, 90%, 95%, 97%, 98%, 99%, or 100% identical to SEQ ID NO: 84 and the TCRb variable region comprises a sequence that is at least 70%, 80%, 85%, 90%, 95%, 97%, 98%, 99%, or 100% identical to SEQ ID NO: 118; or
(16) the TCRa variable region comprises a sequence that is at least 70%, 80%, 85%, 90%, 95%, 97%, 98%, 99%, or 100% identical to SEQ ID NO: 85 and the TCRb variable region comprises a sequence that is at least 70%, 80%, 85%, 90%, 95%, 97%, 98%, 99%, or 100% identical to SEQ ID NO: 118; or
(17) the TCRa variable region comprises a sequence that is at least 70%, 80%, 85%, 90%, 95%, 97%, 98%, 99%, or 100% identical to SEQ ID NO: 86 and the TCRb variable region comprises a sequence that is at least 70%, 80%, 85%, 90%, 95%, 97%, 98%, 99%, or 100% identical to SEQ ID NO: 118; or
(18) the TCRa variable region comprises a sequence that is at least 70%, 80%, 85%, 90%, 95%, 97%, 98%, 99%, or 100% identical to SEQ ID NO: 3 and the TCRb variable region comprises a sequence that is at least 70%, 80%, 85%, 90%, 95%, 97%, 98%, 99%, or 100% identical to SEQ ID NO: 118; or
(19) the TCRa variable region comprises a sequence that is at least 70%, 80%, 85%, 90%, 95%, 97%, 98%, 99%, or 100% identical to SEQ ID NO: 93 and the TCRb variable region comprises a sequence that is at least 70%, 80%, 85%, 90%, 95%, 97%, 98%, 99%, or 100% identical to SEQ ID NO: 118; or
(20) the TCRa variable region comprises a sequence that is at least 70%, 80%, 85%, 90%, 95%, 97%, 98%, 99%, or 100% identical to SEQ ID NO: 94 and the TCRb variable region comprises a sequence that is at least 70%, 80%, 85%, 90%, 95%, 97%, 98%, 99%, or 100% identical to SEQ ID NO: 10; or
(21) the TCRa variable region comprises a sequence that is at least 70%, 80%, 85%, 90%, 95%, 97%, 98%, 99%, or 100% identical to SEQ ID NO: 95 and the TCRb variable region comprises a sequence that is at least 70%, 80%, 85%, 90%, 95%, 97%, 98%, 99%, or 100% identical to SEQ ID NO: 10; or
(22) the TCRa variable region comprises a sequence that is at least 70%, 80%, 85%, 90%, 95%, 97%, 98%, 99%, or 100% identical to SEQ ID NO: 96 and the TCRb variable region comprises a sequence that is at least 70%, 80%, 85%, 90%, 95%, 97%, 98%, 99%, or 100% identical to SEQ ID NO: 10; or
(23) the TCRa variable region comprises a sequence that is at least 70%, 80%, 85%, 90%, 95%, 97%, 98%, 99%, or 100% identical to SEQ ID NO: 97 and the TCRb variable region comprises a sequence that is at least 70%, 80%, 85%, 90%, 95%, 97%, 98%, 99%, or 100% identical to SEQ ID NO: 10; or
(24) the TCRa variable region comprises a sequence that is at least 70%, 80%, 85%, 90%, 95%, 97%, 98%, 99%, or 100% identical to SEQ ID NO: 94 and the TCRb variable region comprises a sequence that is at least 70%, 80%, 85%, 90%, 95%, 97%, 98%, 99%, or 100% identical to SEQ ID NO: 118; or
(25) the TCRa variable region comprises a sequence that is at least 70%, 80%, 85%, 90%, 95%, 97%, 98%, 99%, or 100% identical to SEQ ID NO: 95 and the TCRb variable region comprises a sequence that is at least 70%, 80%, 85%, 90%, 95%, 97%, 98%, 99%, or 100% identical to SEQ ID NO: 118; or
(26) the TCRa variable region comprises a sequence that is at least 70%, 80%, 85%, 90%, 95%, 97%, 98%, 99%, or 100% identical to SEQ ID NO: 96 and the TCRb variable region comprises a sequence that is at least 70%, 80%, 85%, 90%, 95%, 97%, 98%, 99%, or 100% identical to SEQ ID NO: 118; or
(27) the TCRa variable region comprises a sequence that is at least 70%, 80%, 85%, 90%, 95%, 97%, 98%, 99%, or 100% identical to SEQ ID NO: 97 and the TCRb variable region comprises a sequence that is at least 70%, 80%, 85%, 90%, 95%, 97%, 98%, 99%, or 100% identical to SEQ ID NO: 118; or
(28) the TCRa variable region comprises a sequence that is at least 70%, 80%, 85%, 90%, 95%, 97%, 98%, 99%, or 100% identical to SEQ ID NO: 98 and the TCRb variable region comprises a sequence that is at least 70%, 80%, 85%, 90%, 95%, 97%, 98%, 99%, or 100% identical to SEQ ID NO: 118; or
(29) the TCRa variable region comprises a sequence that is at least 70%, 80%, 85%, 90%, 95%, 97%, 98%, 99%, or 100% identical to SEQ ID NO: 99 and the TCRb variable region comprises a sequence that is at least 70%, 80%, 85%, 90%, 95%, 97%, 98%, 99%, or 100% identical to SEQ ID NO: 10; or
(30) the TCRa variable region comprises a sequence that is at least 70%, 80%, 85%, 90%, 95%, 97%, 98%, 99%, or 100% identical to SEQ ID NO: 100 and the TCRb variable region comprises a sequence that is at least 70%, 80%, 85%, 90%, 95%, 97%, 98%, 99%, or 100% identical to SEQ ID NO: 10; or
(31) the TCRa variable region comprises a sequence that is at least 70%, 80%, 85%, 90%, 95%, 97%, 98%, 99%, or 100% identical to SEQ ID NO: 101 and the TCRb variable region comprises a sequence that is at least 70%, 80%, 85%, 90%, 95%, 97%, 98%, 99%, or 100% identical to SEQ ID NO: 10; or
(32) the TCRa variable region comprises a sequence that is at least 70%, 80%, 85%, 90%, 95%, 97%, 98%, 99%, or 100% identical to SEQ ID NO: 102 and the TCRb variable region comprises a sequence that is at least 70%, 80%, 85%, 90%, 95%, 97%, 98%, 99%, or 100% identical to SEQ ID NO: 10; or
(33) the TCRa variable region comprises a sequence that is at least 70%, 80%, 85%, 90%, 95%, 97%, 98%, 99%, or 100% identical to SEQ ID NO: 103 and the TCRb variable region comprises a sequence that is at least 70%, 80%, 85%, 90%, 95%, 97%, 98%, 99%, or 100% identical to SEQ ID NO: 10; or
(34) the TCRa variable region comprises a sequence that is at least 70%, 80%, 85%, 90%, 95%, 97%, 98%, 99%, or 100% identical to SEQ ID NO: 104 and the TCRb variable region comprises a sequence that is at least 70%, 80%, 85%, 90%, 95%, 97%, 98%, 99%, or 100% identical to SEQ ID NO: 10; or
(35) the TCRa variable region comprises a sequence that is at least 70%, 80%, 85%, 90%, 95%, 97%, 98%, 99%, or 100% identical to SEQ ID NO: 105 and the TCRb variable region comprises a sequence that is at least 70%, 80%, 85%, 90%, 95%, 97%, 98%, 99%, or 100% identical to SEQ ID NO: 10; or
(36) the TCRa variable region comprises a sequence that is at least 70%, 80%, 85%, 90%, 95%, 97%, 98%, 99%, or 100% identical to SEQ ID NO: 10⁶ and the TCRb variable region comprises a sequence that is at least 70%, 80%, 85%, 90%, 95%, 97%, 98%, 99%, or 100% identical to SEQ ID NO: 10; or
(37) the TCRa variable region comprises a sequence that is at least 70%, 80%, 85%, 90%, 95%, 97%, 98%, 99%, or 100% identical to SEQ ID NO: 107 and the TCRb variable region comprises a sequence that is at least 70%, 80%, 85%, 90%, 95%, 97%, 98%, 99%, or 100% identical to SEQ ID NO: 119; or
(38) the TCRa variable region comprises a sequence that is at least 70%, 80%, 85%, 90%, 95%, 97%, 98%, 99%, or 100% identical to SEQ ID NO: 108 and the TCRb variable region comprises a sequence that is at least 70%, 80%, 85%, 90%, 95%, 97%, 98%, 99%, or 100% identical to SEQ ID NO: 10; or
(39) the TCRa variable region comprises a sequence that is at least 70%, 80%, 85%, 90%, 95%, 97%, 98%, 99%, or 100% identical to SEQ ID NO: 109 and the TCRb variable region comprises a sequence that is at least 70%, 80%, 85%, 90%, 95%, 97%, 98%, 99%, or 100% identical to SEQ ID NO: 10; or
(40) the TCRa variable region comprises a sequence that is at least 70%, 80%, 85%, 90%, 95%, 97%, 98%, 99%, or 100% identical to SEQ ID NO: 110 and the TCRb variable region comprises a sequence that is at least 70%, 80%, 85%, 90%, 95%, 97%, 98%, 99%, or 100% identical to SEQ ID NO: 10; or
(41) the TCRa variable region comprises a sequence that is at least 70%, 80%, 85%, 90%, 95%, 97%, 98%, 99%, or 100% identical to SEQ ID NO: 111 and the TCRb variable region comprises a sequence that is at least 70%, 80%, 85%, 90%, 95%, 97%, 98%, 99%, or 100% identical to SEQ ID NO: 10; or
(42) the TCRa variable region comprises a sequence that is at least 70%, 80%, 85%, 90%, 95%, 97%, 98%, 99%, or 100% identical to SEQ ID NO: 112 and the TCRb variable region comprises a sequence that is at least 70%, 80%, 85%, 90%, 95%, 97%, 98%, 99%, or 100% identical to SEQ ID NO: 10; or
(43) the TCRa variable region comprises a sequence that is at least 70%, 80%, 85%, 90%, 95%, 97%, 98%, 99%, or 100% identical to SEQ ID NO: 113 and the TCRb variable region comprises a sequence that is at least 70%, 80%, 85%, 90%, 95%, 97%, 98%, 99%, or 100% identical to SEQ ID NO: 10; or
(44) the TCRa variable region comprises a sequence that is at least 70%, 80%, 85%, 90%, 95%, 97%, 98%, 99%, or 100% identical to SEQ ID NO: 114 and the TCRb variable region comprises a sequence that is at least 70%, 80%, 85%, 90%, 95%, 97%, 98%, 99%, or 100% identical to SEQ ID NO: 10; or
(45) the TCRa variable region comprises a sequence that is at least 70%, 80%, 85%, 90%, 95%, 97%, 98%, 99%, or 100% identical to SEQ ID NO: 115 and the TCRb variable region comprises a sequence that is at least 70%, 80%, 85%, 90%, 95%, 97%, 98%, 99%, or 100% identical to SEQ ID NO: 118; or
(46) the TCRa variable region comprises a sequence that is at least 70%, 80%, 85%, 90%, 95%, 97%, 98%, 99%, or 100% identical to SEQ ID NO: 115 and the TCRb variable region comprises a sequence that is at least 70%, 80%, 85%, 90%, 95%, 97%, 98%, 99%, or 100% identical to SEQ ID NO: 10; or
(47) the TCRa variable region comprises a sequence that is at least 70%, 80%, 85%, 90%, 95%, 97%, 98%, 99%, or 100% identical to SEQ ID NO: 114 and the TCRb variable region comprises a sequence that is at least 70%, 80%, 85%, 90%, 95%, 97%, 98%, 99%, or 100% identical to SEQ ID NO: 10; or
(48) the TCRa variable region comprises a sequence that is at least 70%, 80%, 85%, 90%, 95%, 97%, 98%, 99%, or 100% identical to SEQ ID NO: 116 and the TCRb variable region comprises a sequence that is at least 70%, 80%, 85%, 90%, 95%, 97%, 98%, 99%, or 100% identical to SEQ ID NO: 10; or
(49) the TCRa variable region comprises a sequence that is at least 70%, 80%, 85%, 90%, 95%, 97%, 98%, 99%, or 100% identical to SEQ ID NO: 116 and the TCRb variable region comprises a sequence that is at least 70%, 80%, 85%, 90%, 95%, 97%, 98%, 99%, or 100% identical to SEQ ID NO: 120; or
(50) the TCRa variable region comprises a sequence that is at least 70%, 80%, 85%, 90%, 95%, 97%, 98%, 99%, or 100% identical to SEQ ID NO: 93 and the TCRb variable region comprises a sequence that is at least 70%, 80%, 85%, 90%, 95%, 97%, 98%, 99%, or 100% identical to SEQ ID NO: 121; or
(51) the TCRa variable region comprises a sequence that is at least 70%, 80%, 85%, 90%, 95%, 97%, 98%, 99%, or 100% identical to SEQ ID NO: 142 and the TCRb variable region comprises a sequence that is at least 70%, 80%, 85%, 90%, 95%, 97%, 98%, 99%, or 100% identical to SEQ ID NO: 10; or
(52) the TCRa variable region comprises a sequence that is at least 70%, 80%, 85%, 90%, 95%, 97%, 98%, 99%, or 100% identical to SEQ ID NO: 143 and the TCRb variable region comprises a sequence that is at least 70%, 80%, 85%, 90%, 95%, 97%, 98%, 99%, or 100% identical to SEQ ID NO: 10; or
(53) the TCRa variable region comprises a sequence that is at least 70%, 80%, 85%, 90%, 95%, 97%, 98%, 99%, or 100% identical to SEQ ID NO: 142 and the TCRb variable region comprises a sequence that is at least 70%, 80%, 85%, 90%, 95%, 97%, 98%, 99%, or 100% identical to SEQ ID NO: 119; or
(54) the TCRa variable region comprises a sequence that is at least 70%, 80%, 85%, 90%, 95%, 97%, 98%, 99%, or 100% identical to SEQ ID NO: 98 and the TCRb variable region comprises a sequence that is at least 70%, 80%, 85%, 90%, 95%, 97%, 98%, 99%, or 100% identical to SEQ ID NO: 144.

21. The TCR, or antigen-binding portion thereof, of any one of claims 18 to 20, wherein:
(i) the CDR3a sequence is invariant and the CDR3b sequence is invariant, wherein the CDR3a sequence is defined in Table 3 and the CDR3b sequence is defined in Table 4; and/or
(ii) the CDR1a, CDR2a, and CDR3a sequences are invariant and the CDR1b, CDR2b, and CDR3b sequences are invariant; wherein the CDR sequences are defined in Table 3 and Table 4; and/or
(iii) the relevant mutations recited in Table 6 are retained.

22. The TCR, or antigen-binding portion thereof, of any one of claims 1 to 21, wherein the MHC class I molecule is HLA-A*0201.

23. The TCR, or antigen-binding portion thereof, of any one of claims 1 to 22, wherein the TCR, or antigen-binding portion thereof, has a Kd less than or equal to 200 μM, 175 μM, 143 μM, 100 μM, 75 μM, 50 μM, 25 μM, 20 μM, 15 μM, 10 UM, 5 μM, or 2 μM.

24. The TCR, or antigen-binding portion thereof, of any one of claims 1 to 23, wherein the TCR, or antigen-binding portion thereof, comprises a TCRa constant region and/or a TCRb constant region.

25. The TCR, or antigen-binding portion thereof, of claim 24, wherein
the TCRa constant region sequence is 70%, 80%, 85%, 90%, 95%, 97%, 98%, 99%, or 100% identical to SEQ ID NO: 4; and/or
the TCRb constant region sequence is 70%, 80%, 85%, 90%, 95%, 97%, 98%, 99%, or 100% identical to any one of SEQ ID NOs: 11, 138, or 139.

26. The TCR, or antigen-binding portion thereof, of any one of claims 1 to 25, wherein the TCR, or antigen-binding portion thereof, is linked to an agent.

27. The TCR, or antigen-binding portion thereof, of claim 26, wherein the agent is a therapeutic agent.

28. A nucleic acid molecule encoding:
(a) the TCRa variable region amino acid sequence;
(b) the TCRb variable region amino acid sequence; or
(c) both the TCRa variable region and the TCRb variable region amino acid sequences of the TCR or antigen-binding portion of any one of claims 1 to 25.

29. A vector comprising a nucleic acid of claim 28.

30. A cell comprising the TCR, or antigen-binding portion thereof, of any one of claims 1 to 27, the nucleic acid of claim 28, or the vector of claim 29.

31. The cell of claim 30, wherein the cell is an immune cell.

32. The cell of claim 31, wherein the immune cell is a T cell or an NK cell.

33. A pharmaceutical composition comprising the TCR, or antigen-binding portion thereof, of any one of claims 1 to 27, the nucleic acid of claim 28, the vector of claim 29, or the cell of any one of claims 30 to 32.

34. A TCR, or antigen-binding portion thereof, of any one of claims 1 to 27, a nucleic acid of claim 28, a vector of claim 29, a cell of any one of claims 30 to 32, or a pharmaceutical composition of claim 33, for use as a medicament.

35. A TCR, or antigen-binding portion thereof, of any one of claims 1 to 27, a nucleic acid of claim 28, a vector of claim 29, a cell of any one of claims 30 to 32, or a pharmaceutical composition of claim 33, for use in a method for treating, preventing, or ameliorating cancer.

36. The TCR, antigen-binding portion thereof, nucleic acid, vector, cell, or pharmaceutical composition for use according to claim 35, wherein the cancer comprises cells that express MAGE-A4.

37. The TCR, antigen-binding portion thereof, nucleic acid, vector, cell, or pharmaceutical composition for use according to claim 35 or claim 36, wherein the cancer is non-small cell lung cancer (NSCLC), ovarian, gastroesophageal, head and neck synovial sarcoma, or urothelial.
